SEQUENCE LISTING (233 sequences)

SEQ ID NO: 1 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - DNA, 1225 bp, Homo sapiens
  source 1..1225: mol_type = unassigned DNA
atgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtc

SEQ ID NO: 3 - protein, 406 aa, Macaca fascicularis
MRGAPTAGAALMLCVATAVLLRAQGGPVQSKSPRFASWDEMNVLAHGLLQLGQGLREHAERTRSQLNALERRLSACGSACQGTEGSTALPLAPESRVDPEVLHSLQTQLKAQNSRIQQLFHKVAQQQRHLEKQHLRIQRLQSQVGLLDPKHLDHEVAKPARRKRRPEMAQPVDSAHNASRLHRLPRDCQELFEDGERQSGLFEIQPQGSPPFLVNCKMTSDGGWTVIQRRHDGSVDFNRPWEAYKAGFGDPQGEFWLGLEKVHSITGDRNSRLAVQLQDWDGNAESLQFSVHLGGEDTAYSLQLTEPVASQLGATTVPPSGLSVPFSTWDQDHDLRRDKNCAKSLSGGWWFGTCSHSNLNGQYFRSIPQQRQELKKGIFWKTWRGRYYPLQATTMLIQPTAAEAAS

SEQ ID NO: 4 - DNA, 1221 bp, Macaca fascicularis
  source 1..1221: mol_type = unassigned DNA
atgcgcggtgctccgacggccggagcagccctgatgctctgcgtcgccacggccgtgctgctgagagctcagggcggcccggtgcagtccaagtctccgcgctttgcgtcctgggacgagatgaatgtcctggcgcacggactcctgcagctaggccaggggctgcgcgaacacgcggagcgcacccgcagtcagctgaacgcgctggagcggcgcctcagcgcttgcgggtctgcctgccagggaaccgaggggtccaccgccctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcgtctgcaaagccaggttggcctcctggaccccaagcacctagaccatgaggtggccaagcctgcccgaagaaagaggcggcccgagatggcccagccagttgactcggctcacaatgccagccgcctgcaccggctgcccagggattgccaggagctgtttgaagatggggagaggcagagtggactatttgagatccagcctcaggggtctccgccatttttggtgaactgcaagatgacctcagatggaggctggacagtaattcagaggcgccacgatggctctgtggacttcaaccggccctgggaagcctacaaggcggggtttggggatccccaaggcgagttctggctgggcctggagaaggtgcatagcatcacaggggaccgcaacagccgcctggccgtgcagctgcaggactgggatggcaacgccgagtcgctgcagttctctgtgcacctgggtggcgaggacacggcttacagcctgcagctcaccgagcccgtggccagccagttgggtgccaccaccgtcccgcctagcggcctctccgtacccttctccacttgggaccaggatcacgacctccgcagggacaagaactgcgccaagagcctctctggaggctggtggtttggcacctgcagccattccaacctcaatggccagtacttccgctccatcccacagcagcggcaggagcttaagaaaggaatcttctggaagacctggcggggccgctactacccgctgcaggccaccaccatgttgatccagcccacggcggcagaggcagcctcctag

SEQ ID NO: 5 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 6 - DNA, 1380 bp, Homo sapiens
  source 1..1380: mol_type = unassigned DNA
atgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaa

SEQ ID NO: 7 - protein, 5 aa, synthetic construct
  REGION 1..5: note = Description of Artificial Sequence: Synthetic peptide
SSWMQ

SEQ ID NO: 8 - protein, 17 aa, synthetic construct
  REGION 1..17: note = Description of Artificial Sequence: Synthetic peptide
EIDPSDNYANYNQKFQG

SEQ ID NO: 9 - protein, 10 aa, synthetic construct
  REGION 1..10: note = Description of Artificial Sequence: Synthetic peptide
GSYFSNFFDY

SEQ ID NO: 10 - protein, 7 aa, synthetic construct
  REGION 1..7: note = Description of Artificial Sequence: Synthetic peptide
AYTFTSS

SEQ ID NO: 11 - protein, 6 aa, synthetic construct
  REGION 1..6: note = Description of Artificial Sequence: Synthetic peptide
DPSDNY

SEQ ID NO: 12 - protein, 10 aa, synthetic construct
  REGION 1..10: note = Description of Artificial Sequence: Synthetic peptide
GSYFSNFFDY

SEQ ID NO: 13 - protein, 119 aa, synthetic construct
  REGION 1..119: note = Description of Artificial Sequence: Synthetic polypeptide
QVQLVQSGAEVKKPGASVKVSCKASAYTFTSSWMQWVRQAPGQGLEWMGEIDPSDNYANYNQKFQGRVTLTVDTSTSTAYMELSSLRSEDTAVYYCASGSYFSNFFDYWGQGTLVTVSS

SEQ ID NO: 14 - DNA, 357 bp, synthetic construct
  misc_feature 1..357: note = Description of Artificial Sequence: Synthetic polynucleotide
  source 1..357: mol_type = other DNA
caggtgcagctggtgcagtctggcgccgaagtgaagaaaccaggcgccagcgtgaaggtgtcctgcaaggccagcgcctacacctttaccagcagctggatgcagtgggtgcgccaggctcctggacagggcctggaatggatgggcgagatcgaccccagcgacaactacgccaactacaaccagaaattccagggcagagtgaccctgaccgtggacaccagcacctccaccgcctacatggaactgagcagcctgcggagcgaggacaccgccgtgtactattgtgccagcggcagctacttcagcaacttcttcgactactggggccagggcaccctcgtgaccgtgtcatct

SEQ ID NO: 15 - protein, 449 aa, synthetic construct
  REGION 1..449: note = Description of Artificial Sequence: Synthetic polypeptide
QVQLVQSGAEVKKPGASVKVSCKASAYTFTSSWMQWVRQAPGQGLEWMGEIDPSDNYANYNQKFQGRVTLTVDTSTSTAYMELSSLRSEDTAVYYCASGSYFSNFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1347 bp, synthetic construct
  misc_feature 1..1347: note = Description of Artificial Sequence: Synthetic polynucleotide
  source 1..1347: mol_type = other DNA
caggtgcagctggtgcagtctggcgccgaagtgaagaaaccaggcgccagcgtgaaggtgtcctgcaaggccagcgcctacacctttaccagcagctggatgcagtgggtgcgccaggctcctggacagggcctggaatggatgggcgagatcgaccccagcgacaactacgccaactacaaccagaaattccagggcagagtgaccctgaccgtggacaccagcacctccaccgcctacatggaactgagcagcctgcggagcgaggacaccgccgtgtactattgtgccagcggcagctacttcagcaacttcttcgactactggggccagggcaccctcgtgaccgtgtcatctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 17 - protein, 11 aa, synthetic construct
  REGION 1..11: note = Description of Artificial Sequence: Synthetic peptide
KASQDIGSNLN

SEQ ID NO: 18 - protein, 7 aa, synthetic construct
  REGION 1..7: note = Description of Artificial Sequence: Synthetic peptide
AVSNRGP

SEQ ID NO: 19 - protein, 9 aa, synthetic construct
  REGION 1..9: note = Description of Artificial Sequence: Synthetic peptide
LQYASSPWT

SEQ ID NO: 20 - protein, 7 aa, synthetic construct
  REGION 1..7: note = Description of Artificial Sequence: Synthetic peptide
SQDIGSN

SEQ ID NO: 21 - skipped

SEQ ID NO: 22 - protein, 6 aa, synthetic construct
  REGION 1..6: note = Description of Artificial Sequence: Synthetic peptide
YASSPW

SEQ ID NO: 23 - protein, 107 aa, synthetic construct
  REGION 1..107: note = Description of Artificial Sequence: Synthetic polypeptide
EIVMTQSPATLSVSPGERATLSCKASQDIGSNLNWLQQKPGQAPRRLIYAVSNRGPGIPARFSGSRSGSEYTLTISSLQSEDFAVYYCLQYASSPWTFGQGTKVEIK

SEQ ID NO: 24 - DNA, 321 bp, synthetic construct
  misc_feature 1..321: note = Description of Artificial Sequence: Synthetic polynucleotide
  source 1..321: mol_type = other DNA
gagatcgtgatgacacagagccccgccaccctgtccgtgtctccaggcgaaagagccaccctgagctgcaaagccagccaggacatcggcagcaacctgaactggctgcagcagaaaccaggccaggcccccagaaggctgatctacgctgtttccaaccgtggtcctggcatccccgccagattttccggcagcagatccggcagcgagtacaccctgaccatcagcagcctgcagagcgaggacttcgccgtgtactactgcctgcagtacgccagcagcccctggacatttggccagggcaccaaggtggaaatcaag

SEQ ID NO: 25 - protein, 214 aa, synthetic construct
  REGION 1..214: note = Description of Artificial Sequence: Synthetic polypeptide
EIVMTQSPATLSVSPGERATLSCKASQDIGSNLNWLQQKPGQAPRRLIYAVSNRGPGIPARFSGSRSGSEYTLTISSLQSEDFAVYYCLQYASSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 642 bp, synthetic construct
  misc_feature 1..642: note = Description of Artificial Sequence: Synthetic polynucleotide
  source 1..642: mol_type = other DNA
gagatcgtgatgacacagagccccgccaccctgtccgtgtctccaggcgaaagagccaccctgagctgcaaagccagccaggacatcggcagcaacctgaactggctgcagcagaaaccaggccaggcccccagaaggctgatctacgctgtttccaaccgtggtcctggcatccccgccagattttccggcagcagatccggcagcgagtacaccctgaccatcagcagcctgcagagcgaggacttcgccgtgtactactgcctgcagtacgccagcagcccctggacatttggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 27 - DNA, 357 bp, synthetic construct
  misc_feature 1..357: note = Description of Artificial Sequence: Synthetic polynucleotide
  source 1..357: mol_type = other DNA
caggtgcagctggtgcagtcaggcgccgaagtgaagaaacccggcgctagtgtgaaagtcagctgtaaagctagtgcctacaccttcacctctagctggatgcagtgggtcagacaggccccaggtcagggcctggagtggatgggcgagatcgaccctagcgataactacgctaactataatcagaagtttcagggtagagtcaccctgaccgtggacactagcactagcaccgcctatatggaactgtctagcctgagatcagaggacaccgccgtctactactgcgctagtggtagctacttctctaacttcttcgactactggggtcagggcaccctggtcaccgtgtctagc

SEQ ID NO: 28 - protein, 449 aa, synthetic construct
  REGION 1..449: note = Description of Artificial Sequence: Synthetic polypeptide
QVQLVQSGAEVKKPGASVKVSCKASAYTFTSSWMQWVRQAPGQGLEWMGEIDPSDNYANYNQKFQGRVTLTVDTSTSTAYMELSSLRSEDTAVYYCASGSYFSNFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1347 bp, synthetic construct
  misc_feature 1..1347: note = Description of Artificial Sequence: Synthetic polynucleotide
  source 1..1347: mol_type = other DNA
caggtgcagctggtgcagtcaggcgccgaagtgaagaaacccggcgctagtgtgaaagtcagctgtaaagctagtgcctacaccttcacctctagctggatgcagtgggtcagacaggccccaggtcagggcctggagtggatgggcgagatcgaccctagcgataactacgctaactataatcagaagtttcagggtagagtcaccctgaccgtggacactagcactagcaccgcctatatggaactgtctagcctgagatcagaggacaccgccgtctactactgcgctagtggtagctacttctctaacttcttcgactactggggtcagggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 30 - DNA, 321 bp, synthetic construct
  misc_feature 1..321: note = Description of Artificial Sequence: Synthetic polynucleotide
  source 1..321: mol_type = other DNA
gagatcgtgatgactcagtcacccgctaccctgagcgtcagccctggcgagcgggctacactgagctgtaaagcctctcaggatatcggctctaacctgaactggctgcagcagaagcccggtcaggcccctagacggctgatctacgccgtgtctaatagaggccccggaatccccgctaggtttagcggctctaggtcaggttcagagtacaccctgactatctctagcctgcagtcagaggacttcgccgtctactactgcctgcagtacgcctctagcccctggaccttcggtcagggcactaaggtcgagattaag

SEQ ID NO: 31 - DNA, 642 bp, synthetic construct
  misc_feature 1..642: note = Description of Artificial Sequence: Synthetic polynucleotide
  source 1..642: mol_type = other DNA
gagatcgtgatgactcagtcacccgctaccctgagcgtcagccctggcgagcgggctacactgagctgtaaagcctctcaggatatcggctctaacctgaactggctgcagcagaagcccggtcaggcccctagacggctgatctacgccgtgtctaatagaggccccggaatccccgctaggtttagcggctctaggtcaggttcagagtacaccctgactatctctagcctgcagtcagaggacttcgccgtctactactgcctgcagtacgcctctagcccctggaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 32 - protein, 5 aa, synthetic construct
  REGION 1..5: note = Description of Artificial Sequence: Synthetic peptide
SSWMQ

SEQ ID NO: 33 - protein, 17 aa, synthetic construct
  REGION 1..17: note = Description of Artificial Sequence: Synthetic peptide
EIDPSDNYANYNQKFQG

SEQ ID NO: 34 - protein, 10 aa, synthetic construct
  REGION 1..10: note = Description of Artificial Sequence: Synthetic peptide
GSYFSNFFDY

SEQ ID NO: 35 - protein, 7 aa, synthetic construct
  REGION 1..7: note = Description of Artificial Sequence: Synthetic peptide
AYTFTSS

SEQ ID NO: 36 - protein, 6 aa, synthetic construct
  REGION 1..6: note = Description of Artificial Sequence: Synthetic peptide
DPSDNY

SEQ ID NO: 37 - protein, 10 aa, synthetic construct
  REGION 1..10: note = Description of Artificial Sequence: Synthetic peptide
GSYFSNFFDY

SEQ ID NO: 38 - protein, 119 aa, synthetic construct; identical to SEQ ID NO: 13
  REGION 1..119: note = Description of Artificial Sequence: Synthetic polypeptide

SEQ ID NO: 39 - DNA, 357 bp, synthetic construct; identical to SEQ ID NO: 14
  misc_feature 1..357: note = Description of Artificial Sequence: Synthetic polynucleotide
  source 1..357: mol_type = other DNA

SEQ ID NO: 40 - protein, 449 aa, synthetic construct; identical to SEQ ID NO: 15
  REGION 1..449: note = Description of Artificial Sequence: Synthetic polypeptide

SEQ ID NO: 41 - DNA, 1347 bp, synthetic construct; identical to SEQ ID NO: 16
  misc_feature 1..1347: note = Description of Artificial Sequence: Synthetic polynucleotide
  source 1..1347: mol_type = other DNA

SEQ ID NO: 42 - protein, 11 aa, synthetic construct
  REGION 1..11: note = Description of Artificial Sequence: Synthetic peptide
KASQDIGSNLN

SEQ ID NO: 43 - protein, 7 aa, synthetic construct
  REGION 1..7: note = Description of Artificial Sequence: Synthetic peptide
AASVREP

SEQ ID NO: 44 - protein, 9 aa, synthetic construct
  REGION 1..9: note = Description of Artificial Sequence: Synthetic peptide
LQYASSPWT

SEQ ID NO: 45 - protein, 7 aa, synthetic construct
  REGION 1..7: note = Description of Artificial Sequence: Synthetic peptide
SQDIGSN

SEQ ID NO: 46 - skipped

SEQ ID NO: 47 - protein, 6 aa, synthetic construct
  REGION 1..6: note = Description of Artificial Sequence: Synthetic peptide
YASSPW

SEQ ID NO: 48 - protein, 107 aa, synthetic construct
  REGION 1..107: note = Description of Artificial Sequence: Synthetic polypeptide
EIVMTQSPATLSVSPGERATLSCKASQDIGSNLNWLQQKPGQAPRRLIYAASVREPGIPARFSGSRSGSEYTLTISSLQSEDFAVYYCLQYASSPWTFGQGTKVEIK

SEQ ID NO: 49 - DNA, 321 bp, synthetic construct
  misc_feature 1..321: note = Description of Artificial Sequence: Synthetic polynucleotide
  source 1..321: mol_type = other DNA
gagatcgtgatgacacagagccccgccaccctgtccgtgtctccaggcgaaagagccaccctgagctgcaaagccagccaggacatcggcagcaacctgaactggctgcagcagaaaccaggccaggcccccagaaggctgatctacgctgcttccgtccgtgagcctggcatccccgccagattttccggcagcagatccggcagcgagtacaccctgaccatcagcagcctgcagagcgaggacttcgccgtgtactactgcctgcagtacgccagcagcccctggacatttggccagggcaccaaggtggaaatcaag

SEQ ID NO: 50 - protein, 214 aa, synthetic construct
  REGION 1..214: note = Description of Artificial Sequence: Synthetic polypeptide
EIVMTQSPATLSVSPGERATLSCKASQDIGSNLNWLQQKPGQAPRRLIYAASVREPGIPARFSGSRSGSEYTLTISSLQSEDFAVYYCLQYASSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - DNA, 642 bp, synthetic construct
  misc_feature 1..642: note = Description of Artificial Sequence: Synthetic polynucleotide
  source 1..642: mol_type = other DNA
gagatcgtgatgacacagagccccgccaccctgtccgtgtctccaggcgaaagagccaccctgagctgcaaagccagccaggacatcggcagcaacctgaactggctgcagcagaaaccaggccaggcccccagaaggctgatctacgctgcttccgtccgtgagcctggcatccccgccagattttccggcagcagatccggcagcgagtacaccctgaccatcagcagcctgcagagcgaggacttcgccgtgtactactgcctgcagtacgccagcagcccctggacatttggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 52 - protein, 5 aa, synthetic construct
  REGION 1..5: note = Description of Artificial Sequence: Synthetic peptide
SYTMH

SEQ ID NO: 53 - protein, 17 aa, synthetic construct
  REGION 1..17: note = Description of Artificial Sequence: Synthetic peptide
YINPSSGYTKYNQKFQG

SEQ ID NO: 54 - protein, 9 aa, synthetic construct
  REGION 1..9: note = Description of Artificial Sequence: Synthetic peptide
GWLLLAMDY

SEQ ID NO: 55 - protein, 7 aa, synthetic construct
  REGION 1..7: note = Description of Artificial Sequence: Synthetic peptide
GYTFTSY

SEQ ID NO: 56 - protein, 6 aa, synthetic construct
  REGION 1..6: note = Description of Artificial Sequence: Synthetic peptide
NPSSGY

SEQ ID NO: 57 - protein, 9 aa, synthetic construct
  REGION 1..9: note = Description of Artificial Sequence: Synthetic peptide
GWLLLAMDY

SEQ ID NO: 58 - protein, 118 aa, synthetic construct
  REGION 1..118: note = Description of Artificial Sequence: Synthetic polypeptide
QVQLVQSGAEVKKPGASVKVSCKASGYTFTSYTMHWVRQAPGQGLEWMGYINPSSGYTKYNQKFQGRVTMTADKSTSTAYMELSSLRSEDTAVYYCAEGWLLLAMDYWGQGTLVTVSS

SEQ ID NO: 59 - DNA, 354 bp, synthetic construct
  misc_feature 1..354: note = Description of Artificial Sequence: Synthetic polynucleotide
  source 1..354: mol_type = other DNA
caggtgcagctggtgcagtctggcgccgaagtgaagaaaccaggcgccagcgtgaaggtgtcctgcaaggccagcggctacacctttaccagctacaccatgcactgggtgcgccaggctccaggccagggactggaatggatgggctacatcaaccccagcagcggctataccaagtacaaccagaaattccagggccgcgtgaccatgaccgccgacaagagcacaagcaccgcctacatggaactgagcagcctgcggagcgaggacaccgccgtgtactattgtgccgagggctggctgctgctggccatggattattggggccagggcaccctcgtgaccgtgtctagt

SEQ ID NO: 60 - protein, 448 aa, synthetic construct
  REGION 1..448: note = Description of Artificial Sequence: Synthetic polypeptide
QVQLVQSGAEVKKPGASVKVSCKASGYTFTSYTMHWVRQAPGQGLEWMGYINPSSGYTKYNQKFQGRVTMTADKSTSTAYMELSSLRSEDTAVYYCAEGWLLLAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1344 bp, synthetic construct
  misc_feature 1..1344: note = Description of Artificial Sequence: Synthetic polynucleotide
  source 1..1344: mol_type = other DNA
caggtgcagctggtgcagtctggcgccgaagtgaagaaaccaggcgccagcgtgaaggtgtcctgcaaggccagcggctacacctttaccagctacaccatgcactgggtgcgccaggctccaggccagggactggaatggatgggctacatcaaccccagcagcggctataccaagtacaaccagaaattccagggccgcgtgaccatgaccgccgacaagagcacaagcaccgcctacatggaactgagcagcctgcggagcgaggacaccgccgtgtactattgtgccgagggctggctgctgctggccatggattattggggccagggcaccctcgtgaccgtgtctagtgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 62 - protein, 14 aa, synthetic construct
  REGION 1..14: note = Description of Artificial Sequence: Synthetic peptide
RSSTGAVTTSNYAI

SEQ ID NO: 63 - protein, 7 aa, synthetic construct
  REGION 1..7: note = Description of Artificial Sequence: Synthetic peptide
GTNNRAP

SEQ ID NO: 64 - protein, 9 aa, synthetic construct
  REGION 1..9: note = Description of Artificial Sequence: Synthetic peptide
ALWYSDHWV

SEQ ID NO: 65 - protein, 10 aa, synthetic construct
  REGION 1..10: note = Description of Artificial Sequence: Synthetic peptide
STGAVTTSNY

SEQ ID NO: 66 - skipped

SEQ ID NO: 67 - protein, 6 aa, synthetic construct
  REGION 1..6: note = Description of Artificial Sequence: Synthetic peptide
WYSDHW

SEQ ID NO: 68 - protein, 110 aa, synthetic construct
  REGION 1..110: note = Description of Artificial Sequence: Synthetic polypeptide
EAVVTQSPATLSLSPGERATLSCRSSTGAVTTSNYAIWVQEKPGQAPRGLIGGTNNRAPGIPARFSGSLSGDDATLTISSLQPEDFAVYFCALWYSDHWVFGQGTKVEIK

SEQ ID NO: 69 - DNA, 330 bp, synthetic construct
  misc_feature 1..330: note = Description of Artificial Sequence: Synthetic polynucleotide
  source 1..330: mol_type = other DNA
gaagccgtcgtgacacagagccctgccaccctgtcactgagccctggcgaaagagccaccctgagctgcagatctagcaccggcgctgtgaccaccagcaactacgccatctgggtgcaggaaaagcccggccaggctcccagaggactgatcggcggcaccaacaatagagcccctggcatccccgccagattcagcggatctctgtctggcgacgacgccacactgaccatcagcagcctgcagcccgaggacttcgccgtgtacttctgcgccctgtggtacagcgaccactgggtgttcggccagggcaccaaggtggaaatcaag

SEQ ID NO: 70 - protein, 217 aa, synthetic construct
  REGION 1..217: note = Description of Artificial Sequence: Synthetic polypeptide
EAVVTQSPATLSLSPGERATLSCRSSTGAVTTSNYAIWVQEKPGQAPRGLIGGTNNRAPGIPARFSGSLSGDDATLTISSLQPEDFAVYFCALWYSDHWVFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 651 bp, synthetic construct
  misc_feature 1..651: note = Description of Artificial Sequence: Synthetic polynucleotide
  source 1..651: mol_type = other DNA
gaagccgtcgtgacacagagccctgccaccctgtcactgagccctggcgaaagagccaccctgagctgcagatctagcaccggcgctgtgaccaccagcaactacgccatctgggtgcaggaaaagcccggccaggctcccagaggactgatcggcggcaccaacaatagagcccctggcatccccgccagattcagcggatctctgtctggcgacgacgccacactgaccatcagcagcctgcagcccgaggacttcgccgtgtacttctgcgccctgtggtacagcgaccactgggtgttcggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 72 - protein, 5 aa, synthetic construct
  REGION 1..5: note = Description of Artificial Sequence: Synthetic peptide
NYWIT

SEQ ID NO: 73 - protein, 17 aa, synthetic construct
  REGION 1..17: note = Description of Artificial Sequence: Synthetic peptide
DFYPGGGSTNYNAKLQG

SEQ ID NO: 74 - protein, 10 aa, synthetic construct
  REGION 1..10: note = Description of Artificial Sequence: Synthetic peptide
SPPQVAPFDY

SEQ ID NO: 75 - protein, 7 aa, synthetic construct
  REGION 1..7: note = Description of Artificial Sequence: Synthetic peptide
GYTFNNY

SEQ ID NO: 76 - protein, 6 aa, synthetic construct
  REGION 1..6: note = Description of Artificial Sequence: Synthetic peptide
YPGGGS

SEQ ID NO: 77 - protein, 10 aa, synthetic construct
  REGION 1..10: note = Description of Artificial Sequence: Synthetic peptide
SPPQVAPFDY

SEQ ID NO: 78 - protein, 119 aa, synthetic construct
  REGION 1..119: note = Description of Artificial Sequence: Synthetic polypeptide
QVQLVQSGAEVKKPGASVKVSCKASGYTFNNYWITWVRQAPGQGLEWMGDFYPGGGSTNYNAKLQGRVTLTVDTSTSTAYMELRSLRSDDTAVYYCARSPPQVAPFDYWGQGTLVTVSS

SEQ ID NO: 79 - DNA, 357 bp, synthetic construct
  misc_feature 1..357: note = Description of Artificial Sequence: Synthetic polynucleotide
  source 1..357: mol_type = other DNA
caggtgcagctggtgcagtctggcgccgaagtgaagaaaccaggcgccagcgtgaaggtgtcctgcaaggccagcggctacacctttaacaactactggatcacctgggtgcgccaggcccctggacagggactggaatggatgggcgacttctaccctggcggcggcagcaccaactacaacgccaagctgcagggcagagtgaccctgaccgtggacaccagcacctccaccgcctacatggaactgcggagcctgagaagcgacgacaccgccgtgtattactgcgctagaagccctcctcaggtggcccccttcgattattggggccagggcacactcgtgaccgtgtcctct

SEQ ID NO: 80 - protein, 449 aa, synthetic construct
  REGION 1..449: note = Description of Artificial Sequence: Synthetic polypeptide
QVQLVQSGAEVKKPGASVKVSCKASGYTFNNYWITWVRQAPGQGLEWMGDFYPGGGSTNYNAKLQGRVTLTVDTSTSTAYMELRSLRSDDTAVYYCARSPPQVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1347 bp, synthetic construct
  misc_feature 1..1347: note = Description of Artificial Sequence: Synthetic polynucleotide
  source 1..1347: mol_type = other DNA
caggtgcagctggtgcagtctggcgccgaagtgaagaaaccaggcgccagcgtgaaggtgtcctgcaaggccagcggctacacctttaacaactactggatcacctgggtgcgccaggcccctggacagggactggaatggatgggcgacttctaccctggcggcggcagcaccaactacaacgccaagctgcagggcagagtgaccctgaccgtggacaccagcacctccaccgcctacatggaactgcggagcctgagaagcgacgacaccgccgtgtattactgcgctagaagccctcctcaggtggcccccttcgattattggggccagggcacactcgtgaccgtgtcctctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 82 - protein, 11 aa, synthetic construct
  REGION 1..11: note = Description of Artificial Sequence: Synthetic peptide
QASDYIYHWLG

SEQ ID NO: 83 - protein, 7 aa, synthetic construct
  REGION 1..7: note = Description of Artificial Sequence: Synthetic peptide
GASGLET

SEQ ID NO: 84 - protein, 9 aa, synthetic construct
  REGION 1..9: note = Description of Artificial Sequence: Synthetic peptide
QQYWSTPWT

SEQ ID NO: 85 - protein, 7 aa, synthetic construct
  REGION 1..7: note = Description of Artificial Sequence: Synthetic peptide
SDYIYHW

SEQ ID NO: 86 - skipped

SEQ ID NO: 87 - protein, 6 aa, synthetic construct
  REGION 1..6: note = Description of Artificial Sequence: Synthetic peptide
YWSTPW

SEQ ID NO: 88 - protein, 107 aa, synthetic construct
  REGION 1..107: note = Description of Artificial Sequence: Synthetic polypeptide
DIQMTQSPSSLSASVGDRVTITCQASDYIYHWLGWYQQKPGKAPKLLISGASGLETGVPSRFSGSGSGKDYTFTISSLQPEDIATYYCQQYWSTPWTFGQGTKLEIK

SEQ ID NO: 89 - DNA, 321 bp, synthetic construct
  misc_feature 1..321: note = Description of Artificial Sequence: Synthetic polynucleotide
  source 1..321: mol_type = other DNA
gacatccagatgacccagagccccagcagcctgtctgccagcgtgggcgacagggtgaccatcacctgtcaggccagcgactacatctaccactggctgggctggtatcagcagaagcccggcaaggcccccaagctgctgattagcggagcctccggtctggaaaccggcgtgccaagcagattttccggcagcggctccggcaaggactacaccttcaccatcagctccctgcagcccgaggatatcgccacctactactgccagcagtactggtccaccccctggacctttggccagggcaccaagctggaaatcaag

SEQ ID NO: 90 - protein, 214 aa, synthetic construct
  REGION 1..214: note = Description of Artificial Sequence: Synthetic polypeptide
DIQMTQSPSSLSASVGDRVTITCQASDYIYHWLGWYQQKPGKAPKLLISGASGLETGVPSRFSGSGSGKDYTFTISSLQPEDIATYYCQQYWSTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 642 bp, synthetic construct
  misc_feature 1..642: note = Description of Artificial Sequence: Synthetic polynucleotide
  source 1..642: mol_type = other DNA
gacatccagatgacccagagccccagcagcctgtctgccagcgtgggcgacagggtgaccatcacctgtcaggccagcgactacatctaccactggctgggctggtatcagcagaagcccggcaaggcccccaagctgctgattagcggagcctccggtctggaaaccggcgtgccaagcagattttccggcagcggctccggcaaggactacaccttcaccatcagctccctgcagcccgaggatatcgccacctactactgccagcagtactggtccaccccctggacctttggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 92 - protein, 5 aa, synthetic construct
  REGION 1..5: note = Description of Artificial Sequence: Synthetic peptide
NYWIT

SEQ ID NO: 93 - protein, 17 aa, synthetic construct
  REGION 1..17: note = Description of Artificial Sequence: Synthetic peptide
DFYPGGGNTNYNAKLQG

SEQ ID NO: 94 - protein, 10 aa, synthetic construct
  REGION 1..10: note = Description of Artificial Sequence: Synthetic peptide
SPPQVAPFDY

SEQ ID NO: 95 - protein, 7 aa, synthetic construct
  REGION 1..7: note = Description of Artificial Sequence: Synthetic peptide
GYTFTNY

SEQ ID NO: 96 - protein, 6 aa, synthetic construct
  REGION 1..6: note = Description of Artificial Sequence: Synthetic peptide
YPGGGN

SEQ ID NO: 97 - protein, 10 aa, synthetic construct
  REGION 1..10: note = Description of Artificial Sequence: Synthetic peptide
SPPQVAPFDY

SEQ ID NO: 98 - protein, 119 aa, synthetic construct
  REGION 1..119: note = Description of Artificial Sequence: Synthetic polypeptide
QVQLVQSGAEVKKPGASVKVSCKASGYTFTNYWITWVRQAPGQGLEWMGDFYPGGGNTNYNAKLQGRVTLTVDTSTSTAYMELRSLRSDDTAVYYCARSPPQVAPFDYWGQGTLVTVSS

SEQ ID NO: 99 - DNA, 357 bp, synthetic construct
  misc_feature 1..357: note = Description of Artificial Sequence: Synthetic polynucleotide
  source 1..357: mol_type = other DNA
caggtgcagctggtgcagtctggcgccgaagtgaagaaaccaggcgccagcgtgaaggtgtcctgcaaggccagcggctacacctttaccaactactggatcacctgggtgcgccaggcccctggacagggactggaatggatgggcgacttctaccctggcggcggcaacaccaactacaacgccaagctgcagggcagagtgaccctgaccgtggacaccagcacctccaccgcctacatggaactgcggagcctgagaagcgacgacaccgccgtgtattactgcgctagaagccctcctcaggtggcccccttcgattattggggccagggcacactcgtgaccgtgtcctct

SEQ ID NO: 100 - protein, 449 aa, synthetic construct
  REGION 1..449: note = Description of Artificial Sequence: Synthetic polypeptide
QVQLVQSGAEVKKPGASVKVSCKASGYTFTNYWITWVRQAPGQGLEWMGDFYPGGGNTNYNAKLQGRVTLTVDTSTSTAYMELRSLRSDDTAVYYCARSPPQVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1347 bp, synthetic construct
  misc_feature 1..1347: note = Description of Artificial Sequence: Synthetic polynucleotide
  source 1..1347: mol_type = other DNA
caggtgcagctggtgcagtctggcgccgaagtgaagaaaccaggcgccagcgtgaaggtgtcctgcaaggccagcggctacacctttaccaactactggatcacctgggtgcgccaggcccctggacagggactggaatggatgggcgacttctaccctggcggcggcaacaccaactacaacgccaagctgcagggcagagtgaccctgaccgtggacaccagcacctccaccgcctacatggaactgcggagcctgagaagcgacgacaccgccgtgtattactgcgctagaagccctcctcaggtggcccccttcgattattggggccagggcacactcgtgaccgtgtcctctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 102 - protein, 11 aa, synthetic construct
  REGION 1..11: note = Description of Artificial Sequence: Synthetic peptide
QASEYIYNWLG

SEQ ID NO: 103 - protein, 7 aa, synthetic construct
  REGION 1..7: note = Description of Artificial Sequence: Synthetic peptide
GASGLET

SEQ ID NO: 104 - protein, 9 aa, synthetic construct
  REGION 1..9: note = Description of Artificial Sequence: Synthetic peptide
QQYWSTPWT

SEQ ID NO: 105 - protein, 7 aa, synthetic construct
  REGION 1..7: note = Description of Artificial Sequence: Synthetic peptide
SEYIYNW

SEQ ID NO: 106 - skipped

SEQ ID NO: 107 - protein, 6 aa, synthetic construct
  REGION 1..6: note = Description of Artificial Sequence: Synthetic peptide
YWSTPW

SEQ ID NO: 108 - protein, 107 aa, synthetic construct
  REGION 1..107: note = Description of Artificial Sequence: Synthetic polypeptide
DIQMTQSPSSLSASVGDRVTITCQASEYIYNWLGWYQQKPGKAPKLLISGASGLETGVPSRFSGSGSGKDYTFTISSLQPEDIATYYCQQYWSTPWTFGQGTKLEIK

SEQ ID NO: 109 - DNA, 321 bp, synthetic construct
  misc_feature 1..321: note = Description of Artificial Sequence: Synthetic polynucleotide
  source 1..321: mol_type = other DNA
gacatccagatgacccagagccccagcagcctgtctgccagcgtgggcgacagggtgaccatcacctgtcaggccagcgaatacatctacaactggctgggctggtatcagcagaagcccggcaaggcccccaagctgctgattagcggagcctccggtctggaaaccggcgtgccaagcagattttccggcagcggctccggcaaggactacaccttcaccatcagctccctgcagcccgaggatatcgccacctactactgccagcagtactggtccaccccctggacctttggccagggcaccaagctggaaatcaag

SEQ ID NO: 110 - protein, 214 aa, synthetic construct
  REGION 1..214: note = Description of Artificial Sequence: Synthetic polypeptide
DIQMTQSPSSLSASVGDRVTITCQASEYIYNWLGWYQQKPGKAPKLLISGASGLETGVPSRFSGSGSGKDYTFTISSLQPEDIATYYCQQYWSTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - DNA, 642 bp, synthetic construct
  misc_feature 1..642: note = Description of Artificial Sequence: Synthetic polynucleotide
  source 1..642: mol_type = other DNA
gacatccagatgacccagagccccagcagcctgtctgccagcgtgggcgacagggtgaccatcacctgtcaggccagcgaatacatctacaactggctgggctggtatcagcagaagcccggcaaggcccccaagctgctgattagcggagcctccggtctggaaaccggcgtgccaagcagattttccggcagcggctccggcaaggactacaccttcaccatcagctccctgcagcccgaggatatcgccacctactactgccagcagtactggtccaccccctggacctttggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 112 - protein, 5 aa, synthetic construct
  REGION 1..5: note = Description of Artificial Sequence: Synthetic peptide
SFWIT

SEQ ID NO: 113 - protein, 17 aa, synthetic construct
  REGION 1..17: note = Description of Artificial Sequence: Synthetic peptide
DIYPGGATTNYNEKLQG

SEQ ID NO: 114 - protein, 10 aa, synthetic construct
  REGION 1..10: note = Description of Artificial Sequence: Synthetic peptide
SPPQVGPFDY

SEQ ID NO: 115 - protein, 7 aa, synthetic construct
  REGION 1..7: note = Description of Artificial Sequence: Synthetic peptide
GYTFTSF

SEQ ID NO: 116 - protein, 6 aa, synthetic construct
  REGION 1..6: note = Description of Artificial Sequence: Synthetic peptide
YPGGAT

SEQ ID NO: 117 - protein, 10 aa, synthetic construct
  REGION 1..10: note = Description of Artificial Sequence: Synthetic peptide
SPPQVGPFDY

SEQ ID NO: 118 - protein, 119 aa, synthetic construct
  REGION 1..119: note = Description of Artificial Sequence: Synthetic polypeptide
QVQLVQSGAEVKKPGASVKVSCKASGYTFTSFWITWVRQAPGQGLEWMGDIYPGGATTNYNEKLQGRVTLTVDTSTSTAYMELRSLRSDDTAVYYCARSPPQVGPFDYWGQGTLVTVSS

SEQ ID NO: 119 - DNA, 357 bp, synthetic construct
  misc_feature 1..357: note = Description of Artificial Sequence: Synthetic polynucleotide
  source 1..357: mol_type = other DNA
caggtgcagctggtgcagtctggcgccgaagtgaagaaaccaggcgccagcgtgaaggtgtcctgcaaggccagcggctataccttcaccagcttttggatcacctgggtgcgccaggcccctggacagggactggaatggatgggcgacatctaccctggcggcgccaccaccaactacaacgagaagctgcagggcagagtgaccctgaccgtggacaccagcacctccaccgcctacatggaactgcggagcctgagaagcgacgacaccgccgtgtactactgcgctagaagccctcctcaggtgggccccttcgattattggggccagggcacactcgtgaccgtgtcctct

SEQ ID NO: 120 - protein, 449 aa, synthetic construct
  REGION 1..449: note = Description of Artificial Sequence: Synthetic polypeptide
QVQLVQSGAEVKKPGASVKVSCKASGYTFTSFWITWVRQAPGQGLEWMGDIYPGGATTNYNEKLQGRVTLTVDTSTSTAYMELRSLRSDDTAVYYCARSPPQVGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - DNA, 1347 bp, synthetic construct
  misc_feature 1..1347: note = Description of Artificial Sequence: Synthetic polynucleotide
  source 1..1347: mol_type = other DNA
caggtgcagctggtgcagtctggcgccgaagtgaagaaaccaggcgccagcgtgaaggtgtcctgcaaggccagcggctataccttcaccagcttttggatcacctgggtgcgccaggcccctggacagggactggaatggatgggcgacatctaccctggcggcgccaccaccaactacaacgagaagctgcagggcagagtgaccctgaccgtggacaccagcacctccaccgcctacatggaactgcggagcctgagaagcgacgacaccgccgtgtactactgcgctagaagccctcctcaggtgggccccttcgattattggggccagggcacactcgtgaccgtgtcctctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 122 - protein, 11 aa, synthetic construct
  REGION 1..11: note = Description of Artificial Sequence: Synthetic peptide
QASDYIYHWLA

SEQ ID NO: 123 - protein, 7 aa, synthetic construct
  REGION 1..7: note = Description of Artificial Sequence: Synthetic peptide
GASSLET

SEQ ID NO: 124 - protein, 9 aa, synthetic construct
  REGION 1..9: note = Description of Artificial Sequence: Synthetic peptide
QQYWSIPWT

SEQ ID NO: 125 - protein, 7 aa, synthetic construct
  REGION 1..7: note = Description of Artificial Sequence: Synthetic peptide
SDYIYHW

SEQ ID NO: 126 - skipped

SEQ ID NO: 127 - protein, 6 aa, synthetic construct
  REGION 1..6: note = Description of Artificial Sequence: Synthetic peptide
YWSIPW

SEQ ID NO: 128 - protein, 107 aa, synthetic construct
  REGION 1..107: note = Description of Artificial Sequence: Synthetic polypeptide
DIQMTQSPSSLSASVGDRVTITCQASDYIYHWLAWYQQKPGKAPKLLISGASSLETGVPSRFSGSGSGKDYTFTISSLQPEDIATYYCQQYWSIPWTFGQGTKLEIK

SEQ ID NO: 129 - DNA, 321 bp, synthetic construct
  misc_feature 1..321: note = Description of Artificial Sequence: Synthetic polynucleotide
  source 1..321: mol_type = other DNA
gacatccagatgacccagagccccagcagcctgtctgccagcgtgggcgacagagtgaccatcacctgtcaggccagcgactacatctaccactggctggcctggtatcagcagaagcccggcaaggcccccaagctgctgattagcggagcctccagtctggaaaccggcgtgccaagcagattttccggcagcggctccggcaaggactacaccttcaccatcagctccctgcagcccgaggatatcgccacctactactgccagcagtactggtccatcccctggacctttggccagggcaccaagctggaaatcaag

SEQ ID NO: 130 - protein, 214 aa, synthetic construct
  REGION 1..214: note = Description of Artificial Sequence: Synthetic polypeptide
DIQMTQSPSSLSASVGDRVTITCQASDYIYHWLAWYQQKPGKAPKLLISGASSLETGVPSRFSGSGSGKDYTFTISSLQPEDIATYYCQQYWSIPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - DNA, 642 bp, synthetic construct
  misc_feature 1..642: note = Description of Artificial Sequence: Synthetic polynucleotide
  source 1..642: mol_type = other DNA
gacatccagatgacccagagccccagcagcctgtctgccagcgtgggcgacagagtgaccatcacctgtcaggccagcgactacatctaccactggctggcctggtatcagcagaagcccggcaaggcccccaagctgctgattagcggagcctccagtctggaaaccggcgtgccaagcagattttccggcagcggctccggcaaggactacaccttcaccatcagctccctgcagcccgaggatatcgccacctactactgccagcagtactggtccatcccctggacctttggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 132 - protein, 5 aa, synthetic construct
  REGION 1..5: note = Description of Artificial Sequence: Synthetic peptide
SFWIT

SEQ ID NO: 133 - protein, 17 aa, synthetic construct
  REGION 1..17: note = Description of Artificial Sequence: Synthetic peptide
DIYPGGANTNYNEKLQG

SEQ ID NO: 134 - protein, 10 aa, synthetic construct
  REGION 1..10: note = Description of Artificial Sequence: Synthetic peptide
SPPQVGPFDY

SEQ ID NO: 135 - protein, 7 aa, synthetic construct
  REGION 1..7: note = Description of Artificial Sequence: Synthetic peptide
GYTFTSF

SEQ ID NO: 136 - protein, 6 aa, synthetic construct
  REGION 1..6: note = Description of Artificial Sequence: Synthetic peptide
YPGGAN

SEQ ID NO: 137 - protein, 10 aa, synthetic construct
  REGION 1..10: note = Description of Artificial Sequence: Synthetic peptide
SPPQVGPFDY

SEQ ID NO: 138 - protein, 119 aa, synthetic construct
  REGION 1..119: note = Description of Artificial Sequence: Synthetic polypeptide
QVQLVQSGAEVKKPGASVKVSCKASGYTFTSFWITWVRQAPGQGLEWMGDIYPGGANTNYNEKLQGRVTLTVDTSTSTAYMELRSLRSDDTAVYYCARSPPQVGPFDYWGQGTLVTVSS

SEQ ID NO: 139 - DNA, 357 bp, synthetic construct
  misc_feature 1..357: note = Description of Artificial Sequence: Synthetic polynucleotide
  source 1..357: mol_type = other DNA
caggtgcagctggtgcagtctggcgccgaagtgaagaaaccaggcgccagcgtgaaggtgtcctgcaaggccagcggctataccttcaccagcttttggatcacctgggtgcgccaggcccctggacagggactggaatggatgggcgacatctaccctggcggcgccaacaccaactacaacgagaagctgcagggcagagtgaccctgaccgtggacaccagcacctccaccgcctacatggaactgcggagcctgagaagcgacgacaccgccgtgtactactgcgctagaagccctcctcaggtgggccccttcgattattggggccagggcacactcgtgaccgtgtcctct

SEQ ID NO: 140 - protein, 449 aa, synthetic construct
  REGION 1..449: note = Description of Artificial Sequence: Synthetic polypeptide
QVQLVQSGAEVKKPGASVKVSCKASGYTFTSFWITWVRQAPGQGLEWMGDIYPGGANTNYNEKLQGRVTLTVDTSTSTAYMELRSLRSDDTAVYYCARSPPQVGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 1347 bp, synthetic construct
  misc_feature 1..1347: note = Description of Artificial Sequence: Synthetic polynucleotide
  source 1..1347: mol_type = other DNA
caggtgcagctggtgcagtctggcgccgaagtgaagaaaccaggcgccagcgtgaaggtgtcctgcaaggccagcggctataccttcaccagcttttggatcacctgggtgcgccaggcccctggacagggactggaatggatgggcgacatctaccctggcggcgccaacaccaactacaacgagaagctgcagggcagagtgaccctgaccgtggacaccagcacctccaccgcctacatggaactgcggagcctgagaagcgacgacaccgccgtgtactactgcgctagaagccctcctcaggtgggccccttcgattattggggccagggcacactcgtgaccgtgtcctctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 142 - protein, 11 aa, synthetic construct
  REGION 1..11: note = Description of Artificial Sequence: Synthetic peptide
QASEYIINWLA

SEQ ID NO: 143 - protein, 7 aa, synthetic construct
  REGION 1..7: note = Description of Artificial Sequence: Synthetic peptide
GATGLET

SEQ ID NO: 144 - protein, 9 aa, synthetic construct
  REGION 1..9: note = Description of Artificial Sequence: Synthetic peptide
QQYWSIPWT

SEQ ID NO: 145 - protein, 7 aa, synthetic construct
  REGION 1..7: note = Description of Artificial Sequence: Synthetic peptide
SEYIINW

SEQ ID NO: 146 - skipped

SEQ ID NO: 147 - protein, 6 aa, synthetic construct
  REGION 1..6: note = Description of Artificial Sequence: Synthetic peptide
YWSIPW

SEQ ID NO: 148 - protein, 107 aa, synthetic construct
  REGION 1..107: note = Description of Artificial Sequence: Synthetic polypeptide
DIQMTQSPSSLSASVGDRVTITCQASEYIINWLAWYQQKPGKAPKLLISGATGLETGVPSRFSGSGSGKDYTFTISSLQPEDIATYYCQQYWSIPWTFGQGTKLEIK

SEQ ID NO: 149 - DNA, 321 bp, synthetic construct
  misc_feature 1..321: note = Description of Artificial Sequence: Synthetic polynucleotide
  source 1..321: mol_type = other DNA
gacatccagatgacccagagccccagcagcctgtctgccagcgtgggcgacagagtgaccatcacctgtcaggccagcgaatacatcataaactggctggcctggtatcagcagaagcccggcaaggcccccaagctgctgattagcggagccaccggtctggaaaccggcgtgccaagcagattttccggcagcggctccggcaaggactacaccttcaccatcagctccctgcagcccgaggatatcgccacctactactgccagcagtactggtccatcccctggacctttggccagggcaccaagctggaaatcaag

SEQ ID NO: 150 - protein, 214 aa, synthetic construct
  REGION 1..214: note = Description of Artificial Sequence: Synthetic polypeptide
DIQMTQSPSSLSASVGDRVTITCQASEYIINWLAWYQQKPGKAPKLLISGATGLETGVPSRFSGSGSGKDYTFTISSLQPEDIATYYCQQYWSIPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 642 bp, synthetic construct
  misc_feature 1..642: note = Description of Artificial Sequence: Synthetic polynucleotide
  source 1..642: mol_type = other DNA
gacatccagatgacccagagccccagcagcctgtctgccagcgtgggcgacagagtgaccatcacctgtcaggccagcgaatacatcataaactggctggcctggtatcagcagaagcccggcaaggcccccaagctgctgattagcggagccaccggtctggaaaccggcgtgccaagcagattttccggcagcggctccggcaaggactacaccttcaccatcagctccctgcagcccgaggatatcgccacctactactgccagcagtactggtccatcccctggacctttggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 152 - protein, 24 aa, Homo sapiens
MKTFILLLWVLLLWVIFLLPGATA

SEQ ID NO: 153 - protein, 9 aa, synthetic construct
  REGION 1..9: note = Description of Artificial Sequence: Synthetic peptide
DYKDDDDKH

SEQ ID NO: 154 - protein, 6 aa, synthetic construct
  REGION 1..6: note = Description of Artificial Sequence: Synthetic 6xHis tag
HHHHHH

SEQ ID NO: 155 - protein, 17 aa, synthetic construct
  REGION 1..17: note = Description of Artificial Sequence: Synthetic peptide
GGGLNDIFEAQKIEWHE

SEQ ID NO: 156 - protein, 438 aa, synthetic construct
  REGION 1..438: note = Description of Artificial Sequence: Synthetic polypeptide
MKTFILLLWVLLLWVIFLLPGATAQPGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAASDYKDDDDKHHHHHHGGGLNDIFEAQKIEWHE

SEQ ID NO: 157 - protein, 232 aa, synthetic construct
  REGION 1..232: note = Description of Artificial Sequence: Synthetic polypeptide
MKTFILLLWVLLLWVIFLLPGATAQPGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDYKDDDDKHHHHHHDYKDDDDKHHHHHHGGGLNDIFEAQKIEWHE

SEQ ID NO: 158 - DNA, 19 bp, synthetic construct
  misc_feature 1..19: note = Description of Artificial Sequence: Synthetic primer
  source 1..19: mol_type = other DNA
atccccgctcccaggctac

SEQ ID NO: 159 - DNA, 24 bp, synthetic construct
  misc_feature 1..24: note = Description of Artificial Sequence: Synthetic primer
  source 1..24: mol_type = other DNA
cagcaaggagtgaagctccatgcc

SEQ ID NO: 160 - protein, 438 aa, synthetic construct
  REGION 1..438: note = Description of Artificial Sequence: Synthetic polypeptide
MKTFILLLWVLLLWVIFLLPGATAQPGPVQSKSPRFASWDEMNVLAHGLLQLGQGLREHAERTRSQLNALERRLSACGSACQGTEGSTALPLAPESRVDPEVLHSLQTQLKAQNSRIQQLFHKVAQQQRHLEKQHLRIQRLQSQVGLLDPKHLDHEVAKPARRKRRPEMAQPVDSAHNASRLHRLPRDCQELFEDGERQSGLFEIQPQGSPPFLVNCKMTSDGGWTVIQRRHDGSVDFNRPWEAYKAGFGDPQGEFWLGLEKVHSITGDRNSRLAVQLQDWDGNAESLQFSVHLGGEDTAYSLQLTEPVASQLGATTVPPSGLSVPFSTWDQDHDLRRDKNCAKSLSGGWWFGTCSHSNLNGQYFRSIPQQRQELKKGIFWKTWRGRYYPLQATTMLIQPTAAEAASDYKDDDDKHHHHHHGGGLNDIFEAQKIEWHE

SEQ ID NO: 161 - protein, 218 aa, synthetic construct
  REGION 1..218: note = Description of Artificial Sequence: Synthetic polypeptide
MKTFILLLWVLLLWVIFLLPGATAQPGPVQSKSPRFASWDEMNVLAHGLLQLGQGLREHAERTRSQLNALERRLSACGSACQGTEGSTALPLAPESRVDPEVLHSLQTQLKAQNSRIQQLFHKVAQQQRHLEKQHLRIQRLQSQVGLLDPKHLDHEVAKPARRKRRPEMAQPVDSAHNASRLHRLPRDYKDDDDKHHHHHHGGGLNDIFEAQKIEWHE

SEQ ID NO: 162 - protein, 442 aa, synthetic construct
  REGION 1..442: note = Description of Artificial Sequence: Synthetic polypeptide
MKTFILLLWVLLLWVIFLLPGATAQPRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAASDYKDDDDKHHHHHHGGGLNDIFEAQKIEWHE

SEQ ID NO: 163 - protein, 440 aa, synthetic construct
  REGION 1..440: note = Description of Artificial Sequence: Synthetic polypeptide
MKTFILLLWVLLLWVIFLLPGATAQPQGRPAQPEPPRFASWDEMNLLAHGLLQLGHGLREHVERTRGQLGALERRMAACGNACQGPKGTDPKDRVPEGQAPETLQSLQTQLKAQNSKIQQLFQKVAQQQRYLSKQNLRIQNLQSQIDLLTPTHLDNGVDKTSRGKRLPKMAQLIGLTPNATRLHRPPRDCQELFQEGERHSGLFQIQPLGSPPFLVNCEMTSDGGWTVIQRRLNGSVDFNQSWEAYKDGFGDPQGEFWLGLEKMHSITGDRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQQRKKGIFWKTWKGRYYPLQATTLLIQPMEATAASDYKDDDDKHHHHHHGGGLNDIFEAQKIEWHET

SEQ ID NO: 164 - protein, 501 aa, synthetic construct
  REGION 1..501: note = Description of Artificial Sequence: Synthetic polypeptide
MKTFILLLWVLLLWVIFLLPGATAQPSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFEDYKDDDDKHHHHHHGGGLNDIFEAQKIEWHE

SEQ ID NO: 165 - protein, 10 aa, Homo sapiens
GPVQSKSPRF

SEQ ID NO: 166 - protein, 11 aa, Homo sapiens
VQSKSPRFASW

SEQ ID NO: 167 - protein, 8 aa, Homo sapiens
NVLAHGLL

SEQ ID NO: 168 - protein, 10 aa, Homo sapiens
NVLAHGLLQL

SEQ ID NO: 169 - protein, 8 aa, Homo sapiens
LAHGLLQL

SEQ ID NO: 170 - protein, 13 aa, Homo sapiens
NVLAHGLLQLGQG

SEQ ID NO: 171 - protein, 14 aa, Homo sapiens
NVLAHGLLQLGQGL

SEQ ID NO: 172 - protein, 16 aa, Homo sapiens
NVLAHGLLQLGQGLRE

SEQ ID NO: 173 - protein, 25 aa, Homo sapiens
NVLAHGLLQLGQGLREHAERTRSQL

SEQ ID NO: 174 - protein, 27 aa, Homo sapiens
NVLAHGLLQLGQGLREHAERTRSQLSA

SEQ ID NO: 175 - protein, 23 aa, Homo sapiens
LAHGLLQLGQGLREHAERTRSQL

SEQ ID NO: 176 - protein, 13 aa, Homo sapiens
AHGLLQLGQGLRE

SEQ ID NO: 177 - protein, 22 aa, Homo sapiens
AHGLLQLGQGLREHAERTRSQL

SEQ ID NO: 178 - protein, 18 aa, Homo sapiens
LQLGQGLREHAERTRSQL

SEQ ID NO: 179 - protein, 15 aa, Homo sapiens
GQGLREHAERTRSQL

SEQ ID NO: 180 - protein, 32 aa, Homo sapiens
LERRLSACGSACQTEGSTDLPAPESRVDPEVL

SEQ ID NO: 181 - protein, 25 aa, Homo sapiens
CGSACQTEGSTDLPAPESRVDPEVL

SEQ ID NO: 182 - protein, 16 aa, Homo sapiens
STDLPAPESRVDPEVL

SEQ ID NO: 183 - protein, 7 aa, Homo sapiens
RVDPEVL

SEQ ID NO: 184 - protein, 7 aa, Homo sapiens
HSLQTQL

SEQ ID NO: 185 - protein, 10 aa, Homo sapiens
KAQNSRIQQL

SEQ ID NO: 186 - protein, 26 aa, Homo sapiens
KAQNSRIQQLFHKVAQQQRHLEKQHL

SEQ ID NO: 187 - protein, 16 aa, Homo sapiens
FHKVAQQQRHLEKQHL

SEQ ID NO: 188 - protein, 7 aa, Homo sapiens
QSQFGLL

SEQ ID NO: 189 - protein, 12 aa, Homo sapiens
FGLLDHKHLDHE

SEQ ID NO: 190 - protein, 26 aa, Homo sapiens
LERRLSACGSACQTEGSTDLPLAPES

SEQ ID NO: 191 - protein, 15 aa, synthetic construct
  REGION 1..15: note = Description of Artificial Sequence: Synthetic peptide
GPVQSKSPRFASWDE

SEQ ID NO: 192 - protein, 15 aa, synthetic construct
  REGION 1..15: note = Description of Artificial Sequence: Synthetic peptide
QSKSPRFASWDEMNV

SEQ ID NO: 193 - protein, 15 aa, synthetic construct
  REGION 1..15: note = Description of Artificial Sequence: Synthetic peptide
SPRFASWDEMNVLAH

SEQ ID NO: 194 - protein, 15 aa, synthetic construct
  REGION 1..15: note = Description of Artificial Sequence: Synthetic peptide
FASWDEMNVLAHGLL

SEQ ID NO: 195 - protein, 15 aa, synthetic construct
  REGION 1..15: note = Description of Artificial Sequence: Synthetic peptide
WDEMNVLAHGLLQLG

SEQ ID NO: 196 - protein, 15 aa, synthetic construct
  REGION 1..15: note = Description of Artificial Sequence: Synthetic peptide
MNVLAHGLLQLGQGL

SEQ ID NO: 197 - protein, 15 aa, synthetic construct
  REGION 1..15: note = Description of Artificial Sequence: Synthetic peptide
LAHGLLQLGQGLREH

SEQ ID NO: 198 - protein, 15 aa, synthetic construct
  REGION 1..15: note = Description of Artificial Sequence: Synthetic peptide
GLLQLGQGLREHAER

SEQ ID NO: 199 - protein, 15 aa, synthetic construct
  REGION 1..15: note = Description of Artificial Sequence: Synthetic peptide
QLGQGLREHAERTRS

SEQ ID NO: 200 - protein, 15 aa, synthetic construct
  REGION 1..15: note = Description of Artificial Sequence: Synthetic peptide
QGLREHAERTRSQLS

SEQ ID NO: 201 - protein, 15 aa, synthetic construct
  REGION 1..15: note = Description of Artificial Sequence: Synthetic peptide
REHAERTRSQLSALE

SEQ ID NO: 202 - protein, 15 aa, synthetic construct
  REGION 1..15: note = Description of Artificial Sequence: Synthetic peptide
AERTRSQLSALERRL

SEQ ID NO: 203 - protein, 15 aa, synthetic construct
  REGION 1..15: note = Description of Artificial Sequence: Synthetic peptide
TRSQLSALERRLSAS

SEQ ID NO: 204 - protein, 15 aa, synthetic construct
  REGION 1..15: note = Description of Artificial Sequence: Synthetic peptide
QLSALERRLSASGSA

SEQ ID NO: 205 - protein, 15 aa, synthetic construct
  REGION 1..15: note = Description of Artificial Sequence: Synthetic peptide
ALERRLSASGSASQG

SEQ ID NO: 206 - protein, 15 aa, synthetic construct
  REGION 1..15: note = Description of Artificial Sequence: Synthetic peptide
RRLSASGSASQGTEG

SEQ ID NO: 207 - protein, 15 aa, synthetic construct
  REGION 1..15: note = Description of Artificial Sequence: Synthetic peptide
SASGSASQGTEGSTD

SEQ ID NO: 208 - protein, 15 aa, synthetic construct
  REGION 1..15: note = Description of Artificial Sequence: Synthetic peptide
GSASQGTEGSTDLPL

SEQ ID NO: 209 - protein, 15 aa, synthetic construct
  REGION 1..15: note = Description of Artificial Sequence: Synthetic peptide
SQGTEGSTDLPLAPE

SEQ ID NO: 210 - protein, 15 aa, synthetic construct
  REGION 1..15: note = Description of Artificial Sequence: Synthetic peptide
TEGSTDLPLAPESRV

SEQ ID NO: 211 - protein, 15 aa, synthetic construct
  REGION 1..15: note = Description of Artificial Sequence: Synthetic peptide
STDLPLAPESRVDPE

SEQ ID NO: 212 - protein, 15 aa, synthetic construct
  REGION 1..15: note = Description of Artificial Sequence: Synthetic peptide
LPLAPESRVDPEVLH

SEQ ID NO: 213 - protein, 15 aa, synthetic construct
  REGION 1..15: note = Description of Artificial Sequence: Synthetic peptide
APESRVDPEVLHSLQ

SEQ ID NO: 214 - protein, 15 aa, synthetic construct
  REGION 1..15: note = Description of Artificial Sequence: Synthetic peptide
SRVDPEVLHSLQTQL

SEQ ID NO: 215 - protein, 15 aa, synthetic construct
  REGION 1..15: note = Description of Artificial Sequence: Synthetic peptide
DPEVLHSLQTQLKAQ

SEQ ID NO: 216 - protein, 15 aa, synthetic construct
  REGION 1..15: note = Description of Artificial Sequence: Synthetic peptide
VLHSLQTQLKAQNSR

SEQ ID NO: 217 - protein, 15 aa, synthetic construct
  REGION 1..15: note = Description of Artificial Sequence: Synthetic peptide
SLQTQLKAQNSRIQQ

SEQ ID NO: 218 - protein, 15 aa, synthetic construct
  REGION 1..15: note = Description of Artificial Sequence: Synthetic peptide
TQLKAQNSRIQQLFH

SEQ ID NO: 219 - protein, 15 aa, synthetic construct
  REGION 1..15: note = Description of Artificial Sequence: Synthetic peptide
KAQNSRIQQLFHKVA

SEQ ID NO: 220 - protein, 15 aa, synthetic construct
  REGION 1..15: note = Description of Artificial Sequence: Synthetic peptide
NSRIQQLFHKVAQQQ

SEQ ID NO: 221 - protein, 15 aa, synthetic construct
  REGION 1..15: note = Description of Artificial Sequence: Synthetic peptide
IQQLFHKVAQQQRHL

SEQ ID NO: 222 - protein, 15 aa, synthetic construct
  REGION 1..15: note = Description of Artificial Sequence: Synthetic peptide
LFHKVAQQQRHLEKQ

SEQ ID NO: 223 - protein, 15 aa, synthetic construct
  REGION 1..15: note = Description of Artificial Sequence: Synthetic peptide
KVAQQQRHLEKQHLR

SEQ ID NO: 224 - protein, 15 aa, synthetic construct
  REGION 1..15: note = Description of Artificial Sequence: Synthetic peptide
QQQRHLEKQHLRIQH

SEQ ID NO: 225 - protein, 15 aa, synthetic construct
  REGION 1..15: note = Description of Artificial Sequence: Synthetic peptide
RHLEKQHLRIQHLQS

SEQ ID NO: 226 - protein, 15 aa, synthetic construct
  REGION 1..15: note = Description of Artificial Sequence: Synthetic peptide
EKQHLRIQHLQSQFG

SEQ ID NO: 227 - protein, 15 aa, synthetic construct
  REGION 1..15: note = Description of Artificial Sequence: Synthetic peptide
HLRIQHLQSQFGLLD

SEQ ID NO: 228 - protein, 15 aa, synthetic construct
  REGION 1..15: note = Description of Artificial Sequence: Synthetic peptide
IQHLQSQFGLLDHKH

SEQ ID NO: 229 - protein, 15 aa, synthetic construct
  REGION 1..15: note = Description of Artificial Sequence: Synthetic peptide
LQSQFGLLDHKHLDH

SEQ ID NO: 230 - protein, 15 aa, synthetic construct
  REGION 1..15: note = Description of Artificial Sequence: Synthetic peptide
QFGLLDHKHLDHEVA

SEQ ID NO: 231 - protein, 15 aa, synthetic construct
  REGION 1..15: note = Description of Artificial Sequence: Synthetic peptide
LLDHKHLDHEVAKPA

SEQ ID NO: 232 - protein, 15 aa, synthetic construct
  REGION 1..15: note = Description of Artificial Sequence: Synthetic peptide
HKHLDHEVAKPARRK

SEQ ID NO: 233 - protein, 15 aa, synthetic construct
  REGION 1..15: note = Description of Artificial Sequence: Synthetic peptide
KHLDHEVAKPARRKR

(19) United States
(12) Patent Application Publication
(10) Pub. No.: US 2025/0179156 A1
(43) Pub. Date: Jun. 5, 2025

(54) ANGIOPOIETIN-LIKE 4 ANTIBODIES AND METHODS OF USE

(71) Applicant: NOVARTIS AG, Basel (CH)

(72) Inventors: Regis Cebe, Saint-Louis (FR); Igor Splawski, Winchester, MA (US); John Trauger, Cambridge, MA (US); Andrei Igorevich Voznesensky, Arlington, MA (US)

(21) Appl. No.: 18/740,436

(22) Filed: Jun. 11, 2024

Related U.S. Application Data
(63) Continuation of application No. 17/699,925, filed on Mar. 21, 2022, now abandoned.
Application No. 17/699,925 is a division of application No. 16/750,462, filed on Jan. 23, 2020, now abandoned.
Application No. 16/750,462 is a division of application No. 15/686,454, filed on Aug. 25, 2017, now Pat. No. 10,577,411.
Application No. 15/686,454 is a division of application No. 14/819,680, filed on Aug. 6, 2015, now Pat. No. 9,771,417.
(60) Provisional application No. 62/034,409, filed on Aug. 7, 2014.

(51) Int. Cl. C07K 16/18; A61K 39/00; C07K 16/22

(52) U.S. Cl. CPC C07K 16/18; C07K 16/22; A61K 2039/505; C07K 2317/24; C07K 2317/33; C07K 2317/565; C07K 2317/71; C07K 2317/76; C07K 2317/92; C07K 2317/94

(57) ABSTRACT

The present invention relates to monoclonal antibodies binding to human angiopoietin-like 4 protein (hereinafter, sometimes referred to as “ANGPTL4”), and pharmaceutical compositions and methods of treatment comprising the same.

### RELATED APPLICATIONS

This application is a divisional of U.S. patent application Ser. No. 15/686,454, filed Aug. 25, 2017, which is a divisional of U.S. patent application Ser. No. 14/819,680, filed Aug. 6, 2015, and issued as U.S. Pat. No. 9,771,417 on Sep. 26, 2017, which claims priority to U.S. Provisional Patent Application No. 62/034,409, filed Aug. 7, 2014, the contents of which are incorporated herein by reference in their entireties.

### SEQUENCE LISTING

The instant application contains a Sequence Listing which was submitted in ASCII format via EFS-Web on Jan. 26, 2016, in U.S. patent application Ser. No. 14/819,680, and is hereby incorporated by reference in its entirety.

### BACKGROUND OF THE INVENTION

Angiopoietin-like 4 protein (ANGPTL4) is a member of the angiopoietin like family of secreted proteins. It is a homooligomeric protein, capable of forming dimers and tetramers, that is expressed by cell types including macrophages, adipose, muscle, and liver cells. ANGPTL4 is also known as hepatic fibrinogen/angiopoietin-related protein (HFARP)(Kim et al. (2000) Biochem. J. 346:603-610); PPAR gamma angiopoietin related protein (PGAR)(Yoon, et al. (2000) Mol. Cell Biol., 20:5343-5349), and fasting induced adipose factor (FIAF)(Kerten et al. (2000) J. Biol. Chem., 275:28488-28493). ANGPTL4 contains an N-terminal coiled-coil domain and a C-terminal fibrinogen (FBN)-like domain (Kim et al. (2000) Biochem. J. 346:603-610).

Lipoprotein lipase (LPL) has a central role in lipoprotein metabolism which includes the maintenance of lipoprotein levels in blood and, through tissue specific regulation of its activity. The coiled-coil region of ANGPTL4 is known to inhibit lipoprotein lipase (LPL)-mediated triglyceride (TG) clearance. Therefore, ANGPTL4 loss-of-function mutations (e.g., as seen in human subjects), genetic deletions (e.g., as seen in transgenic mice), and antibody inhibition (e.g., as seen in mice and cynomolgus monkeys) are all observed to decrease plasma triglycerides. Furthermore, ANGPTL4 antibodies are also known to activate LPL. Conversely, ANGPTL4 injection into mice produces a rapid increase in circulating triglycerides and this is at a higher rate than the injection of angiopoietin-like protein 3 (ANGPTL3)(Yoshida et al. (2002) J Lipid Res 43:1770-1772).

The anti-ANGPTL4 antibodies and antigen binding fragments described in this invention initiate, promote, or enhance activation of LPL, e.g., by blocking ANGPTL4 inhibition of LPL, thereby decreasing plasma triglycerides. These antibodies are expected to prevent and ameliorate the acute and chronic manifestations of diseases characterized by elevated triglyceride levels, e.g., primary dyslipidemia, hypertriglyceridemia, metabolic syndrome, type II diabetes, and the like.

### SUMMARY OF THE INVENTION

The present invention relates to monoclonal antibodies binding to human Angiopoietin-like 4 protein (hereinafter, sometimes referred to as “ANGPTL4”), and pharmaceutical compositions and methods of treatment comprising the same.

The isolated anti-ANGPTL4 antibodies, or antigen binding fragments, described herein bind ANGPTL4, with an equilibrium dissociation constant (K_{D}) of less than or equal to 100 pM. For example, the isolated antibodies or antigen binding fragments described herein may bind to human ANGPTL4 with a K_{D} of less than or equal to 150 nM, less than or equal to 50 nM, less than or equal to 10 nM, less than or equal to 750 pM, less than or equal to 600 pM, less than or equal to 500 pM, less than or equal to 400 pM, less than or equal to 300 pM, less than or equal to 200 pM, less than or equal to 100 pM, less than or equal to 75 pM, less than or equal to 65 pM, less than or equal to 60 pM, less than or equal to 55 pM. More specifically, the isolated antibodies or antigen binding fragments described herein may also bind human ANGPTL4 with a K_{D} of less than or equal to 45 pM, as measured by ForteBio kinetic binding assays, or less than or equal to 24 pM, as measured by solution equilibrium titration assay (SET); and may also bind cynomolgus monkey ANGPTL4 with a K_{D} of less than or equal to 87 pM, as measured by ForteBio kinetic binding assays, or less than or equal to 22 pM, as measured by SET.

The present invention relates to an isolated antibody, or antigen binding fragments thereof, that binds to human ANGPTL4. The present invention also relates to an isolated antibody, or antigen binding fragments thereof, that binds ANGPTL4 and further competes for binding with an antibody as described in Table 1. The present invention also further relates to an isolated antibody, or antigen binding fragments thereof, that binds the same epitope as an antibody as described in Table 1.

The binding affinity of isolated antibodies and antigen binding fragments described herein can be determined by solution equilibrium titration (SET). Methods for SET are known in the art and are described in further detail below. Alternatively, binding affinity of the isolated antibodies, or fragments, described herein can be determined by Biacore assay. Methods for Biacore kinetic assays are known in the art and are described in further detail below.

The isolated anti-ANGPTL4 antibodies and antigen binding fragments described herein can be used to inhibit ANGPTL4 binding to lipoprotein lipase (LPL) with an EC₅₀ of less than or equal to 100 nM, less than or equal to 50 nM, less than or equal to 35 nM, less than or equal to 25 nM, less than or equal to 10 nM, or less than or equal to 3 nM.

The isolated anti-ANGPTL4 antibodies, or antigen binding fragments thereof, may be used to reduce the levels of circulating triglycerides (TG).

The isolated anti-ANGPTL4 antibodies, or antigen binding fragments thereof, as described herein can be monoclonal antibodies, human or humanized antibodies, chimeric antibodies, single chain antibodies, Fab fragments, Fv fragments, F(ab′)₂ fragments, or scFv fragments, and/or IgG isotypes.

The isolated anti-ANGPTL4 antibodies, or antigen binding fragments thereof, as described herein can also include a framework in which an amino acid has been substituted into the antibody framework from the respective human VH or VL germline sequences.

Another aspect of the invention includes an isolated antibody or antigen binding fragments thereof having the full heavy and light chain sequences of humanized antibodies described in Table 1. More specifically, the isolated antibody or antigen binding fragments thereof can have the heavy and light chain sequences of NEG276, NEG276-LALA, NEG278, NEG310, NEG313, NEG315, NEG318, NEG319.

A further aspect of the invention includes an isolated antibody or antigen binding fragments thereof having the heavy and light chain variable domain sequences of humanized antibodies described in Table 1. More specifically, the isolated antibody or antigen binding fragment thereof can have the heavy and light chain variable domain sequences of NEG276, NEG276-LALA, NEG278, NEG310, NEG313, NEG315, NEG318, NEG319.

The invention also relates to an isolated antibody or antigen binding fragments thereof that includes a heavy chain CDR1 selected from the group consisting of SEQ ID NOs: 7, 32, 52, 72, 92, 112, and 132; a heavy chain CDR2 selected from the group consisting of SEQ ID NOs: 8, 33, 53, 73, 93, 113, and 133; and a heavy chain CDR3 selected from the group consisting of SEQ ID NOs: 9, 34, 54, 74, 94, 114, and 134, wherein the isolated antibody or antigen binding fragments thereof binds to human ANGPTL4. In another aspect, such isolated antibody or antigen binding fragments thereof further includes a light chain CDR1 selected from the group consisting of SEQ ID NOs: 17, 42, 62, 82, 102, 122, and 142; a light chain CDR2 selected from the group consisting of SEQ ID NOs: 18, 43, 63, 83, 103, 123, and 143; and a light chain CDR3 selected from the group consisting of SEQ ID NOs: 19, 44, 64, 84, 104, 124, and 144.

The invention also relates to an isolated antibody or antigen binding fragments thereof that includes a light chain CDR1 selected from the group consisting of SEQ ID NOs: 17, 42, 62, 82, 102, 122, and 142; a light chain CDR2 selected from the group consisting of SEQ ID NOs: 18, 43, 63, 83, 103, 123, and 143; and a light chain CDR3 selected from the group consisting of SEQ ID NOs: 19, 44, 64, 84, 104, 124, and 144, wherein the isolated antibody or antigen binding fragments thereof binds to human ANGPTL4.

The invention also relates to an isolated antibody or antigen binding fragments thereof that binds ANGPTL4 having HCDR1, HCDR2, and HCDR3 and LCDR1, LCDR2, and LCDR3, wherein HCDR1, HCDR2, and HCDR3 comprises SEQ ID NOs: 7, 8, and 9, and LCDR1, LCDR2, LCDR3 comprises SEQ ID NOs: 17, 18 and 19; or HCDR1, HCDR2, and HCDR3 comprises SEQ ID NOs: 32, 33, and 34 and LCDR1, LCDR2, LCDR3 comprises SEQ ID NOs: 42, 43 and 44; or HCDR1, HCDR2, and HCDR3 comprises SEQ ID NOs: 52, 53, and 54, and LCDR1, LCDR2, LCDR3 comprises SEQ ID NOs: 62, 63, and 64; or HCDR1, HCDR2, and HCDR3 comprises SEQ ID NOs: 72, 73, and 74, and LCDR1, LCDR2, LCDR3 comprises SEQ ID NOs: 82, 83, and 84; or HCDR1, HCDR2, and HCDR3 comprises SEQ ID NOs: 92, 93, and 94, and LCDR1, LCDR2, LCDR3 comprises SEQ ID NOs: 102, 103, and 104; or HCDR1, HCDR2, and HCDR3 comprises SEQ ID NOs: 112, 113, and 114, and LCDR1, LCDR2, LCDR3 comprises SEQ ID NOs: 122, 123, and 124; or HCDR1, HCDR2, and HCDR3 comprises SEQ ID NOs: 132, 133, and 134, and LCDR1, LCDR2, LCDR3 comprises SEQ ID NOs: 142, 143, and 144.

The invention also relates to an antibody or antigen binding fragment having HCDR1, HCDR2, and HCDR3 of the heavy chain variable domain of SEQ ID NOs: 13, 38, 58, 78, 98, 118, and 138, and the LCDR1, LCDR2 and LCDR3 of the light chain variable domain of SEQ ID NOs: 23, 48, 68, 88, 108, 128, and 148, as defined by Chothia. In another aspect of the invention the antibody or antigen binding fragment may have the HCDR1, HCDR2, and HCDR3 of the heavy chain variable domain sequence of SEQ ID NOs: 13, 38, 58, 78, 98, 118, and 138, and the LCDR1, LCDR2 and LCDR3 of the light chain variable domain sequence of SEQ ID NOs: 23, 48, 68, 88, 108, 128, and 148, as defined by Kabat.

In one aspect of the invention the isolated antibody or antigen binding fragments thereof includes a heavy chain variable domain sequence selected from the group consisting of SEQ ID NOs: 13, 38, 58, 78, 98, 118, and 138. The isolated antibody or antigen binding fragment further can comprise a light chain variable domain sequence wherein the heavy chain variable domain and light chain variable domain combine to form and antigen binding site for ANGPTL4. In particular the light chain variable domain sequence can be selected from SEQ ID NOs: 23, 48, 68, 88, 108, 128, and 148 wherein said isolated antibody or antigen binding fragments thereof binds ANGPTL4.

The invention also relates to an isolated antibody or antigen binding fragments thereof that includes a light chain variable domain sequence selected from the group consisting of SEQ ID NOs: 23, 48, 68, 88, 108, 128, and 148, wherein said isolated antibody or antigen binding fragments thereof binds to human ANGPTL4. The isolated antibody or antigen binding fragment may further comprise a heavy chain variable domain sequence wherein the light chain variable domain and heavy chain variable domain combine to form and antigen binding site for ANGPTL4.

In particular, the isolated antibody or antigen binding fragments thereof that binds ANGPTL4, may have heavy and light chain variable domains comprising the sequences of SEQ ID NOs: 13 and 23; 38 and 48; 58 and 68; 78 and 88; 98 and 108; 118 and 128; or 138 and 148, respectively.

The invention further relates to an isolated antibody or antigen binding fragments thereof, that includes a heavy chain variable domain having at least 90% sequence identity to a sequence selected from the group consisting of SEQ ID NOs: 13, 38, 58, 78, 98, 118, and 138, wherein said antibody binds to ANGPTL4. In one aspect, the isolated antibody or antigen binding fragments thereof also includes a light chain variable domain having at least 90% sequence identity to a sequence selected from the group consisting of SEQ ID NOs: 23, 48, 68, 88, 108, 128, and 148. In a further aspect of the invention, the isolated antibody or antigen binding fragment has an HCDR1, HCDR2, HCDR3, LCDR1, LCDR2 and LCDR3 as defined by Kabat and as described in Table 1.

The invention also relates to an isolated antibody or antigen binding fragments thereof, having a light chain variable domain having at least 90% sequence identity to a sequence selected from the group consisting of SEQ ID NOs: 23, 48, 68, 88, 108, 128, and 148, wherein said antibody binds ANGPTL4.

In another aspect of the invention, the isolated antibody, or antigen binding fragments thereof, that binds to ANGPTL4 may have a heavy chain comprising the sequence of SEQ ID NOs: 15, 28, 40, 60, 80, 100, 120, and 140. The isolated antibody can also includes a light chain that can combine with the heavy chain to form an antigen binding site to human ANGPTL4. In particular, the light chain may have a sequence comprising SEQ ID NOs: 25, 50, 70, 90, 110, 130, and 150. In particular, the isolated antibody or antigen binding fragments thereof that binds ANGPTL4, may have a heavy chain and a light chain comprising the sequences of SEQ ID NOs: 15 and 25; 28 and 25; 40 and 50; 60 and 70; 80 and 90; 100 and 110; 120 and 130; or 140 and 150, respectively.

The invention still further relates to an isolated antibody or antigen binding fragments thereof that includes a heavy chain having at least 90% sequence identity to a sequence selected from the group consisting of SEQ ID NOs: 15, 28, 40, 60, 80, 100, 120, and 140, wherein said antibody binds to ANGPTL4. In one aspect, the isolated antibody or antigen binding fragments thereof also includes a light chain having at least 90% sequence identity to a sequence selected from the group consisting of SEQ ID NOs: 25, 50, 70, 90, 110, 130, and 150.

The invention still further relates to an isolated antibody or antigen binding fragments thereof that includes a light chain having at least 90% sequence identity to a sequence selected from the group consisting of SEQ ID NOs: 25, 50, 70, 90, 110, 130, and 150, wherein said antibody binds ANGPTL4.

The invention still further relates to an isolated antibody or antigen binding fragment which competes for binding with the antibodies or antigen binding fragments described herein, e.g., with humanized antibodies NEG276, NEG276-LALA, NEG278, NEG310, NEG313, NEG315, NEG318, and NEG319. In one embodiment, the isolated antibody or antigen binding fragment of the invention is capable of inhibiting by more than 50% the binding of ANGPTL4 by a humanized antibody selected from NEG276, NEG276-LALA, NEG278, NEG310, NEG313, NEG315, NEG318, and NEG319, when the two antibodies or antigen binding fragments are present in equimolar concentrations.

In another embodiment, the isolated antibody or antigen binding fragment of the invention is capable of inhibiting by more than 80% the binding of ANGPTL4 by a humanized antibody selected from NEG276, NEG276-LALA, NEG278, NEG310, NEG313, NEG315, NEG318, and NEG319, when the two antibodies or antigen binding fragments are present in equimolar concentrations. In still other embodiments, the isolated antibody or antigen binding fragment of the invention is capable of inhibiting by more than 85% (or 90%, 95%, 98% or 99%) the binding of ANGPTL4 by a humanized antibody selected from NEG276, NEG276-LALA, NEG278, NEG310, NEG313, NEG315, NEG318, and NEG319, when the two antibodies or antigen binding fragments are present in equimolar concentrations.

The invention also relates to compositions comprising the isolated antibody, or antigen binding fragments thereof, described herein. As well as, antibody compositions in combination with a pharmaceutically acceptable carrier. Specifically, the invention further includes pharmaceutical compositions comprising an antibody or antigen binding fragments thereof of Table 1, such as, for example humanized antibodies NEG276, NEG276-LALA, NEG278, NEG310, NEG313, NEG315, NEG318, NEG319. The invention also relates to pharmaceutical compositions comprising a combination of two or more of the isolated antibodies or antigen binding fragments thereof of Table 1.

The invention also relates to an isolated nucleic acid sequence encoding the heavy chain variable domain having a sequence selected from SEQ ID NOs: 13, 38, 58, 78, 98, 118, and 138. In particular the nucleic acid has a sequence at least 90% sequence identity to a sequence selected from the group consisting of SEQ ID NOs: 14, 27, 39, 59, 79, 99, 119, and 139. In a further aspect of the invention the sequence is SEQ ID NOs: 14, 27, 39, 59, 79, 99, 119, or 139.

The invention also relates to an isolated nucleic acid sequence encoding the light chain variable domain having a sequence selected from SEQ ID NOs: 23, 48, 68, 88, 108, 128, and 148. In particular the nucleic acid has a sequence at least 90% sequence identity to a sequence selected from the group consisting of SEQ ID NOs: 24, 31, 49, 69, 89, 109, 129, and 149. In a further aspect of the invention the sequence is SEQ ID NOs: 24, 31, 49, 69, 89, 109, 129, or 149.

The invention also relates to an isolated nucleic acid comprising a sequence encoding a polypeptide that includes a light chain variable domain having at least 90% sequence identity to a sequence selected from the group consisting of SEQ ID NOs: 23, 48, 68, 88, 108, 128, and 148.

The invention also relates to a vector that includes one or more of the nucleic acid molecules described herein.

The invention also relates to an isolated host cell that includes a recombinant DNA sequence encoding a heavy chain of the antibody described above, and a second recombinant DNA sequence encoding a light chain of the antibody described above, wherein said DNA sequences are operably linked to a promoter and are capable of being expressed in the host cell. It is contemplated that the antibody can be a humanized antibody. It is also contemplated that the host cell is a non-human mammalian cell.

It is contemplated that the cell is a human cell. It is further contemplated that the cell is in a subject. In one embodiment, it is contemplated that the cell is an endothelial cell. In other embodiments, the cell may be one or more of adipose, muscle, and liver cells. It is still further contemplated that the subject is human.

The invention also relates to a method of treating, improving, or preventing a ANGPTL4-associated disorder in a patient, wherein the method includes the step of administering to the patient an effective amount of a composition comprising the antibody or antigen binding fragments thereof described herein. In one aspect, the ANGPTL4-associated disorder is associated with hypertriglyceridemia (e.g., severe hypertriglyceridemia (e.g., with plasma triglyceride concentration >500 mg/dL), hypertriglyceridemia associated with obesity, and type V hypertriglyceridemia). In other aspects, the ANGPTL4-associated disorder is associated with primary dyslipidemia, metabolic syndrome, type II diabetes. It is contemplated that the patient is human.

Any of the foregoing isolated antibodies or antigen binding fragments thereof may be a monoclonal antibody or antigen binding fragments thereof.

### Definitions

Unless defined otherwise, all technical and scientific terms used herein have the same meaning as commonly understood by those of ordinary skill in the art to which this invention pertains.

The terms “ANGPTL4 protein” or “ANGPTL4 antigen” or “ANGPTL4” are used interchangeably, and refer to the Angiopoietin-like 4 (ANGPTL4) protein in different species. For example, human ANGPTL4 has the sequence as set out in Table 1 (SEQ ID NO: 1), and has been described in previous reports and literature (Nature, Vol. 386, p. 73-77, 1997; Genomics, Vol. 54, No. 2, p. 191-199, 1998; Biochem. J., Vol. 339, Part 1, P. 177-184, 1999; Genbank Accession No. NP 002534). ANGPTL4 contains an N-terminal coiled-coil domain and a C-terminal fibrinogen (FBN)-like domain (Kim et al. (2000) Biochem. J. 346:603-610). It is a homooligomeric protein, capable of forming dimers and tetramers, that is expressed by cell types including macrophages, adipose, muscle, and liver cells, and known to inhibit lipoprotein lipase (LPL)-mediated triglyceride (TG) clearance.

In addition, in the context of this invention, the term “ANGPTL4” includes mutants of the natural Angiopoietin-like 4 (ANGPTL4) protein, which have substantially the same amino acid sequence as that of the native primary structure (amino acid sequence) described in the above-mentioned reports. Herein, the term “mutants of the natural human Angiopoietin-like 4 (ANGPTL4) protein having substantially the same amino acid sequence” refers to such mutant proteins.

The term “antibody” as used herein means a whole antibody and any antigen binding fragment (i.e., “antigen-binding portion”) or single chain thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (CIq) of the classical complement system.

The term “antigen binding portion” or “antigen binding fragment” of an antibody, as used herein, refers to one or more fragments of an intact antibody that retain the ability to specifically bind to a given antigen (e.g., human oxidized LDL receptor (ANGPTL4)). Antigen binding functions of an antibody can be performed by fragments of an intact antibody. Examples of binding fragments encompassed within the term antigen binding portion or antigen binding fragment of an antibody include a Fab fragment, a monovalent fragment consisting of the VL, VH, CL and CH1 domains; a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain or a VL domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more antigen binding portions or fragments of an antibody. These antibody fragments are obtained using conventional techniques known to those of skill in the art, and the fragments are screened for utility in the same manner as are intact antibodies.

Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3)(see U.S. Pat. No. 6,703,199, which describes fibronectin polypeptide monobodies).

Antigen binding fragments can be incorporated into single chain molecules comprising a pair of tandem Fv segments (VH-CH1-VH—CH1) which, together with complementary light chain polypeptides, form a pair of antigen binding regions (Zapata et al., 1995 Protein Eng. 8 (10): 1057-1062; and U.S. Pat. No. 5,641,870).

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. As used herein, the term “high affinity” for an antibody or antigen binding fragments thereof (e.g., a Fab fragment) generally refers to an antibody, or antigen binding fragment, having a K_{D} of 10⁻⁹ M or less.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “binding specificity” as used herein refers to the ability of an individual antibody combining site to react with only one antigenic determinant.

The phrase “specifically (or selectively) binds” to an antibody (e.g., a ANGPTL4-binding antibody) refers to a binding reaction that is determinative of the presence of a cognate antigen (e.g., a human ANGPTL4 or cynomolgus ANGPTL4) in a heterogeneous population of proteins and other biologics. The phrases “an antibody recognizing an antigen” and “an antibody specific for an antigen” are used interchangeably herein with the term “an antibody which binds specifically to an antigen.”

The term “ANGPTL4 mediated” refers to the fact that ANGPTL4 is known to inhibit lipoprotein lipase (LPL)-mediated triglyceride (TG) clearance, and thereby increase triglyceride levels.

An “ANGPTL4-associated disorder,” “ANGPTL4-associated condition,” or similar terms as used herein, refer to any number of conditions or diseases in which ANGPTL4ANGPTL4 a reduction of ANGPTL4-mediated LPL inhibition and lipoprotein modulation is sought. These conditions include but are not limited to those involving lipid metabolism, such as hyperlipidemia, hyperlipoproteinemia and dyslipidemia, including atherogenic dyslipidemia, diabetic dyslipidemia, hypertriglyceridemia (e.g., severe hypertriglyceridemia (e.g., with plasma triglyceride concentration >500 mg/dL), hypertriglyceridemia associated with obesity, and type V hypertriglyceridemia), hypercholesterolemia, chylomicronemia, mixed dyslipidemia (obesity, metabolic syndrome, diabetes, etc.), lipodystrophy, lipoatrophy, and other conditions caused by, e.g., decreased LPL activity and/or LPL deficiency, decreased LDL receptor activity and/or LDL receptor deficiency, altered ApoC2, ApoE deficiency, increased ApoB, increased production and/or decreased elimination of very low-density lipoprotein (VLDL), certain drug treatment (e.g., glucocorticoid treatment-induced dyslipidemia), any genetic predisposition, diet, life style, and the like.

Other ANGPTL4-associated diseases or disorders associated with or resulting from hyperlipidemia, hyperlipoproteinemia, and/or dyslipidemia, include, but are not limited to, cardiovascular diseases or disorders, such as atherosclerosis, aneurysm, hypertension, angina, stroke, cerebrovascular diseases, congestive heart failure, coronary artery diseases, myocardial infarction, peripheral vascular diseases, and the like; acute pancreatitis; nonalcoholic steatohepatitis (NASH); blood sugar disorders, such as diabetes; obesity, and the like.

The term “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M)(see, e.g., Creighton, Proteins (1984)). In some embodiments, the term “conservative sequence modifications” are used to refer to amino acid modifications that do not significantly affect or alter the binding characteristics of the antibody containing the amino acid sequence.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo).

A “humanized” antibody is an antibody that retains the antigen-specific reactivity of a non-human antibody, e.g., a mouse monoclonal antibody, while being less immunogenic when administered as a therapeutic in humans. See, e.g., Robello et al., Transplantation, 68:1417-1420. This can be achieved, for instance, by retaining the non-human antigen-binding regions and replacing the remaining parts of the antibody with their human counterparts (i.e., the constant region as well as portions of the variable region not involved in binding). See, e.g., Morrison et al., Proc. Natl. Acad. Sci. USA, 81:6851-6855, 1984; Morrison and Oi, Adv. Immunol., 44:65-92, 1989; Verhoeyen et al., Science, 239:1534-1536, 1988; Padlan, Molec. Immun., 28:489-498, 1991; and Padlan, Molec. Immun., 31:169-217, 1994. Other examples of human engineering technology include, but are not limited to Xoma technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., Current Protocols in Molecular Biology, John Wiley & Sons, Inc. (Ringbou ed., 2003)).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available on the world wide web at gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated antibody” refers to an antibody that is substantially free of other antibodies having different antigenic specificities (e.g., an isolated antibody that specifically binds ANGPTL4 is substantially free of antibodies that specifically bind antigens other than ANGPTL4). An isolated antibody that specifically binds ANGPTL4 may, however, have cross-reactivity to other antigens. Moreover, an isolated antibody may be substantially free of other cellular material and/or chemicals.

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors.

The term “K_{assoc}” or “kₐ”, as used herein, is intended to refer to the association rate of a particular antibody-antigen interaction, whereas the term “k_{dis}” or “k_{d},” as used herein, is intended to refer to the dissociation rate of a particular antibody-antigen interaction. The term “K_{D}”, as used herein, is intended to refer to the dissociation constant, which is obtained from the ratio of k_{d} to kₐ (i.e. k_{d}/kₐ) and is expressed as a molar concentration (M). K_{D} values for antibodies can be determined using methods well established in the art. Methods for determining the K_{D} of an antibody include measuring surface plasmon resonance using a biosensor system such as a Biacore® system, or measuring affinity in solution by solution equilibrium titration (SET).

The terms “monoclonal antibody” or “monoclonal antibody composition” as used herein refer to a preparation of antibody molecules of single molecular composition. A monoclonal antibody composition displays a single binding specificity and affinity for a particular epitope.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “operably linked” refers to a functional relationship between two or more polynucleotide (e.g., DNA) segments. Typically, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject” are used herein interchangeably. As used herein, the terms “cyno” or “cynomolgus” refer to the cynomolgus monkey (Macaca fascicularis).

As used herein, the term “treating” or “treatment” of any disease or disorder (e.g., ANGPTL4 associated disorder) refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

“Prevention” as it relates to indications described herein, including, e.g., ANGPTL4 associated disorder, means any action that prevents or slows a worsening in e.g., ANGPTL4 associated disease parameters, as described below, in a patient at risk for said worsening.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### BRIEF DESCRIPTION OF THE DRAWINGS

FIGS. 1A-1D depicts the reversal of ANGPTL4-mediated inhibition of human lipoprotein lipase (LPL) protein by selected ANGPTL4 antibodies of the invention.

FIG. 2 depicts binding of selected antibodies of the invention to full-length human ANGPTL4 and human ANGPTL4 N-terminal coiled coil domain, and absence of binding to human full-length ANGPTL3. ANGPTL3 Ab=ANGPTL3-specific reference antibody.

FIG. 3A-3B depicts changes in plasma triglyceride levels in human ANGPTL4 transgenic mice following administration of selected ANGPTL4 antibodies of the invention.

FIG. 4 depicts plasma total human antibody concentrations in obese, diabetic cynomolgus monkeys following administration of one ANGPTL4 antibody of the invention (NEG276-LALA).

FIG. 5 depicts changes in plasma triglyceride (TG) concentrations in obese, diabetic cynomolgus monkeys following administration of one ANGPTL4 antibody of the invention (NEG276-LALA).

FIG. 6 depicts changes in plasma total cholesterol concentration in obese, diabetic cynomolgus monkeys following administration of one ANGPTL4 antibody of the invention (NEG276-LALA).

FIG. 7 depicts changes in plasma high-density lipoprotein (HDL) concentrations in obese, diabetic cynomolgus monkeys following administration of one ANGPTL4 antibody of the invention (NEG276-LALA).

FIG. 8 depicts changes in plasma total apolipoprotein B (ApoB) concentrations in obese, diabetic cynomolgus monkeys following administration of one ANGPTL4 antibody of the invention (NEG276-LALA).

FIG. 9 depicts changes in plasma apolipoprotein C—III (ApoC-III) concentrations in obese, diabetic cynomolgus monkeys following administration of one ANGPTL4 antibody of the invention (NEG276-LALA).

FIG. 10 depicts changes in plasma lipoprotein-associated cholesterol levels as assessed by fast-protein liquid chromatography (FPLC) separation of plasma lipoprotein following administration of one ANGPTL4 antibody of the invention. Data from one monkey is shown (NEG276-LALA, monkey #6296). Abbreviations: TRL, triglyceride-rich lipoproteins; LDL, low-density lipoprotein; HDL, high-density lipoprotein.

FIG. 11 depicts changes in plasma lipoprotein-associated triglyceride (TG) levels as assessed by fast-protein liquid chromatography (FPLC) separation of plasma lipoprotein following administration of one ANGPTL4 antibody of the invention. Data from one monkey is shown (NEG276-LALA, monkey #6296). Abbreviations: TRL, triglyceride-rich lipoproteins; LDL, low-density lipoprotein; HDL, high-density lipoprotein.

### DETAILED DESCRIPTION

The present invention is based, in part, on the discovery of antibody molecules that specifically bind to ANGPTL4 and inhibit its biological activities. The invention relates to both full IgG format antibodies (e.g., humanized antibodies NEG276, NEG276-LALA, NEG278, NEG310, NEG313, NEG315, NEG318, NEG319) as well as antigen binding fragments thereof, such as Fab fragments.

Accordingly, the present invention provides antibodies that specifically bind to ANGPTL4 (e.g., human ANGPTL4), pharmaceutical compositions, production methods, and methods of use of such antibodies and compositions.

### ANGPTL4 Proteins

The present invention provides antibodies that specifically bind to ANGPTL4 and inhibit its biological activities, including ability to activate lipoprotein lipase (LPL). Conversely,

Angiopoietin-like 4 protein (ANGPTL4) is a member of the angiopoietin family of secreted proteins. It is a homooligomeric protein, capable of forming dimers and tetramers, that is expressed by cell types including macrophages, adipose, muscle, and liver cells. ANGPTL4 is also known as hepatic fibrinogen/angiopoietin-related protein (HFARP)(Kim et al. (2000) Biochem. J. 346:603-610); PPAR gamma angiopoietin related protein (PGAR)(Yoon, et al. (2000) Mol. Cell Biol., 20:5343-5349), and fasting induced adipose factor (FIAF)(Kerten et al. (2000) J. Biol. Chem., 275:28488-28493). ANGPTL4 contains an N-terminal coiled-coil domain and a C-terminal fibrinogen (FBN)-like domain (Kim et al. (2000) Biochem. J. 346:603-610).

Lipoprotein lipase (LPL) has a central role in lipoprotein metabolism to maintain normal lipoprotein levels in blood and, through tissue specific regulation of its activity, to determine when and in what tissues triglycerides (TG) are unloaded. The coiled-coil region of ANGPTL4 is known to inhibit lipoprotein lipase (LPL)-mediated triglyceride (TG) clearance. Therefore, ANGPTL4 loss-of-function mutations (e.g., as seen in human subjects), genetic deletions (e.g., as seen in transgenic mice), and antibody inhibition (e.g., as seen in mice and cynomolgus monkeys) are all observed to decrease plasma triglycerides. Furthermore, ANGPTL4 antibodies are also known to activate LPL. Conversely, ANGPTL4 injection into mice produces a rapid increase in circulating triglycerides and this is at a higher rate than the injection of angiopoietin-like protein 3 (ANGPTL3)(Yoshida et al. (2002) J Lipid Res 43:1770-1772).

The anti-ANGPTL4 antibodies and antigen binding fragments described in this invention initiate, promote, or enhance activation of LPL, e.g., by blocking ANGPTL4 inhibition of LPL, thereby decreasing plasma triglycerides. These antibodies are expected to prevent and ameliorate the acute and chronic manifestations of diseases characterized by elevated triglyceride levels, e.g., primary dyslipidemia, hypertriglyceridemia, metabolic syndrome, type II diabetes, and the like.

The anti-ANGPTL4 antibodies and antigen binding fragments described in this invention initiate, promote, or enhance activation of LPL, e.g., by blocking ANGPTL4 inhibition of LPL, thereby decreasing plasma triglycerides. These antibodies are expected to prevent and ameliorate the acute and chronic manifestations of diseases characterized by elevated triglyceride levels, e.g., primary dyslipidemia, hypertriglyceridemia, metabolic syndrome, type II diabetes, and the like.

### ANGPTL4 Antibodies & Antigen Binding Fragments

The present invention provides antibodies that specifically bind to ANGPTL4. In some embodiments, the present invention provides antibodies that specifically bind to human and cynomolgus monkey ANGPTL4. Antibodies of the invention include, but are not limited to, the humanized antibodies and Fabs, isolated as described in the Examples.

The present invention provides antibodies that specifically bind a ANGPTL4 protein (e.g., human and cynomolgus monkey ANGPTL4), wherein the antibodies comprise a VH domain having an amino acid sequence of SEQ ID NOs: 13, 38, 58, 78, 98, 118, and 138. The present invention also provides antibodies that specifically bind to a ANGPTL4 protein, wherein the antibodies comprise a VH CDR having an amino acid sequence of any one of the VH CDRs listed in Table 1, infra. In particular, the invention provides antibodies that specifically bind to an ANGPTL4 protein (e.g., human and cynomolgus monkey ANGPTL4), wherein the antibodies comprise (or alternatively, consist of) one, two, three, or more VH CDRs having an amino acid sequence of any of the VH CDRs listed in Table 1, infra.

The present invention provides antibodies that specifically bind to a ANGPTL4 protein, said antibodies comprising a VL domain having an amino acid sequence of SEQ ID NOs: 23, 48, 68, 88, 108, 128, and 148. The present invention also provides antibodies that specifically bind to an ANGPTL4 protein (e.g., human and cynomolgus monkey ANGPTL4), said antibodies comprising a VL CDR having an amino acid sequence of any one of the VL CDRs listed in Table 2, infra. In particular, the invention provides antibodies that specifically bind to an ANGPTL4 protein (e.g., human and cynomolgus monkey ANGPTL4), said antibodies comprising (or alternatively, consisting of) one, two, three or more VL CDRs having an amino acid sequence of any of the VL CDRs listed in Table 1, infra.

Other antibodies of the invention include amino acids that have been mutated, yet have at least 60, 70, 80, 85, 90 or 95 percent identity in the CDR regions with the CDR regions depicted in the sequences described in Table 1. In some embodiments, it includes mutant amino acid sequences wherein no more than 1, 2, 3, 4 or 5 amino acids have been mutated in the CDR regions when compared with the CDR regions depicted in the sequence described in Table 1.

The present invention also provides nucleic acid sequences that encode VH, VL, the full-length heavy chain, and the full-length light chain of the antibodies that specifically bind to an ANGPTL4 protein (e.g., human and cynomolgus monkey ANGPTL4). Such nucleic acid sequences can be optimized for expression in mammalian cells (for example, Table 1 shows the optimized nucleic acid sequences for the heavy chain and light chain of antibodies of the invention).

TABLE 1
Examples of ANGPTL4 Antibodies, Fabs and ANGPTL4 Proteins

| | Sequence | |
|---|---|---|
| Sequence | Identifier | |
| Description | (SEQ ID NO.) | Amino acid or polynucleotide sequence |
| Human ANGPTL4 | 1 | MSGAPTAGAALMLCAATAVLLSAQGGPVQSKSPRFASWDEM |
| amino acid | | NVLAHGLLQLGQGLREHAERTRSQLSALERRLSACGSACQG |
| sequence (NCBI | | TEGSTDLPLAPESRVDPEVLHSLQTQLKAQNSRIQQLFHKV |
| Reference | | AQQQRHLEKQHLRIQHLQSQFGLLDHKHLDHEVAKPARRKR |
| Sequence: | | LPEMAQPVDPAHNVSRLHRLPRDCQELFQVGERQSGLFEIQ |
| NM_139314.2) | | PQGSPPFLVNCKMTSDGGWTVIQRRHDGSVDFNRPWEAYKA |
| | | GFGDPHGEFWLGLEKVHSITGDRNSRLAVQLRDWDGNAELL |
| | | QFSVHLGGEDTAYSLQLTAPVAGQLGATTVPPSGLSVPFST |
| | | WDQDHDLRRDKNCAKSLSGGWWFGTCSHSNLNGQYFRSIPQ |
| | | QRQKLKKGIFWKTWRGRYYPLQATTMLIQPMAAEAAS |
| Human ANGPTL4 | 2 | ATGAGCGGTGCTCCGACGGCCGGGGCAGCCCTGATGCTCTG |
| nucleic acid | | CGCCGCCACCGCCGTGCTACTGAGCGCTCAGGGCGGACCCG |
| sequence (NCBI | | TGCAGTCCAAGTCGCCGCGCTTTGCGTCCTGGGACGAGATG |
| Reference | | AATGTCCTGGCGCACGGACTCCTGCAGCTCGGCCAGGGGCT |
| NM_139314.2) | | GCGCGAACACGCGGAGCGCACCCGCAGTCAGCTGAGCGCGC |
| | | TGGAGCGGCGCCTGAGCGCGTGCGGGTCCGCCTGTCAGGGA |
| | | ACCGAGGGGTCCACCGACCTCCCGTTAGCCCCTGAGAGCCG |
| | | GGTGGACCCTGAGGTCCTTCACAGCCTGCAGACACAACTCA |
| | | AGGCTCAGAACAGCAGGATCCAGCAACTCTTCCACAAGGTG |
| | | GCCCAGCAGCAGCGGCACCTGGAGAAGCAGCACCTGCGAAT |
| | | TCAGCATCTGCAAAGCCAGTTTGGCCTCCTGGACCACAAGC |
| | | ACCTAGACCATGAGGTGGCCAAGCCTGCCCGAAGAAAGAGG |
| | | CTGCCCGAGATGGCCCAGCCAGTTGACCCGGCTCACAATGT |
| | | CAGCCGCCTGCACCGGCTGCCCAGGGATTGCCAGGAGCTGT |
| | | TCCAGGTTGGGGAGAGGCAGAGTGGACTATTTGAAATCCAG |
| | | CCTCAGGGGTCTCCGCCATTTTTGGTGAACTGCAAGATGAC |
| | | CTCAGATGGAGGCTGGACAGTAATTCAGAGGCGCCACGATG |
| | | GCTCAGTGGACTTCAACCGGCCCTGGGAAGCCTACAAGGCG |
| | | GGGTTTGGGGATCCCCACGGCGAGTTCTGGCTGGGTCTGGA |
| | | GAAGGTGCATAGCATCACGGGGGACCGCAACAGCCGCCTGG |
| | | CCGTGCAGCTGCGGGACTGGGATGGCAACGCCGAGTTGCTG |
| | | CAGTTCTCCGTGCACCTGGGTGGCGAGGACACGGCCTATAG |
| | | CCTGCAGCTCACTGCACCCGTGGCCGGCCAGCTGGGCGCCA |
| | | CCACCGTCCCACCCAGCGGCCTCTCCGTACCCTTCTCCACT |
| | | TGGGACCAGGATCACGACCTCCGCAGGGACAAGAACTGCGC |
| | | CAAGAGCCTCTCTGGAGGCTGGTGGTTTGGCACCTGCAGCC |
| | | ATTCCAACCTCAACGGCCAGTACTTCCGCTCCATCCCACAG |
| | | CAGCGGCAGAAGCTTAAGAAGGGAATCTTCTGGAAGACCTG |
| | | GCGGGGCCGCTACTACCCGCTGCAGGCCACCACCATGTTGA |
| | | TCCAGCCCATGGCAGCAGAGGCAGCCTCCTAGCGTC |
| Cyno ANGPTL4 | 3 | MRGAPTAGAALMLCVATAVLLRAQGGPVQSKSPRFASWDEM |
| (amino acid | | NVLAHGLLQLGQGLREHAERTRSQLNALERRLSACGSACQG |
| sequence) | | TEGSTALPLAPESRVDPEVLHSLQTQLKAQNSRIQQLFHKV |
| | | AQQQRHLEKQHLRIQRLQSQVGLLDPKHLDHEVAKPARRKR |
| | | RPEMAQPVDSAHNASRLHRLPRDCQELFEDGERQSGLFEIQ |
| | | PQGSPPFLVNCKMTSDGGWTVIQRRHDGSVDENRPWEAYKA |
| | | GFGDPQGEFWLGLEKVHSITGDRNSRLAVQLQDWDGNAESL |
| | | QFSVHLGGEDTAYSLQLTEPVASQLGATTVPPSGLSVPFST |
| | | WDQDHDLRRDKNCAKSLSGGWWFGTCSHSNLNGQYFRSIPQ |
| | | QRQELKKGIFWKTWRGRYYPLQATTMLIQPTAAEAAS |
| Cyno ANGPTL4 | 4 | ATGCGCGGTGCTCCGACGGCCGGAGCAGCCCTGATGCTCTG |
| (nucleic acid | | CGTCGCCACGGCCGTGCTGCTGAGAGCTCAGGGCGGCCCGG |
| sequence) | | TGCAGTCCAAGTCTCCGCGCTTTGCGTCCTGGGACGAGATG |
| | | AATGTCCTGGCGCACGGACTCCTGCAGCTAGGCCAGGGGCT |
| | | GCGCGAACACGCGGAGCGCACCCGCAGTCAGCTGAACGCGC |
| | | TGGAGCGGCGCCTCAGCGCTTGCGGGTCTGCCTGCCAGGGA |
| | | ACCGAGGGGTCCACCGCCCTCCCGTTAGCCCCTGAGAGCCG |
| | | GGTGGACCCTGAGGTCCTTCACAGCCTGCAGACACAACTCA |
| | | AGGCTCAGAACAGCAGGATCCAGCAACTCTTCCACAAGGTG |
| | | GCCCAGCAGCAGCGGCACCTGGAGAAGCAGCACCTGCGAAT |
| | | TCAGCGTCTGCAAAGCCAGGTTGGCCTCCTGGACCCCAAGC |
| | | ACCTAGACCATGAGGTGGCCAAGCCTGCCCGAAGAAAGAGG |
| | | CGGCCCGAGATGGCCCAGCCAGTTGACTCGGCTCACAATGC |
| | | CAGCCGCCTGCACCGGCTGCCCAGGGATTGCCAGGAGCTGT |
| | | TTGAAGATGGGGAGAGGCAGAGTGGACTATTTGAGATCCAG |
| | | CCTCAGGGGTCTCCGCCATTTTTGGTGAACTGCAAGATGAC |
| | | CTCAGATGGAGGCTGGACAGTAATTCAGAGGCGCCACGATG |
| | | GCTCTGTGGACTTCAACCGGCCCTGGGAAGCCTACAAGGCG |
| | | GGGTTTGGGGATCCCCAAGGCGAGTTCTGGCTGGGCCTGGA |
| | | GAAGGTGCATAGCATCACAGGGGACCGCAACAGCCGCCTGG |
| | | CCGTGCAGCTGCAGGACTGGGATGGCAACGCCGAGTCGCTG |
| | | CAGTTCTCTGTGCACCTGGGTGGCGAGGACACGGCTTACAG |
| | | CCTGCAGCTCACCGAGCCCGTGGCCAGCCAGTTGGGTGCCA |
| | | CCACCGTCCCGCCTAGCGGCCTCTCCGTACCCTTCTCCACT |
| | | TGGGACCAGGATCACGACCTCCGCAGGGACAAGAACTGCGC |
| | | CAAGAGCCTCTCTGGAGGCTGGTGGTTTGGCACCTGCAGCC |
| | | ATTCCAACCTCAATGGCCAGTACTTCCGCTCCATCCCACAG |
| | | CAGCGGCAGGAGCTTAAGAAAGGAATCTTCTGGAAGACCTG |
| | | GCGGGGCCGCTACTACCCGCTGCAGGCCACCACCATGTTGA |
| | | TCCAGCCCACGGCGGCAGAGGCAGCCTCCTAG |
| Human ANGPTL3 | 5 | MFTIKLLLFIVPLVISSRIDQDNSSFDSLSPEPKSRFAMLD |
| amino acid | | DVKILANGLLQLGHGLKDFVHKTKGQINDIFQKLNIFDQSF |
| sequence (NCBI | | YDLSLQTSEIKEEEKELRRTTYKLQVKNEEVKNMSLELNSK |
| Reference | | LESLLEEKILLQQKVKYLEEQLTNLIQNQPETPEHPEVTSL |
| NM_014495.3) | | KTFVEKQDNSIKDLLQTVEDQYKQLNQQHSQIKEIENQLRR |
| | | TSIQEPTEISLSSKPRAPRTTPFLQLNEIRNVKHDGIPAEC |
| | | TTIYNRGEHTSGMYAIRPSNSQVFHVYCDVISGSPWTLIQH |
| | | RIDGSQNFNETWENYKYGFGRLDGEFWLGLEKIYSIVKQSN |
| | | YVLRIELEDWKDNKHYIEYSFYLGNHETNYTLHLVAITGNV |
| | | PNAIPENKDLVESTWDHKAKGHENCPEGYSGGWWWHDECGE |
| | | NNLNGKYNKPRAKSKPERRRGLSWKSQNGRLYSIKSTKMLI |
| | | HPTDSESFE |
| Human ANGPTL3 | 6 | ATGTTCACAATTAAGCTCCTTCTTTTTATTGTTCCTCTAGT |
| nucleic acid | | TATTTCCTCCAGAATTGATCAAGACAATTCATCATTTGATT |
| sequence (NCBI | | CTCTATCTCCAGAGCCAAAATCAAGATTTGCTATGTTAGAC |
| Reference | | GATGTAAAAATTTTAGCCAATGGCCTCCTTCAGTTGGGACA |
| NM_014495.3) | | TGGTCTTAAAGACTTTGTCCATAAGACGAAGGGCCAAATTA |
| | | ATGACATATTTCAAAAACTCAACATATTTGATCAGTCTTTT |
| | | TATGATCTATCGCTGCAAACCAGTGAAATCAAAGAAGAAGA |
| | | AAAGGAACTGAGAAGAACTACATATAAACTACAAGTCAAAA |
| | | ATGAAGAGGTAAAGAATATGTCACTTGAACTCAACTCAAAA |
| | | CTTGAAAGCCTCCTAGAAGAAAAAATTCTACTTCAACAAAA |
| | | AGTGAAATATTTAGAAGAGCAACTAACTAACTTAATTCAAA |
| | | ATCAACCTGAAACTCCAGAACACCCAGAAGTAACTTCACTT |
| | | AAAACTTTTGTAGAAAAACAAGATAATAGCATCAAAGACCT |
| | | TCTCCAGACCGTGGAAGACCAATATAAACAATTAAACCAAC |
| | | AGCATAGTCAAATAAAAGAAATAGAAAATCAGCTCAGAAGG |
| | | ACTAGTATTCAAGAACCCACAGAAATTTCTCTATCTTCCAA |
| | | GCCAAGAGCACCAAGAACTACTCCCTTTCTTCAGTTGAATG |
| | | AAATAAGAAATGTAAAACATGATGGCATTCCTGCTGAATGT |
| | | ACCACCATTTATAACAGAGGTGAACATACAAGTGGCATGTA |
| | | TGCCATCAGACCCAGCAACTCTCAAGTTTTTCATGTCTACT |
| | | GTGATGTTATATCAGGTAGTCCATGGACATTAATTCAACAT |
| | | CGAATAGATGGATCACAAAACTTCAATGAAACGTGGGAGAA |
| | | CTACAAATATGGTTTTGGGAGGCTTGATGGAGAATTTTGGT |
| | | TGGGCCTAGAGAAGATATACTCCATAGTGAAGCAATCTAAT |
| | | TATGTTTTACGAATTGAGTTGGAAGACTGGAAAGACAACAA |
| | | ACATTATATTGAATATTCTTTTTACTTGGGAAATCACGAAA |
| | | CCAACTATACGCTACATCTAGTTGCGATTACTGGCAATGTC |
| | | CCCAATGCAATCCCGGAAAACAAAGATTTGGTGTTTTCTAC |
| | | TTGGGATCACAAAGCAAAAGGACACTTCAACTGTCCAGAGG |
| | | GTTATTCAGGAGGCTGGTGGTGGCATGATGAGTGTGGAGAA |
| | | AACAACCTAAATGGTAAATATAACAAACCAAGAGCAAAATC |
| | | TAAGCCAGAGAGGAGAAGAGGATTATCTTGGAAGTCTCAAA |
| | | ATGGAAGGTTATACTCTATAAAATCAACCAAAATGTTGATC |
| | | CATCCAACAGATTCAGAAAGCTTTGAA |
| NEG276 | | |
| HCDR1 (Kabat) | 7 | SSWMQ |
| HCDR2 (Kabat) | 8 | EIDPSDNYANYNQKFQG |
| HCDR3 (Kabat) | 9 | GSYFSNFFDY |
| HCDR1 (Chothia) | 10 | AYTFTSS |
| HCDR2 (Chothia) | 11 | DPSDNY |
| HCDR3 (Chothia) | 12 | GSYFSNFFDY |
| VH | 13 | QVQLVQSGAEVKKPGASVKVSCKASAYTFTSSWMQWVRQAP |
| | | GQGLEWMGEIDPSDNYANYNQKFQGRVTLTVDTSTSTAYME |
| | | LSSLRSEDTAVYYCASGSYFSNFFDYWGQGTLVTVSS |
| DNA Encoding VH | 14 | CAGGTGCAGCTGGTGCAGTCTGGCGCCGAAGTGAAGAAACC |
| | | AGGCGCCAGCGTGAAGGTGTCCTGCAAGGCCAGCGCCTACA |
| | | CCTTTACCAGCAGCTGGATGCAGTGGGTGCGCCAGGCTCCT |
| | | GGACAGGGCCTGGAATGGATGGGCGAGATCGACCCCAGCGA |
| | | CAACTACGCCAACTACAACCAGAAATTCCAGGGCAGAGTGA |
| | | CCCTGACCGTGGACACCAGCACCTCCACCGCCTACATGGAA |
| | | CTGAGCAGCCTGCGGAGCGAGGACACCGCCGTGTACTATTG |
| | | TGCCAGCGGCAGCTACTTCAGCAACTTCTTCGACTACTGGG |
| | | GCCAGGGCACCCTCGTGACCGTGTCATCT |
| Heavy Chain | 15 | QVQLVQSGAEVKKPGASVKVSCKASAYTFTSSWMQWVRQAP |
| | | GQGLEWMGEIDPSDNYANYNQKFQGRVTLTVDTSTSTAYME |
| | | LSSLRSEDTAVYYCASGSYFSNFFDYWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| DNA Encoding | 16 | CAGGTGCAGCTGGTGCAGTCTGGCGCCGAAGTGAAGAAACC |
| Heavy Chain | | AGGCGCCAGCGTGAAGGTGTCCTGCAAGGCCAGCGCCTACA |
| | | CCTTTACCAGCAGCTGGATGCAGTGGGTGCGCCAGGCTCCT |
| | | GGACAGGGCCTGGAATGGATGGGCGAGATCGACCCCAGCGA |
| | | CAACTACGCCAACTACAACCAGAAATTCCAGGGCAGAGTGA |
| | | CCCTGACCGTGGACACCAGCACCTCCACCGCCTACATGGAA |
| | | CTGAGCAGCCTGCGGAGCGAGGACACCGCCGTGTACTATTG |
| | | TGCCAGCGGCAGCTACTTCAGCAACTTCTTCGACTACTGGG |
| | | GCCAGGGCACCCTCGTGACCGTGTCATCTGCTAGCACCAAG |
| | | GGCCCCAGCGTGTTCCCCCTGGCCCCCAGCAGCAAGAGCAC |
| | | CAGCGGCGGCACAGCCGCCCTGGGCTGCCTGGTGAAGGACT |
| | | ACTTCCCCGAGCCCGTGACCGTGTCCTGGAACAGCGGAGCC |
| | | CTGACCTCCGGCGTGCACACCTTCCCCGCCGTGCTGCAGAG |
| | | CAGCGGCCTGTACAGCCTGTCCAGCGTGGTGACAGTGCCCA |
| | | GCAGCAGCCTGGGCACCCAGACCTACATCTGCAACGTGAAC |
| | | CACAAGCCCAGCAACACCAAGGTGGACAAGAGAGTGGAGCC |
| | | CAAGAGCTGCGACAAGACCCACACCTGCCCCCCCTGCCCAG |
| | | CCCCAGAGCTGCTGGGCGGACCCTCCGTGTTCCTGTTCCCC |
| | | CCCAAGCCCAAGGACACCCTGATGATCAGCAGGACCCCCGA |
| | | GGTGACCTGCGTGGTGGTGGACGTGAGCCACGAGGACCCAG |
| | | AGGTGAAGTTCAACTGGTACGTGGACGGCGTGGAGGTGCAC |
| | | AACGCCAAGACCAAGCCCAGAGAGGAGCAGTACAACAGCAC |
| | | CTACAGGGTGGTGTCCGTGCTGACCGTGCTGCACCAGGACT |
| | | GGCTGAACGGCAAGGAATACAAGTGCAAGGTCTCCAACAAG |
| | | GCCCTGCCAGCCCCCATCGAAAAGACCATCAGCAAGGCCAA |
| | | GGGCCAGCCACGGGAGCCCCAGGTGTACACCCTGCCCCCCT |
| | | CCCGGGAGGAGATGACCAAGAACCAGGTGTCCCTGACCTGT |
| | | CTGGTGAAGGGCTTCTACCCCAGCGACATCGCCGTGGAGTG |
| | | GGAGAGCAACGGCCAGCCCGAGAACAACTACAAGACCACCC |
| | | CCCCAGTGCTGGACAGCGACGGCAGCTTCTTCCTGTACAGC |
| | | AAGCTGACCGTGGACAAGTCCAGGTGGCAGCAGGGCAACGT |
| | | GTTCAGCTGCAGCGTGATGCACGAGGCCCTGCACAACCACT |
| | | ACACCCAGAAGAGCCTGAGCCTGTCCCCCGGCAAG |
| LCDR1 (Kabat) | 17 | KASQDIGSNLN |
| LCDR2 (Kabat) | 18 | AVSNRGP |
| LCDR3 (Kabat) | 19 | LQYASSPWT |
| LCDR1 (Chothia) | 20 | SQDIGSN |
| LCDR2 (Chothia) | 21 | AVS |
| LCDR3 (Chothia) | 32 | YASSPW |
| VL | 23 | EIVMTQSPATLSVSPGERATLSCKASQDIGSNLNWLQQKPG |
| | | QAPRRLIYAVSNRGPGIPARFSGSRSGSEYTLTISSLQSED |
| | | FAVYYCLQYASSPWTFGQGTKVEIK |
| DNA Encoding VL | 24 | GAGATCGTGATGACACAGAGCCCCGCCACCCTGTCCGTGTC |
| | | TCCAGGCGAAAGAGCCACCCTGAGCTGCAAAGCCAGCCAGG |
| | | ACATCGGCAGCAACCTGAACTGGCTGCAGCAGAAACCAGGC |
| | | CAGGCCCCCAGAAGGCTGATCTACGCTGTTTCCAACCGTGG |
| | | TCCTGGCATCCCCGCCAGATTTTCCGGCAGCAGATCCGGCA |
| | | GCGAGTACACCCTGACCATCAGCAGCCTGCAGAGCGAGGAC |
| | | TTCGCCGTGTACTACTGCCTGCAGTACGCCAGCAGCCCCTG |
| | | GACATTTGGCCAGGGCACCAAGGTGGAAATCAAG |
| Light Chain | 25 | EIVMTQSPATLSVSPGERATLSCKASQDIGSNLNWLQQKPG |
| | | QAPRRLIYAVSNRGPGIPARFSGSRSGSEYTLTISSLQSED |
| | | FAVYYCLQYASSPWTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| DNA encoding | 26 | GAGATCGTGATGACACAGAGCCCCGCCACCCTGTCCGTGTC |
| Light Chain | | TCCAGGCGAAAGAGCCACCCTGAGCTGCAAAGCCAGCCAGG |
| | | ACATCGGCAGCAACCTGAACTGGCTGCAGCAGAAACCAGGC |
| | | CAGGCCCCCAGAAGGCTGATCTACGCTGTTTCCAACCGTGG |
| | | TCCTGGCATCCCCGCCAGATTTTCCGGCAGCAGATCCGGCA |
| | | GCGAGTACACCCTGACCATCAGCAGCCTGCAGAGCGAGGAC |
| | | TTCGCCGTGTACTACTGCCTGCAGTACGCCAGCAGCCCCTG |
| | | GACATTTGGCCAGGGCACCAAGGTGGAAATCAAGCGTACGG |
| | | TGGCCGCTCCCAGCGTGTTCATCTTCCCCCCCAGCGACGAG |
| | | CAGCTGAAGAGCGGCACCGCCAGCGTGGTGTGCCTGCTGAA |
| | | CAACTTCTACCCCCGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAAGCATAAGGTGT |
| | | ACGCCTGCGAGGTGACCCACCAGGGCCTGTCCAGCCCCGTG |
| | | ACCAAGAGCTTCAACAGGGGCGAGTGC |
| NEG276-LALA | | |
| HCDR1 (Kabat) | 7 | SSWMQ |
| HCDR2 (Kabat) | 8 | EIDPSDNYANYNQKFQG |
| HCDR3 (Kabat) | 9 | GSYFSNFFDY |
| HCDR1 (Chothia) | 10 | AYTFTSS |
| HCDR2 (Chothia) | 11 | DPSDNY |
| HCDR3 (Chothia) | 12 | GSYFSNFFDY |
| VH | 13 | QVQLVQSGAEVKKPGASVKVSCKASAYTFTSSWMQWVRQAP |
| | | GQGLEWMGEIDPSDNYANYNQKFQGRVTLTVDTSTSTAYME |
| | | LSSLRSEDTAVYYCASGSYFSNFFDYWGQGTLVTVSS |
| DNA Encoding VH | 27 | CAGGTGCAGCTGGTGCAGTCAGGCGCCGAAGTGAAGAAACC |
| | | CGGCGCTAGTGTGAAAGTCAGCTGTAAAGCTAGTGCCTACA |
| | | CCTTCACCTCTAGCTGGATGCAGTGGGTCAGACAGGCCCCA |
| | | GGTCAGGGCCTGGAGTGGATGGGCGAGATCGACCCTAGCGA |
| | | TAACTACGCTAACTATAATCAGAAGTTTCAGGGTAGAGTCA |
| | | CCCTGACCGTGGACACTAGCACTAGCACCGCCTATATGGAA |
| | | CTGTCTAGCCTGAGATCAGAGGACACCGCCGTCTACTACTG |
| | | CGCTAGTGGTAGCTACTTCTCTAACTTCTTCGACTACTGGG |
| | | GTCAGGGCACCCTGGTCACCGTGTCTAGC |
| Heavy Chain | 28 | QVQLVQSGAEVKKPGASVKVSCKASAYTFTSSWMQWVRQAP |
| | | GQGLEWMGEIDPSDNYANYNQKFQGRVTLTVDTSTSTAYME |
| | | LSSLRSEDTAVYYCASGSYFSNFFDYWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPEAAGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| DNA Encoding | 29 | CAGGTGCAGCTGGTGCAGTCAGGCGCCGAAGTGAAGAAACC |
| Heavy Chain | | CGGCGCTAGTGTGAAAGTCAGCTGTAAAGCTAGTGCCTACA |
| | | CCTTCACCTCTAGCTGGATGCAGTGGGTCAGACAGGCCCCA |
| | | GGTCAGGGCCTGGAGTGGATGGGCGAGATCGACCCTAGCGA |
| | | TAACTACGCTAACTATAATCAGAAGTTTCAGGGTAGAGTCA |
| | | CCCTGACCGTGGACACTAGCACTAGCACCGCCTATATGGAA |
| | | CTGTCTAGCCTGAGATCAGAGGACACCGCCGTCTACTACTG |
| | | CGCTAGTGGTAGCTACTTCTCTAACTTCTTCGACTACTGGG |
| | | GTCAGGGCACCCTGGTCACCGTGTCTAGCGCTAGCACTAAG |
| | | GGCCCCTCCGTGTTCCCTCTGGCCCCTTCCAGCAAGTCTAC |
| | | CTCCGGCGGCACAGCTGCTCTGGGCTGCCTGGTCAAGGACT |
| | | ACTTCCCTGAGCCTGTGACAGTGTCCTGGAACTCTGGCGCC |
| | | CTGACCTCTGGCGTGCACACCTTCCCTGCCGTGCTGCAGTC |
| | | CTCCGGCCTGTACTCCCTGTCCTCCGTGGTCACAGTGCCTT |
| | | CAAGCAGCCTGGGCACCCAGACCTATATCTGCAACGTGAAC |
| | | CACAAGCCTTCCAACACCAAGGTGGACAAGCGGGTGGAGCC |
| | | TAAGTCCTGCGACAAGACCCACACCTGTCCTCCCTGCCCTG |
| | | CTCCTGAAGCTGCTGGCGGCCCTTCTGTGTTCCTGTTCCCT |
| | | CCAAAGCCCAAGGACACCCTGATGATCTCCCGGACCCCTGA |
| | | AGTGACCTGCGTGGTGGTGGACGTGTCCCACGAGGATCCTG |
| | | AAGTGAAGTTCAATTGGTACGTGGACGGCGTGGAGGTGCAC |
| | | AACGCCAAGACCAAGCCTCGGGAGGAACAGTACAACTCCAC |
| | | CTACCGGGTGGTGTCCGTGCTGACCGTGCTGCACCAGGACT |
| | | GGCTGAACGGCAAAGAGTACAAGTGCAAAGTCTCCAACAAG |
| | | GCCCTGCCTGCCCCTATCGAAAAGACAATCTCCAAGGCCAA |
| | | GGGCCAGCCTAGGGAACCCCAGGTGTACACCCTGCCACCCA |
| | | GCCGGGAGGAAATGACCAAGAACCAGGTGTCCCTGACCTGT |
| | | CTGGTCAAGGGCTTCTACCCTTCCGATATCGCCGTGGAGTG |
| | | GGAGTCTAACGGCCAGCCTGAGAACAACTACAAGACCACCC |
| | | CTCCTGTGCTGGACTCCGACGGCTCCTTCTTCCTGTACTCC |
| | | AAACTGACCGTGGACAAGTCCCGGTGGCAGCAGGGCAACGT |
| | | GTTCTCCTGCTCCGTGATGCACGAGGCCCTGCACAACCACT |
| | | ACACCCAGAAGTCCCTGTCCCTGTCTCCCGGCAAG |
| LCDR1 (Kabat) | 17 | KASQDIGSNLN |
| LCDR2 (Kabat) | 18 | AVSNRGP |
| LCDR3 (Kabat) | 19 | LQYASSPWT |
| LCDR1 (Chothia) | 20 | SQDIGSN |
| LCDR2 (Chothia) | 21 | AVS |
| LCDR3 (Chothia) | 22 | YASSPW |
| VL | 23 | EIVMTQSPATLSVSPGERATLSCKASQDIGSNLNWLQQKPG |
| | | QAPRRLIYAVSNRGPGIPARFSGSRSGSEYTLTISSLQSED |
| | | FAVYYCLQYASSPWTFGQGTKVEIK |
| DNA Encoding VL | 30 | GAGATCGTGATGACTCAGTCACCCGCTACCCTGAGCGTCAG |
| | | CCCTGGCGAGCGGGCTACACTGAGCTGTAAAGCCTCTCAGG |
| | | ATATCGGCTCTAACCTGAACTGGCTGCAGCAGAAGCCCGGT |
| | | CAGGCCCCTAGACGGCTGATCTACGCCGTGTCTAATAGAGG |
| | | CCCCGGAATCCCCGCTAGGTTTAGCGGCTCTAGGTCAGGTT |
| | | CAGAGTACACCCTGACTATCTCTAGCCTGCAGTCAGAGGAC |
| | | TTCGCCGTCTACTACTGCCTGCAGTACGCCTCTAGCCCCTG |
| | | GACCTTCGGTCAGGGCACTAAGGTCGAGATTAAG |
| Light Chain | 25 | EIVMTQSPATLSVSPGERATLSCKASQDIGSNLNWLQQKPG |
| | | QAPRRLIYAVSNRGPGIPARFSGSRSGSEYTLTISSLQSED |
| | | FAVYYCLQYASSPWTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| DNA Encoding | 31 | GAGATCGTGATGACTCAGTCACCCGCTACCCTGAGCGTCAG |
| Light Chain | | CCCTGGCGAGCGGGCTACACTGAGCTGTAAAGCCTCTCAGG |
| | | ATATCGGCTCTAACCTGAACTGGCTGCAGCAGAAGCCCGGT |
| | | CAGGCCCCTAGACGGCTGATCTACGCCGTGTCTAATAGAGG |
| | | CCCCGGAATCCCCGCTAGGTTTAGCGGCTCTAGGTCAGGTT |
| | | CAGAGTACACCCTGACTATCTCTAGCCTGCAGTCAGAGGAC |
| | | TTCGCCGTCTACTACTGCCTGCAGTACGCCTCTAGCCCCTG |
| | | GACCTTCGGTCAGGGCACTAAGGTCGAGATTAAGCGTACGG |
| | | TGGCCGCTCCCAGCGTGTTCATCTTCCCCCCCAGCGACGAG |
| | | CAGCTGAAGAGCGGCACCGCCAGCGTGGTGTGCCTGCTGAA |
| | | CAACTTCTACCCCCGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAAGCATAAGGTGT |
| | | ACGCCTGCGAGGTGACCCACCAGGGCCTGTCCAGCCCCGTG |
| | | ACCAAGAGCTTCAACAGGGGCGAGTGC |
| NEG278 | | |
| HCDR1 (Kabat) | 32 | SSWMQ |
| HCDR2 (Kabat) | 33 | EIDPSDNYANYNQKFQG |
| HCDR3 (Kabat) | 34 | GSYFSNFFDY |
| HCDR1 (Chothia) | 35 | AYTFTSS |
| HCDR2 (Chothia) | 36 | DPSDNY |
| HCDR3 (Chothia) | 37 | GSYFSNFFDY |
| VH | 38 | QVQLVQSGAEVKKPGASVKVSCKASAYTFTSSWMQWVRQAP |
| | | GQGLEWMGEIDPSDNYANYNQKFQGRVTLTVDTSTSTAYME |
| | | LSSLRSEDTAVYYCASGSYFSNFFDYWGQGTLVTVSS |
| DNA Encoding VH | 39 | CAGGTGCAGCTGGTGCAGTCTGGCGCCGAAGTGAAGAAACC |
| | | AGGCGCCAGCGTGAAGGTGTCCTGCAAGGCCAGCGCCTACA |
| | | CCTTTACCAGCAGCTGGATGCAGTGGGTGCGCCAGGCTCCT |
| | | GGACAGGGCCTGGAATGGATGGGCGAGATCGACCCCAGCGA |
| | | CAACTACGCCAACTACAACCAGAAATTCCAGGGCAGAGTGA |
| | | CCCTGACCGTGGACACCAGCACCTCCACCGCCTACATGGAA |
| | | CTGAGCAGCCTGCGGAGCGAGGACACCGCCGTGTACTATTG |
| | | TGCCAGCGGCAGCTACTTCAGCAACTTCTTCGACTACTGGG |
| | | GCCAGGGCACCCTCGTGACCGTGTCATCT |
| Heavy Chain | 40 | QVQLVQSGAEVKKPGASVKVSCKASAYTFTSSWMQWVRQAP |
| | | GQGLEWMGEIDPSDNYANYNQKFQGRVTLTVDTSTSTAYME |
| | | LSSLRSEDTAVYYCASGSYFSNFFDYWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| DNA Encoding | 41 | CAGGTGCAGCTGGTGCAGTCTGGCGCCGAAGTGAAGAAACC |
| Heavy Chain | | AGGCGCCAGCGTGAAGGTGTCCTGCAAGGCCAGCGCCTACA |
| | | CCTTTACCAGCAGCTGGATGCAGTGGGTGCGCCAGGCTCCT |
| | | GGACAGGGCCTGGAATGGATGGGCGAGATCGACCCCAGCGA |
| | | CAACTACGCCAACTACAACCAGAAATTCCAGGGCAGAGTGA |
| | | CCCTGACCGTGGACACCAGCACCTCCACCGCCTACATGGAA |
| | | CTGAGCAGCCTGCGGAGCGAGGACACCGCCGTGTACTATTG |
| | | TGCCAGCGGCAGCTACTTCAGCAACTTCTTCGACTACTGGG |
| | | GCCAGGGCACCCTCGTGACCGTGTCATCTGCTAGCACCAAG |
| | | GGCCCCAGCGTGTTCCCCCTGGCCCCCAGCAGCAAGAGCAC |
| | | CAGCGGCGGCACAGCCGCCCTGGGCTGCCTGGTGAAGGACT |
| | | ACTTCCCCGAGCCCGTGACCGTGTCCTGGAACAGCGGAGCC |
| | | CTGACCTCCGGCGTGCACACCTTCCCCGCCGTGCTGCAGAG |
| | | CAGCGGCCTGTACAGCCTGTCCAGCGTGGTGACAGTGCCCA |
| | | GCAGCAGCCTGGGCACCCAGACCTACATCTGCAACGTGAAC |
| | | CACAAGCCCAGCAACACCAAGGTGGACAAGAGAGTGGAGCC |
| | | CAAGAGCTGCGACAAGACCCACACCTGCCCCCCCTGCCCAG |
| | | CCCCAGAGCTGCTGGGCGGACCCTCCGTGTTCCTGTTCCCC |
| | | CCCAAGCCCAAGGACACCCTGATGATCAGCAGGACCCCCGA |
| | | GGTGACCTGCGTGGTGGTGGACGTGAGCCACGAGGACCCAG |
| | | AGGTGAAGTTCAACTGGTACGTGGACGGCGTGGAGGTGCAC |
| | | AACGCCAAGACCAAGCCCAGAGAGGAGCAGTACAACAGCAC |
| | | CTACAGGGTGGTGTCCGTGCTGACCGTGCTGCACCAGGACT |
| | | GGCTGAACGGCAAGGAATACAAGTGCAAGGTCTCCAACAAG |
| | | GCCCTGCCAGCCCCCATCGAAAAGACCATCAGCAAGGCCAA |
| | | GGGCCAGCCACGGGAGCCCCAGGTGTACACCCTGCCCCCCT |
| | | CCCGGGAGGAGATGACCAAGAACCAGGTGTCCCTGACCTGT |
| | | CTGGTGAAGGGCTTCTACCCCAGCGACATCGCCGTGGAGTG |
| | | GGAGAGCAACGGCCAGCCCGAGAACAACTACAAGACCACCC |
| | | CCCCAGTGCTGGACAGCGACGGCAGCTTCTTCCTGTACAGC |
| | | AAGCTGACCGTGGACAAGTCCAGGTGGCAGCAGGGCAACGT |
| | | GTTCAGCTGCAGCGTGATGCACGAGGCCCTGCACAACCACT |
| | | ACACCCAGAAGAGCCTGAGCCTGTCCCCCGGCAAG |
| LCDR1 (Kabat) | 42 | KASQDIGSNLN |
| LCDR2 (Kabat) | 43 | AASVREP |
| LCDR3 (Kabat) | 44 | LQYASSPWT |
| LCDR1 (Chothia) | 45 | SQDIGSN |
| LCDR2 (Chothia) | 46 | AAS |
| LCDR3 (Chothia) | 47 | YASSPW |
| VL | 48 | EIVMTQSPATLSVSPGERATLSCKASQDIGSNLNWLQQKPG |
| | | QAPRRLIYAASVREPGIPARFSGSRSGSEYTLTISSLQSED |
| | | FAVYYCLQYASSPWTFGQGTKVEIK |
| DNA Encoding VL | 49 | GAGATCGTGATGACACAGAGCCCCGCCACCCTGTCCGTGTC |
| | | TCCAGGCGAAAGAGCCACCCTGAGCTGCAAAGCCAGCCAGG |
| | | ACATCGGCAGCAACCTGAACTGGCTGCAGCAGAAACCAGGC |
| | | CAGGCCCCCAGAAGGCTGATCTACGCTGCTTCCGTCCGTGA |
| | | GCCTGGCATCCCCGCCAGATTTTCCGGCAGCAGATCCGGCA |
| | | GCGAGTACACCCTGACCATCAGCAGCCTGCAGAGCGAGGAC |
| | | TTCGCCGTGTACTACTGCCTGCAGTACGCCAGCAGCCCCTG |
| | | GACATTTGGCCAGGGCACCAAGGTGGAAATCAAG |
| Light Chain | 50 | EIVMTQSPATLSVSPGERATLSCKASQDIGSNLNWLQQKPG |
| | | QAPRRLIYAASVREPGIPARFSGSRSGSEYTLTISSLQSED |
| | | FAVYYCLQYASSPWTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| DNA Encoding | 51 | GAGATCGTGATGACACAGAGCCCCGCCACCCTGTCCGTGTC |
| Light Chain | | TCCAGGCGAAAGAGCCACCCTGAGCTGCAAAGCCAGCCAGG |
| | | ACATCGGCAGCAACCTGAACTGGCTGCAGCAGAAACCAGGC |
| | | CAGGCCCCCAGAAGGCTGATCTACGCTGCTTCCGTCCGTGA |
| | | GCCTGGCATCCCCGCCAGATTTTCCGGCAGCAGATCCGGCA |
| | | GCGAGTACACCCTGACCATCAGCAGCCTGCAGAGCGAGGAC |
| | | TTCGCCGTGTACTACTGCCTGCAGTACGCCAGCAGCCCCTG |
| | | GACATTTGGCCAGGGCACCAAGGTGGAAATCAAGCGTACGG |
| | | TGGCCGCTCCCAGCGTGTTCATCTTCCCCCCCAGCGACGAG |
| | | CAGCTGAAGAGCGGCACCGCCAGCGTGGTGTGCCTGCTGAA |
| | | CAACTTCTACCCCCGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAAGCATAAGGTGT |
| | | ACGCCTGCGAGGTGACCCACCAGGGCCTGTCCAGCCCCGTG |
| | | ACCAAGAGCTTCAACAGGGGCGAGTGC |
| NEG310 | | |
| HCDR1 (Kabat) | 52 | SYTMH |
| HCDR2 (Kabat) | 53 | YINPSSGYTKYNQKFQG |
| HCDR3 (Kabat) | 54 | GWLLLAMDY |
| HCDR1 (Chothia) | 55 | GYTFTSY |
| HCDR2 (Chothia) | 56 | NPSSGY |
| HCDR3 (Chothia) | 57 | GWLLLAMDY |
| VH | 58 | QVQLVQSGAEVKKPGASVKVSCKASGYTFTSYTMHWVRQAP |
| | | GQGLEWMGYINPSSGYTKYNQKFQGRVTMTADKSTSTAYME |
| | | LSSLRSEDTAVYYCAEGWLLLAMDYWGQGTLVTVSS |
| DNA Encoding VH | 59 | CAGGTGCAGCTGGTGCAGTCTGGCGCCGAAGTGAAGAAACC |
| | | AGGCGCCAGCGTGAAGGTGTCCTGCAAGGCCAGCGGCTACA |
| | | CCTTTACCAGCTACACCATGCACTGGGTGCGCCAGGCTCCA |
| | | GGCCAGGGACTGGAATGGATGGGCTACATCAACCCCAGCAG |
| | | CGGCTATACCAAGTACAACCAGAAATTCCAGGGCCGCGTGA |
| | | CCATGACCGCCGACAAGAGCACAAGCACCGCCTACATGGAA |
| | | CTGAGCAGCCTGCGGAGCGAGGACACCGCCGTGTACTATTG |
| | | TGCCGAGGGCTGGCTGCTGCTGGCCATGGATTATTGGGGCC |
| | | AGGGCACCCTCGTGACCGTGTCTAGT |
| Heavy Chain | 60 | QVQLVQSGAEVKKPGASVKVSCKASGYTFTSYTMHWVRQAP |
| | | GQGLEWMGYINPSSGYTKYNQKFQGRVTMTADKSTSTAYME |
| | | LSSLRSEDTAVYYCAEGWLLLAMDYWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKRVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSREEMTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGK |
| DNA Encoding | 61 | CAGGTGCAGCTGGTGCAGTCTGGCGCCGAAGTGAAGAAACC |
| Heavy Chain | | AGGCGCCAGCGTGAAGGTGTCCTGCAAGGCCAGCGGCTACA |
| | | CCTTTACCAGCTACACCATGCACTGGGTGCGCCAGGCTCCA |
| | | GGCCAGGGACTGGAATGGATGGGCTACATCAACCCCAGCAG |
| | | CGGCTATACCAAGTACAACCAGAAATTCCAGGGCCGCGTGA |
| | | CCATGACCGCCGACAAGAGCACAAGCACCGCCTACATGGAA |
| | | CTGAGCAGCCTGCGGAGCGAGGACACCGCCGTGTACTATTG |
| | | TGCCGAGGGCTGGCTGCTGCTGGCCATGGATTATTGGGGCC |
| | | AGGGCACCCTCGTGACCGTGTCTAGTGCTAGCACCAAGGGC |
| | | CCCAGCGTGTTCCCCCTGGCCCCCAGCAGCAAGAGCACCAG |
| | | CGGCGGCACAGCCGCCCTGGGCTGCCTGGTGAAGGACTACT |
| | | TCCCCGAGCCCGTGACCGTGTCCTGGAACAGCGGAGCCCTG |
| | | ACCTCCGGCGTGCACACCTTCCCCGCCGTGCTGCAGAGCAG |
| | | CGGCCTGTACAGCCTGTCCAGCGTGGTGACAGTGCCCAGCA |
| | | GCAGCCTGGGCACCCAGACCTACATCTGCAACGTGAACCAC |
| | | AAGCCCAGCAACACCAAGGTGGACAAGAGAGTGGAGCCCAA |
| | | GAGCTGCGACAAGACCCACACCTGCCCCCCCTGCCCAGCCC |
| | | CAGAGCTGCTGGGCGGACCCTCCGTGTTCCTGTTCCCCCCC |
| | | AAGCCCAAGGACACCCTGATGATCAGCAGGACCCCCGAGGT |
| | | GACCTGCGTGGTGGTGGACGTGAGCCACGAGGACCCAGAGG |
| | | TGAAGTTCAACTGGTACGTGGACGGCGTGGAGGTGCACAAC |
| | | GCCAAGACCAAGCCCAGAGAGGAGCAGTACAACAGCACCTA |
| | | CAGGGTGGTGTCCGTGCTGACCGTGCTGCACCAGGACTGGC |
| | | TGAACGGCAAGGAATACAAGTGCAAGGTCTCCAACAAGGCC |
| | | CTGCCAGCCCCCATCGAAAAGACCATCAGCAAGGCCAAGGG |
| | | CCAGCCACGGGAGCCCCAGGTGTACACCCTGCCCCCCTCCC |
| | | GGGAGGAGATGACCAAGAACCAGGTGTCCCTGACCTGTCTG |
| | | GTGAAGGGCTTCTACCCCAGCGACATCGCCGTGGAGTGGGA |
| | | GAGCAACGGCCAGCCCGAGAACAACTACAAGACCACCCCCC |
| | | CAGTGCTGGACAGCGACGGCAGCTTCTTCCTGTACAGCAAG |
| | | CTGACCGTGGACAAGTCCAGGTGGCAGCAGGGCAACGTGTT |
| | | CAGCTGCAGCGTGATGCACGAGGCCCTGCACAACCACTACA |
| | | CCCAGAAGAGCCTGAGCCTGTCCCCCGGCAAG |
| LCDR1 (Kabat) | 62 | RSSTGAVTTSNYAI |
| LCDR2 (Kabat) | 63 | GTNNRAP |
| LCDR3 (Kabat) | 64 | ALWYSDHWV |
| LCDR1 (Chothia) | 65 | STGAVTTSNY |
| LCDR2 (Chothia) | 66 | GTN |
| LCDR3 (Chothia) | 67 | WYSDHW |
| VL | 68 | EAVVTQSPATLSLSPGERATLSCRSSTGAVTTSNYAIWVQE |
| | | KPGQAPRGLIGGTNNRAPGIPARFSGSLSGDDATLTISSLQ |
| | | PEDFAVYFCALWYSDHWVFGQGTKVEIK |
| DNA Encoding VL | 69 | GAAGCCGTCGTGACACAGAGCCCTGCCACCCTGTCACTGAG |
| | | CCCTGGCGAAAGAGCCACCCTGAGCTGCAGATCTAGCACCG |
| | | GCGCTGTGACCACCAGCAACTACGCCATCTGGGTGCAGGAA |
| | | AAGCCCGGCCAGGCTCCCAGAGGACTGATCGGCGGCACCAA |
| | | CAATAGAGCCCCTGGCATCCCCGCCAGATTCAGCGGATCTC |
| | | TGTCTGGCGACGACGCCACACTGACCATCAGCAGCCTGCAG |
| | | CCCGAGGACTTCGCCGTGTACTTCTGCGCCCTGTGGTACAG |
| | | CGACCACTGGGTGTTCGGCCAGGGCACCAAGGTGGAAATCA |
| | | AG |
| Light Chain | 70 | EAVVTQSPATLSLSPGERATLSCRSSTGAVTTSNYAIWVQE |
| | | KPGQAPRGLIGGTNNRAPGIPARFSGSLSGDDATLTISSLQ |
| | | PEDFAVYFCALWYSDHWVFGQGTKVEIKRTVAAPSVFIFPP |
| | | SDEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| DNA Encoding | 71 | GAAGCCGTCGTGACACAGAGCCCTGCCACCCTGTCACTGAG |
| Light Chain | | CCCTGGCGAAAGAGCCACCCTGAGCTGCAGATCTAGCACCG |
| | | GCGCTGTGACCACCAGCAACTACGCCATCTGGGTGCAGGAA |
| | | AAGCCCGGCCAGGCTCCCAGAGGACTGATCGGCGGCACCAA |
| | | CAATAGAGCCCCTGGCATCCCCGCCAGATTCAGCGGATCTC |
| | | TGTCTGGCGACGACGCCACACTGACCATCAGCAGCCTGCAG |
| | | CCCGAGGACTTCGCCGTGTACTTCTGCGCCCTGTGGTACAG |
| | | CGACCACTGGGTGTTCGGCCAGGGCACCAAGGTGGAAATCA |
| | | AGCGTACGGTGGCCGCTCCCAGCGTGTTCATCTTCCCCCCC |
| | | AGCGACGAGCAGCTGAAGAGCGGCACCGCCAGCGTGGTGTG |
| | | CCTGCTGAACAACTTCTACCCCCGGGAGGCCAAGGTGCAGT |
| | | GGAAGGTGGACAACGCCCTGCAGAGCGGCAACAGCCAGGAG |
| | | AGCGTCACCGAGCAGGACAGCAAGGACTCCACCTACAGCCT |
| | | GAGCAGCACCCTGACCCTGAGCAAGGCCGACTACGAGAAGC |
| | | ATAAGGTGTACGCCTGCGAGGTGACCCACCAGGGCCTGTCC |
| | | AGCCCCGTGACCAAGAGCTTCAACAGGGGCGAGTGC |
| NEG313 | | |
| HCDR1 (Kabat) | 72 | NYWIT |
| HCDR2 (Kabat) | 73 | DFYPGGGSTNYNAKLQG |
| HCDR3 (Kabat) | 74 | SPPQVAPFDY |
| HCDR1 (Chothia) | 75 | GYTFNNY |
| HCDR2 (Chothia) | 76 | YPGGGS |
| HCDR3 (Chothia) | 77 | SPPQVAPFDY |
| VH | 78 | QVQLVQSGAEVKKPGASVKVSCKASGYTFNNYWITWVRQAP |
| | | GQGLEWMGDFYPGGGSTNYNAKLQGRVTLTVDTSTSTAYME |
| | | LRSLRSDDTAVYYCARSPPQVAPFDYWGQGTLVTVSS |
| DNA encoding VH | 79 | CAGGTGCAGCTGGTGCAGTCTGGCGCCGAAGTGAAGAAACC |
| | | AGGCGCCAGCGTGAAGGTGTCCTGCAAGGCCAGCGGCTACA |
| | | CCTTTAACAACTACTGGATCACCTGGGTGCGCCAGGCCCCT |
| | | GGACAGGGACTGGAATGGATGGGCGACTTCTACCCTGGCGG |
| | | CGGCAGCACCAACTACAACGCCAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTATTACTG |
| | | CGCTAGAAGCCCTCCTCAGGTGGCCCCCTTCGATTATTGGG |
| | | GCCAGGGCACACTCGTGACCGTGTCCTCT |
| Heavy Chain | 80 | QVQLVQSGAEVKKPGASVKVSCKASGYTFNNYWITWVRQAP |
| | | GQGLEWMGDFYPGGGSTNYNAKLQGRVTLTVDTSTSTAYME |
| | | LRSLRSDDTAVYYCARSPPQVAPFDYWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| DNA Encoding | 81 | CAGGTGCAGCTGGTGCAGTCTGGCGCCGAAGTGAAGAAACC |
| Chain | | AGGCGCCAGCGTGAAGGTGTCCTGCAAGGCCAGCGGCTACA |
| | | CCTTTAACAACTACTGGATCACCTGGGTGCGCCAGGCCCCT |
| | | GGACAGGGACTGGAATGGATGGGCGACTTCTACCCTGGCGG |
| | | CGGCAGCACCAACTACAACGCCAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTATTACTG |
| | | CGCTAGAAGCCCTCCTCAGGTGGCCCCCTTCGATTATTGGG |
| | | GCCAGGGCACACTCGTGACCGTGTCCTCTGCTAGCACCAAG |
| | | GGCCCCAGCGTGTTCCCCCTGGCCCCCAGCAGCAAGAGCAC |
| | | CAGCGGCGGCACAGCCGCCCTGGGCTGCCTGGTGAAGGACT |
| | | ACTTCCCCGAGCCCGTGACCGTGTCCTGGAACAGCGGAGCC |
| | | CTGACCTCCGGCGTGCACACCTTCCCCGCCGTGCTGCAGAG |
| | | CAGCGGCCTGTACAGCCTGTCCAGCGTGGTGACAGTGCCCA |
| | | GCAGCAGCCTGGGCACCCAGACCTACATCTGCAACGTGAAC |
| | | CACAAGCCCAGCAACACCAAGGTGGACAAGAGAGTGGAGCC |
| | | CAAGAGCTGCGACAAGACCCACACCTGCCCCCCCTGCCCAG |
| | | CCCCAGAGCTGCTGGGCGGACCCTCCGTGTTCCTGTTCCCC |
| | | CCCAAGCCCAAGGACACCCTGATGATCAGCAGGACCCCCGA |
| | | GGTGACCTGCGTGGTGGTGGACGTGAGCCACGAGGACCCAG |
| | | AGGTGAAGTTCAACTGGTACGTGGACGGCGTGGAGGTGCAC |
| | | AACGCCAAGACCAAGCCCAGAGAGGAGCAGTACAACAGCAC |
| | | CTACAGGGTGGTGTCCGTGCTGACCGTGCTGCACCAGGACT |
| | | GGCTGAACGGCAAGGAATACAAGTGCAAGGTCTCCAACAAG |
| | | GCCCTGCCAGCCCCCATCGAAAAGACCATCAGCAAGGCCAA |
| | | GGGCCAGCCACGGGAGCCCCAGGTGTACACCCTGCCCCCCT |
| | | CCCGGGAGGAGATGACCAAGAACCAGGTGTCCCTGACCTGT |
| | | CTGGTGAAGGGCTTCTACCCCAGCGACATCGCCGTGGAGTG |
| | | GGAGAGCAACGGCCAGCCCGAGAACAACTACAAGACCACCC |
| | | CCCCAGTGCTGGACAGCGACGGCAGCTTCTTCCTGTACAGC |
| | | AAGCTGACCGTGGACAAGTCCAGGTGGCAGCAGGGCAACGT |
| | | GTTCAGCTGCAGCGTGATGCACGAGGCCCTGCACAACCACT |
| | | ACACCCAGAAGAGCCTGAGCCTGTCCCCCGGCAAG |
| LCDR1 (Kabat) | 82 | QASDYIYHWLG |
| LCDR2 (Kabat) | 83 | GASGLET |
| LCDR3 (Kabat) | 84 | QQYWSTPWT |
| LCDR1 (Chothia) | 85 | SDYIYHW |
| LCDR2 (Chothia) | 86 | GAS |
| LCDR3 (Chothia) | 87 | YWSTPW |
| VL | 88 | DIQMTQSPSSLSASVGDRVTITCQASDYIYHWLGWYQQKPG |
| | | KAPKLLISGASGLETGVPSRFSGSGSGKDYTFTISSLQPED |
| | | IATYYCQQYWSTPWTFGQGTKLEIK |
| DNA Encoding VL | 89 | GACATCCAGATGACCCAGAGCCCCAGCAGCCTGTCTGCCAG |
| | | CGTGGGCGACAGGGTGACCATCACCTGTCAGGCCAGCGACT |
| | | ACATCTACCACTGGCTGGGCTGGTATCAGCAGAAGCCCGGC |
| | | AAGGCCCCCAAGCTGCTGATTAGCGGAGCCTCCGGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCACCCCCTG |
| | | GACCTTTGGCCAGGGCACCAAGCTGGAAATCAAG |
| Light Chain | 90 | DIQMTQSPSSLSASVGDRVTITCQASDYIYHWLGWYQQKPG |
| | | KAPKLLISGASGLETGVPSRFSGSGSGKDYTFTISSLQPED |
| | | IATYYCQQYWSTPWTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| DNA Encoding | 91 | GACATCCAGATGACCCAGAGCCCCAGCAGCCTGTCTGCCAG |
| Light Chain | | CGTGGGCGACAGGGTGACCATCACCTGTCAGGCCAGCGACT |
| | | ACATCTACCACTGGCTGGGCTGGTATCAGCAGAAGCCCGGC |
| | | AAGGCCCCCAAGCTGCTGATTAGCGGAGCCTCCGGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCACCCCCTG |
| | | GACCTTTGGCCAGGGCACCAAGCTGGAAATCAAGCGTACGG |
| | | TGGCCGCTCCCAGCGTGTTCATCTTCCCCCCCAGCGACGAG |
| | | CAGCTGAAGAGCGGCACCGCCAGCGTGGTGTGCCTGCTGAA |
| | | CAACTTCTACCCCCGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAAGCATAAGGTGT |
| | | ACGCCTGCGAGGTGACCCACCAGGGCCTGTCCAGCCCCGTG |
| | | ACCAAGAGCTTCAACAGGGGCGAGTGC |
| NEG315 | | |
| HCDR1 (Kabat) | 92 | NYWIT |
| HCDR2 (Kabat) | 93 | DFYPGGGNTNYNAKLQG |
| HCDR3 (Kabat) | 94 | SPPQVAPFDY |
| HCDR1 (Chothia) | 95 | GYTFTNY |
| HCDR2 (Chothia) | 96 | YPGGGN |
| HCDR3 (Chothia) | 97 | SPPQVAPFDY |
| VH | 98 | QVQLVQSGAEVKKPGASVKVSCKASGYTFTNYWITWVRQAP |
| | | GQGLEWMGDFYPGGGNTNYNAKLQGRVTLTVDTSTSTAYME |
| | | LRSLRSDDTAVYYCARSPPQVAPFDYWGQGTLVTVSS |
| DNA Encoding VH | 99 | CAGGTGCAGCTGGTGCAGTCTGGCGCCGAAGTGAAGAAACC |
| | | AGGCGCCAGCGTGAAGGTGTCCTGCAAGGCCAGCGGCTACA |
| | | CCTTTACCAACTACTGGATCACCTGGGTGCGCCAGGCCCCT |
| | | GGACAGGGACTGGAATGGATGGGCGACTTCTACCCTGGCGG |
| | | CGGCAACACCAACTACAACGCCAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTATTACTG |
| | | CGCTAGAAGCCCTCCTCAGGTGGCCCCCTTCGATTATTGGG |
| | | GCCAGGGCACACTCGTGACCGTGTCCTCT |
| Heavy Chain | 100 | QVQLVQSGAEVKKPGASVKVSCKASGYTFTNYWITWVRQAP |
| | | GQGLEWMGDFYPGGGNTNYNAKLQGRVTLTVDTSTSTAYME |
| | | LRSLRSDDTAVYYCARSPPQVAPFDYWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| DNA Encoding | 101 | CAGGTGCAGCTGGTGCAGTCTGGCGCCGAAGTGAAGAAACC |
| Heavy Chain | | AGGCGCCAGCGTGAAGGTGTCCTGCAAGGCCAGCGGCTACA |
| | | CCTTTACCAACTACTGGATCACCTGGGTGCGCCAGGCCCCT |
| | | GGACAGGGACTGGAATGGATGGGCGACTTCTACCCTGGCGG |
| | | CGGCAACACCAACTACAACGCCAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTATTACTG |
| | | CGCTAGAAGCCCTCCTCAGGTGGCCCCCTTCGATTATTGGG |
| | | GCCAGGGCACACTCGTGACCGTGTCCTCTGCTAGCACCAAG |
| | | GGCCCCAGCGTGTTCCCCCTGGCCCCCAGCAGCAAGAGCAC |
| | | CAGCGGCGGCACAGCCGCCCTGGGCTGCCTGGTGAAGGACT |
| | | ACTTCCCCGAGCCCGTGACCGTGTCCTGGAACAGCGGAGCC |
| | | CTGACCTCCGGCGTGCACACCTTCCCCGCCGTGCTGCAGAG |
| | | CAGCGGCCTGTACAGCCTGTCCAGCGTGGTGACAGTGCCCA |
| | | GCAGCAGCCTGGGCACCCAGACCTACATCTGCAACGTGAAC |
| | | CACAAGCCCAGCAACACCAAGGTGGACAAGAGAGTGGAGCC |
| | | CAAGAGCTGCGACAAGACCCACACCTGCCCCCCCTGCCCAG |
| | | CCCCAGAGCTGCTGGGCGGACCCTCCGTGTTCCTGTTCCCC |
| | | CCCAAGCCCAAGGACACCCTGATGATCAGCAGGACCCCCGA |
| | | GGTGACCTGCGTGGTGGTGGACGTGAGCCACGAGGACCCAG |
| | | AGGTGAAGTTCAACTGGTACGTGGACGGCGTGGAGGTGCAC |
| | | AACGCCAAGACCAAGCCCAGAGAGGAGCAGTACAACAGCAC |
| | | CTACAGGGTGGTGTCCGTGCTGACCGTGCTGCACCAGGACT |
| | | GGCTGAACGGCAAGGAATACAAGTGCAAGGTCTCCAACAAG |
| | | GCCCTGCCAGCCCCCATCGAAAAGACCATCAGCAAGGCCAA |
| | | GGGCCAGCCACGGGAGCCCCAGGTGTACACCCTGCCCCCCT |
| | | CCCGGGAGGAGATGACCAAGAACCAGGTGTCCCTGACCTGT |
| | | CTGGTGAAGGGCTTCTACCCCAGCGACATCGCCGTGGAGTG |
| | | GGAGAGCAACGGCCAGCCCGAGAACAACTACAAGACCACCC |
| | | CCCCAGTGCTGGACAGCGACGGCAGCTTCTTCCTGTACAGC |
| | | AAGCTGACCGTGGACAAGTCCAGGTGGCAGCAGGGCAACGT |
| | | GTTCAGCTGCAGCGTGATGCACGAGGCCCTGCACAACCACT |
| | | ACACCCAGAAGAGCCTGAGCCTGTCCCCCGGCAAG |
| LCDR1 (Kabat) | 102 | QASEYIYNWLG |
| LCDR2 (Kabat) | 103 | GASGLET |
| LCDR3 (Kabat) | 104 | QQYWSTPWT |
| LCDR1 (Chothia) | 105 | SEYIYNW |
| LCDR2 (Chothia) | 106 | GAS |
| LCDR3 (Chothia) | 107 | YWSTPW |
| VL | 108 | DIQMTQSPSSLSASVGDRVTITCQASEYIYNWLGWYQQKPG |
| | | KAPKLLISGASGLETGVPSRFSGSGSGKDYTFTISSLQPED |
| | | IATYYCQQYWSTPWTFGQGTKLEIK |
| DNA Encoding VL | 109 | GACATCCAGATGACCCAGAGCCCCAGCAGCCTGTCTGCCAG |
| | | CGTGGGCGACAGGGTGACCATCACCTGTCAGGCCAGCGAAT |
| | | ACATCTACAACTGGCTGGGCTGGTATCAGCAGAAGCCCGGC |
| | | AAGGCCCCCAAGCTGCTGATTAGCGGAGCCTCCGGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCACCCCCTG |
| | | GACCTTTGGCCAGGGCACCAAGCTGGAAATCAAG |
| Light Chain | 110 | DIQMTQSPSSLSASVGDRVTITCQASEYIYNWLGWYQQKPG |
| | | KAPKLLISGASGLETGVPSRFSGSGSGKDYTFTISSLQPED |
| | | IATYYCQQYWSTPWTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| DNA Encoding | 111 | GACATCCAGATGACCCAGAGCCCCAGCAGCCTGTCTGCCAG |
| Light Chain | | CGTGGGCGACAGGGTGACCATCACCTGTCAGGCCAGCGAAT |
| | | ACATCTACAACTGGCTGGGCTGGTATCAGCAGAAGCCCGGC |
| | | AAGGCCCCCAAGCTGCTGATTAGCGGAGCCTCCGGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCACCCCCTG |
| | | GACCTTTGGCCAGGGCACCAAGCTGGAAATCAAGCGTACGG |
| | | TGGCCGCTCCCAGCGTGTTCATCTTCCCCCCCAGCGACGAG |
| | | CAGCTGAAGAGCGGCACCGCCAGCGTGGTGTGCCTGCTGAA |
| | | CAACTTCTACCCCCGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAAGCATAAGGTGT |
| | | ACGCCTGCGAGGTGACCCACCAGGGCCTGTCCAGCCCCGTG |
| | | ACCAAGAGCTTCAACAGGGGCGAGTGC |
| NEG318 | | |
| HCDR1 (Kabat) | 112 | SFWIT |
| HCDR2 (Kabat) | 113 | DIYPGGATTNYNEKLQG |
| HCDR3 (Kabat) | 114 | SPPQVGPFDY |
| HCDR1 (Chothia) | 115 | GYTFTSF |
| HCDR2 (Chothia) | 116 | YPGGAT |
| HCDR3 (Chothia) | 117 | SPPQVGPFDY |
| VH | 118 | QVQLVQSGAEVKKPGASVKVSCKASGYTFTSFWITWVRQAP |
| | | GQGLEWMGDIYPGGATTNYNEKLQGRVTLTVDTSTSTAYME |
| | | LRSLRSDDTAVYYCARSPPQVGPFDYWGQGTLVTVSS |
| DNA Encoding VH | 119 | CAGGTGCAGCTGGTGCAGTCTGGCGCCGAAGTGAAGAAACC |
| | | AGGCGCCAGCGTGAAGGTGTCCTGCAAGGCCAGCGGCTATA |
| | | CCTTCACCAGCTTTTGGATCACCTGGGTGCGCCAGGCCCCT |
| | | GGACAGGGACTGGAATGGATGGGCGACATCTACCCTGGCGG |
| | | CGCCACCACCAACTACAACGAGAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTACTACTG |
| | | CGCTAGAAGCCCTCCTCAGGTGGGCCCCTTCGATTATTGGG |
| | | GCCAGGGCACACTCGTGACCGTGTCCTCT |
| Heavy Chain | 120 | QVQLVQSGAEVKKPGASVKVSCKASGYTFTSFWITWVRQAP |
| | | GQGLEWMGDIYPGGATTNYNEKLQGRVTLTVDTSTSTAYME |
| | | LRSLRSDDTAVYYCARSPPQVGPFDYWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| DNA Encoding | 121 | CAGGTGCAGCTGGTGCAGTCTGGCGCCGAAGTGAAGAAACC |
| Heavy Chain | | AGGCGCCAGCGTGAAGGTGTCCTGCAAGGCCAGCGGCTATA |
| | | CCTTCACCAGCTTTTGGATCACCTGGGTGCGCCAGGCCCCT |
| | | GGACAGGGACTGGAATGGATGGGCGACATCTACCCTGGCGG |
| | | CGCCACCACCAACTACAACGAGAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTACTACTG |
| | | CGCTAGAAGCCCTCCTCAGGTGGGCCCCTTCGATTATTGGG |
| | | GCCAGGGCACACTCGTGACCGTGTCCTCTGCTAGCACCAAG |
| | | GGCCCCAGCGTGTTCCCCCTGGCCCCCAGCAGCAAGAGCAC |
| | | CAGCGGCGGCACAGCCGCCCTGGGCTGCCTGGTGAAGGACT |
| | | ACTTCCCCGAGCCCGTGACCGTGTCCTGGAACAGCGGAGCC |
| | | CTGACCTCCGGCGTGCACACCTTCCCCGCCGTGCTGCAGAG |
| | | CAGCGGCCTGTACAGCCTGTCCAGCGTGGTGACAGTGCCCA |
| | | GCAGCAGCCTGGGCACCCAGACCTACATCTGCAACGTGAAC |
| | | CACAAGCCCAGCAACACCAAGGTGGACAAGAGAGTGGAGCC |
| | | CAAGAGCTGCGACAAGACCCACACCTGCCCCCCCTGCCCAG |
| | | CCCCAGAGCTGCTGGGCGGACCCTCCGTGTTCCTGTTCCCC |
| | | CCCAAGCCCAAGGACACCCTGATGATCAGCAGGACCCCCGA |
| | | GGTGACCTGCGTGGTGGTGGACGTGAGCCACGAGGACCCAG |
| | | AGGTGAAGTTCAACTGGTACGTGGACGGCGTGGAGGTGCAC |
| | | AACGCCAAGACCAAGCCCAGAGAGGAGCAGTACAACAGCAC |
| | | CTACAGGGTGGTGTCCGTGCTGACCGTGCTGCACCAGGACT |
| | | GGCTGAACGGCAAGGAATACAAGTGCAAGGTCTCCAACAAG |
| | | GCCCTGCCAGCCCCCATCGAAAAGACCATCAGCAAGGCCAA |
| | | GGGCCAGCCACGGGAGCCCCAGGTGTACACCCTGCCCCCCT |
| | | CCCGGGAGGAGATGACCAAGAACCAGGTGTCCCTGACCTGT |
| | | CTGGTGAAGGGCTTCTACCCCAGCGACATCGCCGTGGAGTG |
| | | GGAGAGCAACGGCCAGCCCGAGAACAACTACAAGACCACCC |
| | | CCCCAGTGCTGGACAGCGACGGCAGCTTCTTCCTGTACAGC |
| | | AAGCTGACCGTGGACAAGTCCAGGTGGCAGCAGGGCAACGT |
| | | GTTCAGCTGCAGCGTGATGCACGAGGCCCTGCACAACCACT |
| | | ACACCCAGAAGAGCCTGAGCCTGTCCCCCGGCAAG |
| LCDR1 (Kabat) | 122 | QASDYIYHWLA |
| LCDR2 (Kabat) | 123 | GASSLET |
| LCDR3 (Kabat) | 124 | QQYWSIPWT |
| LCDR1 (Chothia) | 125 | SDYIYHW |
| LCDR2 (Chothia) | 126 | GAS |
| LCDR3 (Chothia) | 127 | YWSIPW |
| VL | 128 | DIQMTQSPSSLSASVGDRVTITCQASDYIYHWLAWYQQKPG |
| | | KAPKLLISGASSLETGVPSRFSGSGSGKDYTFTISSLQPED |
| | | IATYYCQQYWSIPWTFGQGTKLEIK |
| DNA Encoding VL | 129 | GACATCCAGATGACCCAGAGCCCCAGCAGCCTGTCTGCCAG |
| | | CGTGGGCGACAGAGTGACCATCACCTGTCAGGCCAGCGACT |
| | | ACATCTACCACTGGCTGGCCTGGTATCAGCAGAAGCCCGGC |
| | | AAGGCCCCCAAGCTGCTGATTAGCGGAGCCTCCAGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCATCCCCTG |
| | | GACCTTTGGCCAGGGCACCAAGCTGGAAATCAAG |
| Light Chain | 130 | DIQMTQSPSSLSASVGDRVTITCQASDYIYHWLAWYQQKPG |
| | | KAPKLLISGASSLETGVPSRFSGSGSGKDYTFTISSLQPED |
| | | IATYYCQQYWSIPWTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| DNA Encoding | 131 | GACATCCAGATGACCCAGAGCCCCAGCAGCCTGTCTGCCAG |
| Light Chain | | CGTGGGCGACAGAGTGACCATCACCTGTCAGGCCAGCGACT |
| | | ACATCTACCACTGGCTGGCCTGGTATCAGCAGAAGCCCGGC |
| | | AAGGCCCCCAAGCTGCTGATTAGCGGAGCCTCCAGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCATCCCCTG |
| | | GACCTTTGGCCAGGGCACCAAGCTGGAAATCAAGCGTACGG |
| | | TGGCCGCTCCCAGCGTGTTCATCTTCCCCCCCAGCGACGAG |
| | | CAGCTGAAGAGCGGCACCGCCAGCGTGGTGTGCCTGCTGAA |
| | | CAACTTCTACCCCCGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAAGCATAAGGTGT |
| | | ACGCCTGCGAGGTGACCCACCAGGGCCTGTCCAGCCCCGTG |
| | | ACCAAGAGCTTCAACAGGGGCGAGTGC |
| NEG319 | | |
| HCDR1 (Kabat) | 132 | SFWIT |
| HCDR2 (Kabat) | 133 | DIYPGGANTNYNEKLQG |
| HCDR3 (Kabat) | 134 | SPPQVGPFDY |
| HCDR1 (Chothia) | 135 | GYTFTSF |
| HCDR2 (Chothia) | 136 | YPGGAN |
| HCDR3 (Chothia) | 137 | SPPQVGPFDY |
| VH | 138 | QVQLVQSGAEVKKPGASVKVSCKASGYTFTSFWITWVRQAP |
| | | GQGLEWMGDIYPGGANTNYNEKLQGRVTLTVDTSTSTAYME |
| | | LRSLRSDDTAVYYCARSPPQVGPFDYWGQGTLVTVSS |
| DNA Encoding VH | 139 | CAGGTGCAGCTGGTGCAGTCTGGCGCCGAAGTGAAGAAACC |
| | | AGGCGCCAGCGTGAAGGTGTCCTGCAAGGCCAGCGGCTATA |
| | | CCTTCACCAGCTTTTGGATCACCTGGGTGCGCCAGGCCCCT |
| | | GGACAGGGACTGGAATGGATGGGCGACATCTACCCTGGCGG |
| | | CGCCAACACCAACTACAACGAGAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTACTACTG |
| | | CGCTAGAAGCCCTCCTCAGGTGGGCCCCTTCGATTATTGGG |
| | | GCCAGGGCACACTCGTGACCGTGTCCTCT |
| Heavy Chain | 140 | QVQLVQSGAEVKKPGASVKVSCKASGYTFTSFWITWVRQAP |
| | | GQGLEWMGDIYPGGANTNYNEKLQGRVTLTVDTSTSTAYME |
| | | LRSLRSDDTAVYYCARSPPQVGPFDYWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSREEMTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| DNA Encoding | 141 | CAGGTGCAGCTGGTGCAGTCTGGCGCCGAAGTGAAGAAACC |
| Heavy Chain | | AGGCGCCAGCGTGAAGGTGTCCTGCAAGGCCAGCGGCTATA |
| | | CCTTCACCAGCTTTTGGATCACCTGGGTGCGCCAGGCCCCT |
| | | GGACAGGGACTGGAATGGATGGGCGACATCTACCCTGGCGG |
| | | CGCCAACACCAACTACAACGAGAAGCTGCAGGGCAGAGTGA |
| | | CCCTGACCGTGGACACCAGCACCTCCACCGCCTACATGGAA |
| | | CTGCGGAGCCTGAGAAGCGACGACACCGCCGTGTACTACTG |
| | | CGCTAGAAGCCCTCCTCAGGTGGGCCCCTTCGATTATTGGG |
| | | GCCAGGGCACACTCGTGACCGTGTCCTCTGCTAGCACCAAG |
| | | GGCCCCAGCGTGTTCCCCCTGGCCCCCAGCAGCAAGAGCAC |
| | | CAGCGGCGGCACAGCCGCCCTGGGCTGCCTGGTGAAGGACT |
| | | ACTTCCCCGAGCCCGTGACCGTGTCCTGGAACAGCGGAGCC |
| | | CTGACCTCCGGCGTGCACACCTTCCCCGCCGTGCTGCAGAG |
| | | CAGCGGCCTGTACAGCCTGTCCAGCGTGGTGACAGTGCCCA |
| | | GCAGCAGCCTGGGCACCCAGACCTACATCTGCAACGTGAAC |
| | | CACAAGCCCAGCAACACCAAGGTGGACAAGAGAGTGGAGCC |
| | | CAAGAGCTGCGACAAGACCCACACCTGCCCCCCCTGCCCAG |
| | | CCCCAGAGCTGCTGGGCGGACCCTCCGTGTTCCTGTTCCCC |
| | | CCCAAGCCCAAGGACACCCTGATGATCAGCAGGACCCCCGA |
| | | GGTGACCTGCGTGGTGGTGGACGTGAGCCACGAGGACCCAG |
| | | AGGTGAAGTTCAACTGGTACGTGGACGGCGTGGAGGTGCAC |
| | | AACGCCAAGACCAAGCCCAGAGAGGAGCAGTACAACAGCAC |
| | | CTACAGGGTGGTGTCCGTGCTGACCGTGCTGCACCAGGACT |
| | | GGCTGAACGGCAAGGAATACAAGTGCAAGGTCTCCAACAAG |
| | | GCCCTGCCAGCCCCCATCGAAAAGACCATCAGCAAGGCCAA |
| | | GGGCCAGCCACGGGAGCCCCAGGTGTACACCCTGCCCCCCT |
| | | CCCGGGAGGAGATGACCAAGAACCAGGTGTCCCTGACCTGT |
| | | CTGGTGAAGGGCTTCTACCCCAGCGACATCGCCGTGGAGTG |
| | | GGAGAGCAACGGCCAGCCCGAGAACAACTACAAGACCACCC |
| | | CCCCAGTGCTGGACAGCGACGGCAGCTTCTTCCTGTACAGC |
| | | AAGCTGACCGTGGACAAGTCCAGGTGGCAGCAGGGCAACGT |
| | | GTTCAGCTGCAGCGTGATGCACGAGGCCCTGCACAACCACT |
| | | ACACCCAGAAGAGCCTGAGCCTGTCCCCCGGCAAG |
| LCDR1 (Kabat) | 142 | QASEYIINWLA |
| LCDR2 (Kabat) | 143 | GATGLET |
| LCDR3 (Kabat) | 144 | QQYWSIPWT |
| LCDR1 (Chothia) | 145 | SEYIINW |
| LCDR2 (Chothia) | 146 | GAT |
| LCDR3 (Chothia) | 147 | YWSIPW |
| VL | 148 | DIQMTQSPSSLSASVGDRVTITCQASEYIINWLAWYQQKPG |
| | | KAPKLLISGATGLETGVPSRFSGSGSGKDYTFTISSLQPED |
| | | IATYYCQQYWSIPWTFGQGTKLEIK |
| DNA Encoding VL | 149 | GACATCCAGATGACCCAGAGCCCCAGCAGCCTGTCTGCCAG |
| | | CGTGGGCGACAGAGTGACCATCACCTGTCAGGCCAGCGAAT |
| | | ACATCATAAACTGGCTGGCCTGGTATCAGCAGAAGCCCGGC |
| | | AAGGCCCCCAAGCTGCTGATTAGCGGAGCCACCGGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCATCCCCTG |
| | | GACCTTTGGCCAGGGCACCAAGCTGGAAATCAAG |
| Light Chain | 150 | DIQMTQSPSSLSASVGDRVTITCQASEYIINWLAWYQQKPG |
| | | KAPKLLISGATGLETGVPSRFSGSGSGKDYTFTISSLQPED |
| | | IATYYCQQYWSIPWTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| DNA Encoding | 151 | GACATCCAGATGACCCAGAGCCCCAGCAGCCTGTCTGCCAG |
| Light Chain | | CGTGGGCGACAGAGTGACCATCACCTGTCAGGCCAGCGAAT |
| | | ACATCATAAACTGGCTGGCCTGGTATCAGCAGAAGCCCGGC |
| | | AAGGCCCCCAAGCTGCTGATTAGCGGAGCCACCGGTCTGGA |
| | | AACCGGCGTGCCAAGCAGATTTTCCGGCAGCGGCTCCGGCA |
| | | AGGACTACACCTTCACCATCAGCTCCCTGCAGCCCGAGGAT |
| | | ATCGCCACCTACTACTGCCAGCAGTACTGGTCCATCCCCTG |
| | | GACCTTTGGCCAGGGCACCAAGCTGGAAATCAAGCGTACGG |
| | | TGGCCGCTCCCAGCGTGTTCATCTTCCCCCCCAGCGACGAG |
| | | CAGCTGAAGAGCGGCACCGCCAGCGTGGTGTGCCTGCTGAA |
| | | CAACTTCTACCCCCGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAAGCATAAGGTGT |
| | | ACGCCTGCGAGGTGACCCACCAGGGCCTGTCCAGCCCCGTG |
| | | ACCAAGAGCTTCAACAGGGGCGAGTGC |

Other antibodies of the invention include those where the amino acids or nucleic acids encoding the amino acids have been mutated, yet have at least 60, 65, 70, 75, 80, 85, 90, or 95 percent identity to the sequences described in Table 1. Some embodiments include mutant amino acid sequences wherein no more than 1, 2, 3, 4 or 5 amino acids have been mutated in the variable regions when compared with the variable regions depicted in the sequence described in Table 1, while retaining substantially the same antigen binding activity.

Since each of these antibodies can bind to ANGPTL4, the VH, VL, full-length light chain, and full-length heavy chain sequences (amino acid sequences and the nucleotide sequences encoding the amino acid sequences) can be “mixed and matched” to create other ANGPTL4-binding antibodies of the invention. Such “mixed and matched” ANGPTL4-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full-length heavy chain sequence from a particular full-length heavy chain/full length light chain pairing should be replaced with a structurally similar full-length heavy chain sequence. Likewise, a VL sequence from a particular VH/VL pairing should be replaced with a structurally similar VL sequence. Likewise a full-length light chain sequence from a particular full-length heavy chain/full-length light chain pairing should be replaced with a structurally similar full-length light chain sequence.

Accordingly, in one aspect, the invention provides an isolated antibody or antigen binding region thereof having: a heavy chain variable domain comprising an amino acid sequence selected from the group consisting of SEQ ID NOs: 13, 38, 58, 78, 98, 118, and 138, and a light chain variable domain comprising an amino acid sequence selected from the group consisting of SEQ ID NOs: 23, 48, 68, 88, 108, 128, and 148, wherein the antibody specifically binds to ANGPTL4 (e.g., human ANGPTL4).

More specifically, in certain aspects, the invention provides an isolated antibody or antigen binding region thereof having a heavy chain variable domain and a light chain variable domain comprising amino acid sequences selected from SEQ ID NOs: 13 and 23; 38 and 48; 58 and 68; 78 and 88; 98 and 108, 118 and 128, or 138 and 148, respectively.

In another aspect, the invention provides (i) an isolated antibody having: a full-length heavy chain comprising an amino acid sequence that has been optimized for expression in a mammalian cell selected from the group consisting of SEQ ID NOs: 15, 28, 40, 60, 80, 100, 120, and 140, and a full-length light chain comprising an amino acid sequence that has been optimized for expression in a mammalian cell selected from the group consisting of SEQ ID NOs: 25, 50, 70, 90, 110, 130, and 150; or (ii) a functional protein comprising an antigen binding portion thereof. More specifically, in certain aspects, the invention provides an isolated antibody or antigen binding region thereof having a heavy chain and a light chain comprising amino acid sequences selected from SEQ ID NOs: 15 and 25; 28 and 25; 40 and 50; 60 and 70; 80 and 90; 100 and 110; 120 and 130; or 140 and 150, respectively.

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme).

For example, under Kabat, the CDR amino acid residues of antibody FF1 in the heavy chain variable domain (VH) are numbered 31-35 (HCDR1), 50-66 (HCDR2), and 99-104 (HCDR3); and the CDR amino acid residues in the light chain variable domain (VL) are numbered 24-34 (LCDR1), 50-55 (LCDR2), and 89-97 (LCDR3). Under Chothia the CDR amino acids in the VH are numbered 26-32 (HCDR1), 52-57 (HCDR2), and 99-104 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-66 (HCDR2), and 90-104 (HCDR3) in human VH and amino acid residues 24-34 (LCDR1), 50-55 (LCDR2), and 89-97 (LCDR3) in human VL.

In another aspect, the present invention provides ANGPTL4 binding antibodies that comprise the heavy chain and light chain CDR1s, CDR2s, and CDR3s as described in Table 1, or combinations thereof. The amino acid sequences of the VH CDR1s of the antibodies are shown in SEQ ID NOs: 7, 32, 52, 72, 92, 112, and 132. The amino acid sequences of the VH CDR2s of the antibodies and are shown in SEQ ID NOs: 8, 33, 53, 73, 93, 113, and 133. The amino acid sequences of the VH CDR3s of the antibodies are shown in SEQ ID NOs: 9, 34, 54, 74, 94, 114, and 134. The amino acid sequences of the VL CDR1s of the antibodies are shown in SEQ ID NOs: 17, 42, 62, 82, 102, 122, and 142. The amino acid sequences of the VL CDR2s of the antibodies are shown in SEQ ID NOs: 18, 43, 63, 83, 103, 123, and 143. The amino acid sequences of the VL CDR3s of the antibodies are shown in SEQ ID NOs: 19, 44, 64, 84, 104, 124, and 144. These CDR regions are delineated using the Kabat system.

Alternatively, as defined using the Chothia system (Al-Lazikani et al., (1997) JMB 273,927-948), the amino acid sequences of the VH CDR1s of the antibodies are shown in SEQ ID NOs: 10, 35, 55, 75, 95, 115, and 135. The amino acid sequences of the VH CDR2s of the antibodies and are shown in SEQ ID NOs: 11, 36, 56, 76, 96, 116, and 136. The amino acid sequences of the VH CDR3s of the antibodies are shown in SEQ ID NOs: 12, 37, 57, 77, 97, 117, 117, and 137. The amino acid sequences of the VL CDR1s of the antibodies are shown in SEQ ID NOs: 20, 45, 65, 85, 105, 125, and 145. The amino acid sequences of the VL CDR2s of the antibodies are shown in SEQ ID NOs: 21, 46, 66, 86, 106, 126, and 146. The amino acid sequences of the VL CDR3s of the antibodies are shown in SEQ ID NOs: 22, 47, 67, 87, 107, 127, and 147.

Given that each of these antibodies can bind to ANGPTL4 and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched, although each antibody preferably contains a VH CDR1, 2 and 3 and a VL CDR1, 2 and 3 to create other ANGPTL4 binding molecules of the invention. Such “mixed and matched” ANGPTL4 binding antibodies can be tested using the binding assays known in the art and those described in the Examples (e.g., ELISAs, SET, Biacore). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present invention. In addition to the foregoing, in one embodiment, the antigen binding fragments of the antibodies described herein can comprise a VH CDR1, 2, and 3, or a VL CDR 1, 2, and 3, wherein the fragment binds to ANGPTL4 as a single variable domain.

In certain embodiments of the invention, the antibodies or antigen binding fragments thereof may have the heavy and light chain sequences of the Humanized antibodies described in Table 1. More specifically, the antibody or antigen binding fragments thereof may have the heavy and light sequence of NEG276, NEG276-LALA, NEG278, NEG310, NEG313, NEG315, NEG318, and NEG319.

In other embodiments of the invention the antibody or antigen binding fragment in that specifically binds ANGPTL4 comprises a heavy chain variable region CDR1, a heavy chain variable region CDR2, a heavy chain variable region CDR3, a light chain variable region CDR1, a light chain variable region CDR2, and a light chain variable region CDR3 as defined by Kabat and described in Table 1. In still other embodiments of the invention the antibody or antigen binding fragment in that specifically binds ANGPTL4 comprises a heavy chain variable region CDR1, a heavy chain variable region CDR2, a heavy chain variable region CDR3, a light chain variable region CDR1, a light chain variable region CDR2, and a light chain variable region CDR3 as defined by Chothia and described in Table 1.

In a specific embodiment, the invention includes an antibody that specifically binds to ANGPTL4 comprising a heavy chain variable region CDR1 of SEQ ID NO: 7; a heavy chain variable region CDR2 of SEQ ID NO: 8; a heavy chain variable region CDR3 of SEQ ID NO: 9; a light chain variable region CDR1 of SEQ ID NO: 17; a light chain variable region CDR2 of SEQ ID NO: 18; and a light chain variable region CDR3 of SEQ ID NO: 19.

In another specific embodiment, the invention includes an antibody that specifically binds to ANGPTL4 comprising a heavy chain variable region CDR1 of SEQ ID NO: 32; a heavy chain variable region CDR2 of SEQ ID NO: 33; a heavy chain variable region CDR3 of SEQ ID NO: 34; a light chain variable region CDR1 of SEQ ID NO: 42; a light chain variable region CDR2 of SEQ ID NO: 43; and a light chain variable region CDR3 of SEQ ID NO: 44.

In another specific embodiment, the invention includes an antibody that specifically binds to ANGPTL4 comprising a heavy chain variable region CDR1 of SEQ ID NO: 52; a heavy chain variable region CDR2 of SEQ ID NO: 53; a heavy chain variable region CDR3 of SEQ ID NO: 54; a light chain variable region CDR1 of SEQ ID NO: 62; a light chain variable region CDR2 of SEQ ID NO: 63; and a light chain variable region CDR3 of SEQ ID NO: 64.

In another specific embodiment, the invention includes an antibody that specifically binds to ANGPTL4 comprising a heavy chain variable region CDR1 of SEQ ID NO: 72; a heavy chain variable region CDR2 of SEQ ID NO: 73; a heavy chain variable region CDR3 of SEQ ID NO: 74; a light chain variable region CDR1 of SEQ ID NO: 82; a light chain variable region CDR2 of SEQ ID NO: 83; and a light chain variable region CDR3 of SEQ ID NO: 84.

In another specific embodiment, the invention includes an antibody that specifically binds to ANGPTL4 comprising a heavy chain variable region CDR1 of SEQ ID NO: 92; a heavy chain variable region CDR2 of SEQ ID NO: 93; a heavy chain variable region CDR3 of SEQ ID NO: 94; a light chain variable region CDR1 of SEQ ID NO: 102; a light chain variable region CDR2 of SEQ ID NO: 103; and a light chain variable region CDR3 of SEQ ID NO: 104.

In another specific embodiment, the invention includes an antibody that specifically binds to ANGPTL4 comprising a heavy chain variable region CDR1 of SEQ ID NO: 112; a heavy chain variable region CDR2 of SEQ ID NO: 113; a heavy chain variable region CDR3 of SEQ ID NO: 114; a light chain variable region CDR1 of SEQ ID NO: 122; a light chain variable region CDR2 of SEQ ID NO: 123; and a light chain variable region CDR3 of SEQ ID NO: 124.

In another specific embodiment, the invention includes an antibody that specifically binds to ANGPTL4 comprising a heavy chain variable region CDR1 of SEQ ID NO: 132; a heavy chain variable region CDR2 of SEQ ID NO: 133; a heavy chain variable region CDR3 of SEQ ID NO: 134; a light chain variable region CDR1 of SEQ ID NO: 142; a light chain variable region CDR2 of SEQ ID NO: 143; and a light chain variable region CDR3 of SEQ ID NO: 144.

In another specific embodiment, the invention includes an antibody that specifically binds to ANGPTL4 comprising a heavy chain variable region CDR1 of SEQ ID NO: 10; a heavy chain variable region CDR2 of SEQ ID NO: 11; a heavy chain variable region CDR3 of SEQ ID NO: 12; a light chain variable region CDR1 of SEQ ID NO: 20; a light chain variable region CDR2 of SEQ ID NO: 21; and a light chain variable region CDR3 of SEQ ID NO: 22.

In another specific embodiment, the invention includes an antibody that specifically binds to ANGPTL4 comprising a heavy chain variable region CDR1 of SEQ ID NO: 35; a heavy chain variable region CDR2 of SEQ ID NO: 36; a heavy chain variable region CDR3 of SEQ ID NO: 37; a light chain variable region CDR1 of SEQ ID NO: 45; a light chain variable region CDR2 of SEQ ID NO: 46; and a light chain variable region CDR3 of SEQ ID NO: 47.

In another specific embodiment, the invention includes an antibody that specifically binds to ANGPTL4 comprising a heavy chain variable region CDR1 of SEQ ID NO: 55; a heavy chain variable region CDR2 of SEQ ID NO: 56; a heavy chain variable region CDR3 of SEQ ID NO: 57; a light chain variable region CDR1 of SEQ ID NO: 65; a light chain variable region CDR2 of SEQ ID NO: 66; and a light chain variable region CDR3 of SEQ ID NO: 67.

In another specific embodiment, the invention includes an antibody that specifically binds to ANGPTL4 comprising a heavy chain variable region CDR1 of SEQ ID NO: 75; a heavy chain variable region CDR2 of SEQ ID NO: 76; a heavy chain variable region CDR3 of SEQ ID NO: 77; a light chain variable region CDR1 of SEQ ID NO: 85; a light chain variable region CDR2 of SEQ ID NO: 86; and a light chain variable region CDR3 of SEQ ID NO: 87.

In another specific embodiment, the invention includes an antibody that specifically binds to ANGPTL4 comprising a heavy chain variable region CDR1 of SEQ ID NO: 95; a heavy chain variable region CDR2 of SEQ ID NO: 96; a heavy chain variable region CDR3 of SEQ ID NO: 97; a light chain variable region CDR1 of SEQ ID NO: 105; a light chain variable region CDR2 of SEQ ID NO: 106; and a light chain variable region CDR3 of SEQ ID NO: 107.

In another specific embodiment, the invention includes an antibody that specifically binds to ANGPTL4 comprising a heavy chain variable region CDR1 of SEQ ID NO: 115; a heavy chain variable region CDR2 of SEQ ID NO: 116; a heavy chain variable region CDR3 of SEQ ID NO: 117; a light chain variable region CDR1 of SEQ ID NO: 125; a light chain variable region CDR2 of SEQ ID NO: 126; and a light chain variable region CDR3 of SEQ ID NO: 127.

In another specific embodiment, the invention includes an antibody that specifically binds to ANGPTL4 comprising a heavy chain variable region CDR1 of SEQ ID NO: 135; a heavy chain variable region CDR2 of SEQ ID NO: 136; a heavy chain variable region CDR3 of SEQ ID NO: 137; a light chain variable region CDR1 of SEQ ID NO: 145; a light chain variable region CDR2 of SEQ ID NO: 146; and a light chain variable region CDR3 of SEQ ID NO: 147.

In certain embodiments, the invention includes antibodies or antigen binding fragments that specifically bind to ANGPTL4 as described in Table 1. In a preferred embodiment, the antibody, or antigen binding fragment, that binds ANGPTL4 is NEG276, NEG276-LALA, NEG278, NEG310, NEG313, NEG315, NEG318, NEG319.

### Homologous Antibodies

In yet another embodiment, the present invention provides an antibody, or an antigen binding fragment thereof, comprising amino acid sequences that are homologous to the sequences described in Table 1, and the antibody binds to a ANGPTL4 protein (e.g., human and cynomolgus monkey ANGPTL4), and retains the desired functional properties of those antibodies described in Table 1.

For example, the invention provides an isolated antibody, or a functional antigen binding fragment thereof, comprising a heavy chain variable domain and a light chain variable domain, wherein the heavy chain variable domain comprises an amino acid sequence that is at least 80%, at least 90%, or at least 95% identical to an amino acid sequence selected from the group consisting of SEQ ID NOs: 13, 38, 58, 78, 98, 118, and 138; the light chain variable domain comprises an amino acid sequence that is at least 80%, at least 90%, or at least 95% identical to an amino acid sequence selected from the group consisting of SEQ ID NOs: 23, 23, 48, 68, 88, 108, 128, 148; and the antibody specifically binds to ANGPTL4 (e.g., human and cynomolgus monkey ANGPTL4). In certain aspects of the invention the heavy and light chain sequences further comprise HCDR1, HCDR2, HCDR3, LCDR1, LCDR2, and LCDR3 sequences as defined by Kabat, for example SEQ ID NOs: 7, 8, 9, 17, 18, and 19, respectively. In certain other aspects of the invention the heavy and light chain sequences further comprise HCDR1, HCDR2, HCDR3, LCDR1, LCDR2, and LCDR3 sequences as defined by Chothia, for example SEQ ID NOs: 10, 11, 12, 20, 21, and 22, respectively.

In other embodiments, the VH and/or VL amino acid sequences may be 50%, 60%, 70%, 80%, 90%, 95%, 96%, 97%, 98% or 99% identical to the sequences set forth in Table 1. In other embodiments, the VH and/or VL amino acid sequences may be identical except for an amino acid substitution in no more than 1, 2, 3, 4 or 5 amino acid positions. An antibody having VH and VL regions having high (i.e., 80% or greater) identity to the VH and VL regions of those described in Table 1 can be obtained by mutagenesis (e.g., site-directed or PCR-mediated mutagenesis) of nucleic acid molecules encoding SEQ ID NOs: 13, 38, 58, 78, 98, 118, 118, or 138 and SEQ ID NOs: 23, 48, 68, 88, 108, 128, or 148, respectively, followed by testing of the encoded altered antibody for retained function using the functional assays described herein.

In other embodiments, the full-length heavy chain and/or full-length light chain amino acid sequences may be 50% 60%, 70%, 80%, 90%, 95%, 96%, 97%, 98% or 99% identical to the sequences set forth in Table 1. An antibody having a full-length heavy chain and full-length light chain having high (i.e., 80% or greater) identity to the full-length heavy chains of any of SEQ ID NOs: 15, 28, 40, 60, 80, 100, 120, or 140, and full-length light chains of any of SEQ ID NOs: 25, 25, 50, 70, 90, 110, 130, or 150, can be obtained by mutagenesis (e.g., site-directed or PCR-mediated mutagenesis) of nucleic acid molecules encoding such polypeptides, followed by testing of the encoded altered antibody for retained function using the functional assays described herein.

In other embodiments, the full-length heavy chain and/or full-length light chain nucleotide sequences may be 60%, 70%, 80%, 90%, 95%, 96%, 97%, 98% or 99% identical to the sequences set forth in Table 1.

In other embodiments, the variable regions of heavy chain and/or the variable regions of light chain nucleotide sequences may be 60%, 70%, 80%, 90%, 95%, 96%, 97%, 98% or 99% identical to the sequences set forth in Table 1.

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

Additionally or alternatively, the protein sequences of the present invention can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In certain embodiments, an antibody of the invention has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the ANGPTL4-binding antibodies of the invention.

Accordingly, the invention provides an isolated antibody, or a antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 7, 32, 52, 72, 92, 112, and 132, and conservative modifications thereof; the heavy chain variable region CDR2 amino acid sequences are selected from the group consisting of SEQ ID NOs: 8, 33, 53, 73, 93, 113, and 133, and conservative modifications thereof; the heavy chain variable region CDR3 amino acid sequences are selected from the group consisting of SEQ ID NOs: 9, 34, 54, 74, 94, 114, and 134, and conservative modifications thereof; the light chain variable regions CDR1 amino acid sequences are selected from the group consisting of SEQ ID NOs: 17, 42, 62, 82, 102, 122, and 142, and conservative modifications thereof; the light chain variable regions CDR2 amino acid sequences are selected from the group consisting of SEQ ID NOs: 18, 43, 63, 83, 103, 123, and 143, and conservative modifications thereof; the light chain variable regions of CDR3 amino acid sequences are selected from the group consisting of SEQ ID NOs: 19, 44, 64, 84, 104, 124, and 144, and conservative modifications thereof; and the antibody or antigen binding fragments thereof specifically binds to ANGPTL4.

In other embodiments, the antibody of the invention is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the ANGPTL4 binding antibodies of the invention. Accordingly, the invention provides an isolated antibody optimized for expression in a mammalian cell consisting of a full-length heavy chain and a full-length light chain wherein the full length heavy chain has amino acid sequences selected from the group of SEQ ID NOs: 15, 28, 40, 60, 80, 100, 120, and 140, and conservative modifications thereof; and the full length light chain has amino acid sequences selected from the group of SEQ ID NOs: 25, 50, 70, 90, 110, 130, and 150, and conservative modifications thereof; and the antibody specifically binds to ANGPTL4 (e.g., human and cynomolgus monkey ANGPTL4).

Antibodies that Bind to the Same Epitope

The present invention provides antibodies that bind to the same epitope as the ANGPTL4 binding antibodies described in Table 1. Additional antibodies can therefore be identified based on their ability to compete (e.g., to competitively inhibit the binding of, in a statistically significant manner) with other antibodies of the invention in ANGPTL4 binding assays (such as those described in the Examples). The ability of a test antibody to inhibit the binding of antibodies of the present invention to a ANGPTL4 protein demonstrates that the test antibody can compete with that antibody for binding to ANGPTL4; such an antibody may, according to non-limiting theory, bind to the same or a related (e.g., a structurally similar or spatially proximal) epitope on the ANGPTL4 protein as the antibody with which it competes. In a certain embodiment, the antibody that binds to the same epitope on ANGPTL4 as the antibodies of the present invention is a humanized antibody. Such humanized antibodies can be prepared and isolated as described herein. As used herein, an antibody “competes” for binding when the competing antibody inhibits ANGPTL4 binding of an antibody or antigen binding fragment of the invention by more than 50% (for example, 80%, 85%, 90%, 95%, 98% or 99%) in the presence of an equimolar concentration of competing antibody.

In other embodiments the antibodies or antigen binding fragments of the invention bind to one or more epitopes of ANGPTL4. In some embodiments, the epitopes to which the present antibodies or antigen binding fragments bind are linear epitopes. In other embodiments, the epitopes to which the present antibodies or antigen binding fragments bind are non-linear, conformational epitopes.

### Engineered and Modified Antibodies

An antibody of the invention further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al).

Accordingly, another embodiment of the invention pertains to an isolated antibody, or an antigen binding fragment thereof, comprising a heavy chain variable region comprising CDR1 sequences having an amino acid sequence selected from the group consisting of SEQ ID NOs: 7, 32, 52, 72, 92, 112, and 132; CDR2 sequences having an amino acid sequence selected from the group consisting of SEQ ID NOs: 8, 33, 53, 73, 93, 113, and 133; CDR3 sequences having an amino acid sequence selected from the group consisting of SEQ ID NOs: 9, 34, 54, 74, 94, 114, and 134, respectively; and a light chain variable region having CDR1 sequences having an amino acid sequence selected from the group consisting of SEQ ID NOs: 17, 42, 62, 82, 102, 122, and 142; CDR2 sequences having an amino acid sequence selected from the group consisting of SEQ ID NOs: 18, 43, 63, 83, 103, 123, and 143; and CDR3 sequences consisting of an amino acid sequence selected from the group consisting of SEQ ID NOs: 19, 44, 64, 84, 104, 124, and 144, respectively. Thus, such antibodies contain the VH and VL CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the world wide web at mrc-cpe.cam.ac.uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. Mol. Biol. 227:776-798; and Cox, J. P. L. et al., 1994 Eur. J Immunol. 24:827-836; the contents of each of which are expressly incorporated herein by reference.

An example of framework sequences for use in the antibodies of the invention are those that are structurally similar to the framework sequences used by selected antibodies of the invention, e.g., consensus sequences and/or framework sequences used by monoclonal antibodies of the invention. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). Frameworks that can be utilized as scaffolds on which to build the antibodies and antigen binding fragments described herein include, but are not limited to VH1A, VH1B, VH3, Vk1, VI2, and Vk2. Additional frameworks are known in the art and may be found, for example, in the vBase data base on the world wide web at vbase.mrc-cpe.cam.ac.uk/index.php?& MMN_position=1:1.

Accordingly, an embodiment of the invention relates to isolated ANGPTL4 binding antibodies, or antigen binding fragments thereof, comprising a heavy chain variable region comprising an amino acid sequence selected from the group consisting of SEQ ID NOs: 13, 38, 58, 78, 98, 118, and 138, or an amino acid sequence having one, two, three, four or five amino acid substitutions, deletions or additions in the framework region of such sequences, and further comprising a light chain variable region having an amino acid sequence selected from the group consisting of SEQ ID NOs: 23, 48, 68, 88, 108, 128, and 148, or an amino acid sequence having one, two, three, four or five amino acid substitutions, deletions or additions in the framework region of such sequences.

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Accordingly, in another embodiment, the invention provides isolated ANGPTL4-binding antibodies, or antigen binding fragments thereof, consisting of a heavy chain variable region having a VH CDR1 region consisting of an amino acid sequence selected from the group having SEQ ID NOs: 7, 32, 52, 72, 92, 112, and 132, or an amino acid sequence having one, two, three, four or five amino acid substitutions, deletions or additions as compared to SEQ ID NOs: 7, 32, 52, 72, 92, 112, 112, and 132; a VH CDR2 region having an amino acid sequence selected from the group consisting of SEQ ID NOs: 8, 33, 53, 73, 93, 113, and 133, or an amino acid sequence having one, two, three, four or five amino acid substitutions, deletions or additions as compared to SEQ ID NOs: 8, 33, 53, 73, 93, 113, and 133; a VH CDR3 region having an amino acid sequence selected from the group consisting of SEQ ID NOs: 9, 34, 54, 74, 94, 114, 114, and 134, or an amino acid sequence having one, two, three, four or five amino acid substitutions, deletions or additions as compared to SEQ ID NOs: 9, 34, 54, 74, 94, 114, and 134; a VL CDR1 region having an amino acid sequence selected from the group consisting of SEQ ID NOs: 17, 42, 62, 82, 102, 122, and 142, or an amino acid sequence having one, two, three, four or five amino acid substitutions, deletions or additions as compared to SEQ ID NOs: 17, 42, 62, 82, 102, 122, and 142; a VL CDR2 region having an amino acid sequence selected from the group consisting of SEQ ID NOs: 18, 43, 63, 83, 103, 123, and 143, or an amino acid sequence having one, two, three, four or five amino acid substitutions, deletions or additions as compared to SEQ ID NOs: 18, 43, 63, 83, 103, 123, and 143; and a VL CDR3 region having an amino acid sequence selected from the group consisting of SEQ ID NOs: 19, 44, 64, 84, 104, 124, and 144, or an amino acid sequence having one, two, three, four or five amino acid substitutions, deletions or additions as compared to SEQ ID NOs: 19, 44, 64, 84, 104, 124, and 144.

Accordingly, in another embodiment, the invention provides isolated ANGPTL4-binding antibodies, or antigen binding fragments thereof, consisting of a heavy chain variable region having a VH CDR1 region consisting of an amino acid sequence selected from the group having SEQ ID NOs: 10, 35, 55, 75, 95, 115, and 135 or an amino acid sequence having one, two, three, four or five amino acid substitutions, deletions or additions as compared to SEQ ID NOs: 10, 35, 55, 75, 95, 115, and 135; a VH CDR2 region having an amino acid sequence selected from the group consisting of SEQ ID NOs: 11, 36, 56, 76, 96, 116, and 136 or an amino acid sequence having one, two, three, four or five amino acid substitutions, deletions or additions as compared to SEQ ID NOs: 11, 36, 56, 76, 96, 116, and 136; a VH CDR3 region having an amino acid sequence selected from the group consisting of SEQ ID NOs: 12, 37, 57, 77, 97, 117, and 137, or an amino acid sequence having one, two, three, four or five amino acid substitutions, deletions or additions as compared to SEQ ID NOs: 12, 37, 57, 77, 97, 117, and 137; a VL CDR1 region having an amino acid sequence selected from the group consisting of SEQ ID NOs: 20, 45, 65, 85, 105, 125, and 145, or an amino acid sequence having one, two, three, four or five amino acid substitutions, deletions or additions as compared to SEQ ID NOs: 20, 45, 65, 85, 105, 125, and 145; a VL CDR2 region having an amino acid sequence selected from the group consisting of SEQ ID NOs: 21, 46, 66, 86, 106, 126, and 146, or an amino acid sequence having one, two, three, four or five amino acid substitutions, deletions or additions as compared to SEQ ID NOs: 21, 46, 66, 86, 106, 126, and 146; and a VL CDR3 region having an amino acid sequence selected from the group consisting of SEQ ID NOs: 22, 47, 67, 87, 107, 127, and 147, or an amino acid sequence having one, two, three, four or five amino acid substitutions, deletions or additions as compared to SEQ ID NOs: 22, 47, 67, 87, 107, 127, and 147.

Grafting Antigen-Binding Domains into Alternative Frameworks or Scaffolds

A wide variety of antibody/immunoglobulin frameworks or scaffolds can be employed so long as the resulting polypeptide includes at least one binding region which specifically binds to ANGPTL4. Such frameworks or scaffolds include the 5 main idiotypes of human immunoglobulins, or fragments thereof, and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the invention pertains to generating non-immunoglobulin based antibodies using non-immunoglobulin scaffolds onto which CDRs of the invention can be grafted. Known or future non-immunoglobulin frameworks and scaffolds may be employed, as long as they comprise a binding region specific for the target ANGPTL4 protein. Known non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxibodies (Avidia, Inc., Mountain View, CA), Protein A (Affibody AG, Sweden), and affilin (gamma-crystallin or ubiquitin)(Scil Proteins GmbH, Halle, Germany).

The fibronectin scaffolds are based on fibronectin type Ill domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

The present invention provides fully human antibodies that specifically bind to a ANGPTL4 protein. Compared to the chimeric or humanized antibodies, the human ANGPTL4-binding antibodies of the invention have further reduced antigenicity when administered to human subjects.

### Camelid Antibodies

Antibody proteins obtained from members of the camel and dromedary (Camelus bactrianus and Calelus dromedarius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279:1256-1261; Dumoulin, M. et al., 2003 Nature 424:783-788; Pleschberger, M. et al. 2003 Bioconjugate Chem 14:440-448; Cortez-Retamozo, V. et al. 2002 Int J Cancer 89:456-62; and Lauwereys, M. et al. 1998 EMBO J 17:3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier. See U.S. patent application No. 20040161738 published Aug. 19, 2004. These features combined with the low antigenicity to humans indicate great therapeutic potential. Further, these molecules can be fully expressed in prokaryotic cells such as E. coli and are expressed as fusion proteins with bacteriophage and are functional.

Accordingly, a feature of the present invention is a camelid antibody or nanobody having high affinity for ANGPTL4. In certain embodiments herein, the camelid antibody or nanobody is naturally produced in the camelid animal, i.e., is produced by the camelid following immunization with ANGPTL4 or a peptide fragment thereof, using techniques described herein for other antibodies. Alternatively, the ANGPTL4-binding camelid nanobody is engineered, i.e., produced by selection for example from a library of phage displaying appropriately mutagenized camelid nanobody proteins using panning procedures with ANGPTL4 as a target as described in the examples herein. Engineered nanobodies can further be customized by genetic engineering to have a half life in a recipient subject of from 45 minutes to two weeks. In a specific embodiment, the camelid antibody or nanobody is obtained by grafting the CDRs sequences of the heavy or light chain of the human antibodies of the invention into nanobody or single domain antibody framework sequences, as described for example in PCT/EP93/02214.

### Bispecific Molecules and Multivalent Antibodies

In another aspect, the present invention features bispecific or multispecific molecules comprising a ANGPTL4-binding antibody, or a fragment thereof, of the invention. An antibody of the invention, or antigen-binding regions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for ANGPTL4 and a second binding specificity for a second target epitope. For example, the second target epitope is another epitope of ANGPTL4 different from the first target epitope.

Additionally, for the invention in which the bispecific molecule is multi-specific, the molecule can further include a third binding specificity, in addition to the first and second target epitope.

In one embodiment, the bispecific molecules of the invention comprise as a binding specificity at least one antibody, or an antibody fragment thereof, including, e.g., a Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778.

Diabodies are bivalent, bispecific molecules in which VH and VL domains are expressed on a single polypeptide chain, connected by a linker that is too short to allow for pairing between the two domains on the same chain. The VH and VL domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure VHA-VLB and VHB-VLA (VH-VL configuration), or VLA-VHB and VLB-VHA (VL-VH configuration) within the same cell. Most of them can be expressed in soluble form in bacteria. Single chain diabodies (scDb) are produced by connecting the two diabody-forming polypeptide chains with linker of approximately 15 amino acid residues (see Holliger and Winter, 1997 Cancer Immunol. Immunother., 45 (3-4): 128-30; Wu et al., 1996 Immunotechnology, 2 (1): 21-36). scDb can be expressed in bacteria in soluble, active monomeric form (see Holliger and Winter, 1997 Cancer Immunol. Immunother., 45 (34): 128-30; Wu et al., 1996 Immunotechnology, 2 (1): 21-36; Pluckthun and Pack, 1997 Immunotechnology, 3 (2): 83-105; Ridgway et al., 1996 Protein Eng., 9 (7): 617-21). A diabody can be fused to Fc to generate a “di-diabody” (see Lu et al., 2004 J. Biol. Chem., 279 (4): 2856-65).

Other antibodies which can be employed in the bispecific molecules of the invention are murine, chimeric and humanized monoclonal antibodies.

Bispecific molecules can be prepared by conjugating the constituent binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio) propionate (SPDP), and sulfosuccinimidyl 4-(N-maleimidomethyl)cyclohaxane-I-carboxylate (sulfo-SMCC)(see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78,118-132; Brennan et al., 1985 Science 229:81-83), and Glennie et al., 1987 J. Immunol. 139:2367-2375). Conjugating agents are SATA and sulfo-SMCC, both available from Pierce Chemical Co. (Rockford, IL).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb x mAb, mAb x Fab, Fab x F(ab′)₂ or ligand x Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present invention provides multivalent compounds comprising at least two identical or different antigen-binding portions of the antibodies of the invention binding to ANGPTL4. The antigen-binding portions can be linked together via protein fusion or covalent or non covalent linkage. Alternatively, methods of linkage have been described for the bispecific molecules. Tetravalent compounds can be obtained for example by cross-linking antibodies of the antibodies of the invention with an antibody that binds to the constant regions of the antibodies of the invention, for example the Fc or hinge region.

Trimerizing domain are described for example in Borean patent EP 1 012 280B1. Pentamerizing modules are described for example in PCT/EP97/05897.

Antibodies with Extended Half Life

The present invention provides for antibodies that specifically bind to ANGPTL4 protein which have an extended half-life in vivo.

Many factors may affect a protein's half life in vivo. For examples, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the half life of the antibodies of the present invention. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10)alkoxy- or aryloxy-polyethylene glycol or polyethylene glycol-maleimide. In certain embodiments, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies of the invention. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a nonnative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum halflife of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin (e.g., human serum albumin; HSA) in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622. In addition, in the context of a bispecific antibody as described above, the specificities of the antibody can be designed such that one binding domain of the antibody binds to ANGPTL4 while a second binding domain of the antibody binds to serum albumin, preferably HSA.

The strategies for increasing half life is especially useful in nanobodies, fibronectin-based binders, and other antibodies or proteins for which increased in vivo half life is desired.

### Antibody Conjugates

The present invention provides antibodies or fragments thereof that specifically bind to a ANGPTL4 protein recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antigen-binding fragment of an antibody described herein (e.g., a Fab fragment, Fd fragment, Fv fragment, F(ab)₂ fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88:10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16 (2): 76-82; Hansson, et al., 1999, J. Mol. Biol. 287:265-76; and Lorenzo and Blasco, 1998, Biotechniques 24 (2): 308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to a ANGPTL4 protein may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, CA, 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present invention or fragments thereof conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115 In, 113 In, 112 In, and 111 In), technetium (99Tc), thallium (201Ti), gallium (68Ga, 67Ga), palladium (103Pd), molybdenum (99 Mo), xenon (133Xe), fluorine (18F), 153Sm, 177 Lu, 159Gd, 149Pm, 140 La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54 Mn, 75Se, 113Sn, and 117Tin; and positron emitting metals using various positron emission tomographies, and noradioactive paramagnetic metal ions.

The present invention further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alph-emiters such as 213Bi or macrocyclic chelators useful for conjugating radiometal ions, including but not limited to, 131 In, 131 LU, 131Y, 131Ho, 131Sm, to polypeptides. In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4 (10): 2483-90; Peterson et al., 1999, Bioconjug. Chem. 10 (4): 553-7; and Zimmerman et al., 1999, Nucl. Med. Biol. 26 (8): 943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Methods of Producing Antibodies of the Invention

### Nucleic Acids Encoding the Antibodies

The invention provides substantially purified nucleic acid molecules which encode polypeptides comprising segments or domains of the ANGPTL4-binding antibody chains described above. Some of the nucleic acids of the invention comprise the nucleotide sequence encoding the heavy chain variable region shown in SEQ ID NO: 13, 38, 58, 78, 98, 118, or 138, and/or the nucleotide sequence encoding the light chain variable region shown in SEQ ID NO: 23, 48, 68, 88, 108, 128, or 148. In a specific embodiment, the nucleic acid molecules are those identified in Table 1. Some other nucleic acid molecules of the invention comprise nucleotide sequences that are substantially identical (e.g., at least 65, 80%, 95%, or 99%) to the nucleotide sequences of those identified in Table 1. When expressed from appropriate expression vectors, polypeptides encoded by these polynucleotides are capable of exhibiting ANGPTL4 antigen binding capacity.

Also provided in the invention are polynucleotides which encode at least one CDR region and usually all three CDR regions from the heavy or light chain of the ANGPTL4-binding antibody set forth above. Some other polynucleotides encode all or substantially all of the variable region sequence of the heavy chain and/or the light chain of the ANGPTL4-binding antibody set forth above. Because of the degeneracy of the code, a variety of nucleic acid sequences will encode each of the immunoglobulin amino acid sequences.

The nucleic acid molecules of the invention can encode both a variable region and a constant region of the antibody. Some of nucleic acid sequences of the invention comprise nucleotides encoding a heavy chain sequence that is substantially identical (e.g., at least 80%, 90%, or 99%) to the heavy chain sequence set forth in SEQ ID NO: 15, 28, 40, 60, 80, 100, 120, and 140. Some other nucleic acid sequences comprising nucleotide encoding a light chain sequence that is substantially identical (e.g., at least 80%, 90%, or 99%) to the light chain sequence set forth in SEQ ID NO: 25, 50, 70, 90, 110, 130, and 150.

The polynucleotide sequences can be produced by de novo solid-phase DNA synthesis or by PCR mutagenesis of an existing sequence (e.g., sequences as described in the Examples below) encoding a ANGPTL4-binding antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

Also provided in the invention are expression vectors and host cells for producing the ANGPTL4-binding antibodies described above. Various expression vectors can be employed to express the polynucleotides encoding the ANGPTL4-binding antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the ANGPTL4-binding polynucleotides and polypeptides in mammalian (e.g., human) cells include pThioHis A, B & C, pcDNA3.1/His, pEBVHis A, B & C, (Invitrogen, San Diego, CA), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a ANGPTL4-binding antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a ANGPTL4-binding antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted ANGPTL4-binding antibody sequences. More often, the inserted ANGPTL4-binding antibody sequences are linked to a signal sequences before inclusion in the vector. Vectors to be used to receive sequences encoding ANGPTL4-binding antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the ANGPTL4-binding antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express ANGPTL4-binding polypeptides of the invention. Insect cells in combination with baculovirus vectors can also be used.

In some preferred embodiments, mammalian host cells are used to express and produce the ANGPTL4-binding polypeptides of the present invention. For example, they can be either a hybridoma cell line expressing endogenous immunoglobulin genes (e.g., the 1D6.C9 myeloma hybridoma clone as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed including the CHO cell lines, various Cos cell lines, Hela cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP poIIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation: nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express ANGPTL4-binding antibody chains or binding fragments can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Generation of Monoclonal Antibodies of the Invention

Monoclonal antibodies (mAbs) can be produced by a variety of techniques, including conventional monoclonal antibody methodology e.g., the standard somatic cell hybridization technique of Kohler and Milstein, 1975 Nature 256:495. Many techniques for producing monoclonal antibody can be employed e.g., viral or oncogenic transformation of B lymphocytes.

Animal systems for preparing hybridomas include the murine, rat and rabbit systems. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In a certain embodiment, the antibodies of the invention are humanized antibodies. Such humanized antibodies directed against ANGPTL4 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368 (6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13:65-93, and Harding, F. and Lonberg, N., 1995 Ann. N. Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et at., 1993 International Immunology 5:647-656; Tuaillon et al., 1993 Proc. Natl. Acad. Sci. USA 94:3720-3724; Choi et al., 1993 Nature Genetics 4:117-123; Chen, J. et al., 1993 EMBO J. 12:821-830; Tuaillon et al., 1994 J. Immunol. 152:2912-2920; Taylor, L. et al., 1994 International Immunology 579-591; and Fishwild, D. et al., 1996 Nature Biotechnology 14:845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al; PCT Publication Nos. WO 92/03918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise ANGPTL4-binding antibodies of the invention. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise ANGPTL4-binding antibodies of the invention. For example, mice carrying both a human heavy chain transchromosome and a human light chain transchromosome, referred to as “TC mice” can be used; such mice are described in Tomizuka et al., 2000 Proc. Natl. Acad. Sci. USA 97:722-727. Furthermore, cows carrying human heavy and light chain transchromosome have been described in the art (Kuroiwa et al., 2002 Nature Biotechnology 20:889-894) and can be used to raise ANGPTL4-binding antibodies of the invention.

Humanized antibodies of the invention can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and U.S. Pat. No. 5,571,698 to Ladner et al; U.S. Pat. Nos. 5,427,908 and 5,580,717 to Dower et al; U.S. Pat. Nos. 5,969,108 and 6,172,197 to McCafferty et al; and U.S. Pat. Nos. 5,885,793; 6,521,404; 6,544,731; 6,555,313; 6,582,915 and 6,593,081 to Griffiths et al.

Humanized antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Framework or Fc Engineering

Engineered antibodies of the invention include those in which modifications have been made to framework residues within VH and/or VL, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252 L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered CIq binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chem. 276:6591-6604).

In still another embodiment, the glycosylation of an antibody is modified. For example, an aglycosylated antibody can be made (i.e., the antibody lacks glycosylation). Glycosylation can be altered to, for example, increase the affinity of the antibody for “antigen”. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta (1,4)—N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

### Methods of Engineering Altered Antibodies

As discussed above, the ANGPTL4-binding antibodies having VH and VL sequences or full length heavy and light chain sequences shown herein can be used to create new ANGPTL4-binding antibodies by modifying full length heavy chain and/or light chain sequences, VH and/or VL sequences, or the constant region(s) attached thereto. Thus, in another aspect of the invention, the structural features of a ANGPTL4-binding antibody of the invention are used to create structurally related ANGPTL4-binding antibodies that retain at least one functional property of the antibodies of the invention, such as binding to human ANGPTL4 and also inhibiting one or more functional properties of ANGPTL4 (e.g., inhibit ANGPTL4 binding to the ANGPTL4 receptor, inhibit ANGPTL4-dependent cell proliferation).

For example, one or more CDR regions of the antibodies of the present invention, or mutations thereof, can be combined recombinantly with known framework regions and/or other CDRs to create additional, recombinantly-engineered, ANGPTL4-binding antibodies of the invention, as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing a ANGPTL4-binding antibody consisting of a heavy chain variable region antibody sequence having a CDR1 sequence selected from the group consisting of SEQ ID NOs: 7, 32, 52, 72, 92, 112, and 132, a CDR2 sequence selected from the group consisting of SEQ ID NOs: 8, 33, 53, 73, 93, 113, and 133, and/or a CDR3 sequence selected from the group consisting of SEQ ID NOs: 9, 34, 54, 74, 94, 114, and 134; and a light chain variable region antibody sequence having a CDR1 sequence selected from the group consisting of SEQ ID NOs: 17, 42, 62, 82, 102, 122, and 142, a CDR2 sequence selected from the group consisting of SEQ ID NOs: 18, 43, 63, 83, 103, 123, and 143, and/or a CDR3 sequence selected from the group consisting of SEQ ID NOs: 19, 44, 64, 84, 104, 124, and 144; altering at least one amino acid residue within the heavy chain variable region antibody sequence and/or the light chain variable region antibody sequence to create at least one altered antibody sequence; and expressing the altered antibody sequence as a protein.

Accordingly, in another embodiment, the invention provides a method for preparing a ANGPTL4-binding antibody consisting of a heavy chain variable region antibody sequence having a CDR1 sequence selected from the group consisting of SEQ ID NOs: 10, 35, 55, 75, 95, 115, and 135, a CDR2 sequence selected from the group consisting of SEQ ID NOs: 11, 36, 56, 76, 96, 116, and 136, and/or a CDR3 sequence selected from the group consisting of SEQ ID NOs: 12, 37, 57, 77, 97, 117, and 137; and a light chain variable region antibody sequence having a CDR1 sequence selected from the group consisting of SEQ ID NOs: 20, 45, 65, 85, 105, 125, and 145, a CDR2 sequence selected from the group consisting of SEQ ID NOs: 21, 46, 66, 86, 106, 126, and 146, and/or a CDR3 sequence selected from the group consisting of SEQ ID NOs: 22, 47, 67, 87, 107, 127, and 147; altering at least one amino acid residue within the heavy chain variable region antibody sequence and/or the light chain variable region antibody sequence to create at least one altered antibody sequence; and expressing the altered antibody sequence as a protein.

Accordingly, in another embodiment, the invention provides a method for preparing a ANGPTL4-binding antibody optimized for expression in a mammalian cell consisting of: a full-length heavy chain antibody sequence having a sequence selected from the group of SEQ ID NOs: 15, 28, 40, 60, 80, 100, 120, and 140; and a full length light chain antibody sequence having a sequence selected from the group of 25, 50, 70, 90, 110, 130, and 150; altering at least one amino acid residue within the full length heavy chain antibody sequence and/or the full length light chain antibody sequence to create at least one altered antibody sequence; and expressing the altered antibody sequence as a protein. In one embodiment, the alteration of the heavy or light chain is in the framework region of the heavy or light chain.

The altered antibody sequence can also be prepared by screening antibody libraries having fixed CDR3 sequences or minimal essential binding determinants as described in US2005/0255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence(s) is one that retains one, some or all of the functional properties of the ANGPTL4-binding antibodies described herein, which functional properties include, but are not limited to, specifically binding to human, cynomolgus, rat, and/or mouse ANGPTL4; and the antibody inhibit ANGPTL4-dependent cell proliferation in a F36E and/or Ba/F3-ANGPTL4R cell proliferation assay.

In certain embodiments of the methods of engineering antibodies of the invention, mutations can be introduced randomly or selectively along all or part of an ANGPTL4-binding antibody coding sequence and the resulting modified ANGPTL4-binding antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

In certain embodiments of the invention antibodies have been engineered to remove sites of deamidation. Deamidation is known to cause structural and functional changes in a peptide or protein. Deamidation can result in decreased bioactivity, as well as alterations in pharmacokinetics and antigenicity of the protein pharmaceutical. (Anal Chem. 2005 Mar. 1; 77(5):1432-9).

In certain embodiments of the invention the antibodies have been engineered to increase pl and improve their drug-like properties. The pl of a protein is a key determinant of the overall biophysical properties of a molecule. Antibodies that have low pls have been known to be less soluble, less stable, and prone to aggregation. Further, the purification of antibodies with low pl is challenging and can be problematic especially during scale-up for clinical use. Increasing the pl of the anti-ANGPTL4 antibodies, or Fabs, of the invention improved their solubility, enabling the antibodies to be formulated at higher concentrations (>100 mg/ml). Formulation of the antibodies at high concentrations (e.g. >100 mg/ml) offers the advantage of being able to administer higher doses of the antibodies into eyes of patients via intravitreal injections, which in turn may enable reduced dosing frequency, a significant advantage for treatment of chronic diseases including cardiovascular disorders. Higher pls may also increase the FcRn-mediated recycling of the IgG version of the antibody thus enabling the drug to persist in the body for a longer duration, requiring fewer injections. Finally, the overall stability of the antibodies is significantly improved due to the higher pl resulting in longer shelf-life and bioactivity in vivo. Preferably, the pl is greater than or equal to 8.2.

The functional properties of the altered antibodies can be assessed using standard assays available in the art and/or described herein, such as those set forth in the Examples (e.g., ELISAs).

### Prophylactic and Therapeutic Uses

Antibodies that binds ANGPTL4 as described herein, can be used at a therapeutically useful concentration for the treatment of a disease or disorder associated with increased ANGPTL4 levels and/or activity by administering to a subject in need thereof an effective amount of the antibodies or antigen binding fragments of the invention. The present invention provides a method of treating ANGPTL4-associated cardiovascular disorders by administering to a subject in need thereof an effective amount of the antibodies of the invention. The present invention provides a method of treating ANGPTL4-associated cardiovascular disorders by administering to a subject in need thereof an effective amount of the antibodies of the invention.

The antibodies of the invention can be used, inter alia, to prevent treat, prevent, and improve ANGPTL4 associated conditions or disorders, including but not limited to any number of conditions or diseases in which the ANGPTL4 protein levels are aberrantly high and/or in which a reduction of ANGPTL4 protein levels is sought. These conditions include but are not limited to those involving lipid metabolism, such as hyperlipidemia, hyperlipoproteinemia and dyslipidemia, including atherogenic dyslipidemia, diabetic dyslipidemia, hypertriglyceridemia (e.g., severe hypertriglyceridemia (e.g., with TG>1000 mg/dL), hypertriglyceridemia associated with obesity, and Type V hypertriglyceridemia) hypercholesterolemia, chylomicronemia, mixed dyslipidemia (obesity, metabolic syndrome, diabetes, etc.), lipodystrophy, lipoatrophy, and other conditions caused by, e.g., decreased LPL activity and/or LPL deficiency, decreased LDL receptor activity and/or LDL receptor deficiency, altered ApoC2, ApoE deficiency, increased ApoB, increased production and/or decreased elimination of very low-density lipoprotein (VLDL), certain drug treatment (e.g., glucocorticoid treatment-induced dyslipidemia), any genetic predisposition, diet, life style, and the like.

Other ANGPTL4-associated diseases or disorders associated with or resulting from hyperlipidemia, hyperlipoproteinemia, and/or dyslipidemia, include, but are not limited to, cardiovascular diseases or disorders, such as atherosclerosis, aneurysm, hypertension, angina, stroke, cerebrovascular diseases, congestive heart failure, coronary artery diseases, myocardial infarction, peripheral vascular diseases, and the like; acute pancreatitis; nonalcoholic steatohepatitis (NASH); blood sugar disorders, such as diabetes; obesity, and the like.

The antibodies of the invention can also be used in combination with other agents for the prevention, treatment, or improvement of ANGPTL4 associated disorders. For example, statin therapies may be used in combination with the ANGPTL4 antibodies and antigen binding fragments of the invention for the treatment of patients with triglyceride-related disorders.

### Pharmaceutical Compositions

The invention provides pharmaceutical compositions comprising the ANGPTL4-binding antibodies (intact or binding fragments) formulated together with a pharmaceutically acceptable carrier. The compositions can additionally contain one or more other therapeutic agents that are suitable for treating or preventing, for example, cardiovascular disorders. Pharmaceutically acceptable carriers enhance or stabilize the composition, or can be used to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that administration be intravitreal, intravenous, intramuscular, intraperitoneal, or subcutaneous, or administered proximal to the site of the target. The pharmaceutically acceptable carrier should be suitable for intravitreal, intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the ANGPTL4-binding antibody is employed in the pharmaceutical compositions of the invention. The ANGPTL4-binding antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the antibodies of the invention employed in the pharmaceutical composition at levels lower than that required to achieve the desired therapeutic effect and gradually increase the dosage until the desired effect is achieved. In general, effective doses of the compositions of the present invention, for the treatment of a cardiovascular disorders described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy. For systemic administration with an antibody, the dosage ranges from about 0.0001 to 100 mg/kg, and more usually 0.01 to 15 mg/kg, of the host body weight. For intravitreal administration with an antibody, the dosage may range from 0.1 mg/eye to 5 mg/eye. For example, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3.0 mg/ml, 3.1 mg/ml, 3.2 mg/ml, 3.3 mg/ml, 3.4 mg/ml, 3.5 mg/ml, 3.6 mg/ml, 3.7 mg/ml, 3.8 mg/ml, 3.9 mg/ml, 4.0 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, or 5.0 mg/ml. An exemplary treatment regime entails systemic administration once per every two weeks or once a month or once every 3 to 6 months. An exemplary treatment regime entails systemic administration once per every two weeks or once a month or once every 3 to 6 months, or as needed (PRN).

Antibody is usually administered on multiple occasions. Intervals between single dosages can be weekly, monthly or yearly. Intervals can also be irregular as indicated by measuring blood levels of ANGPTL4-binding antibody in the patient. In addition alternative dosing intervals can be determined by a physician and administered monthly or as necessary to be efficacious. In some methods of systemic administration, dosage is adjusted to achieve a plasma antibody concentration of 1-1000 μg/ml and in some methods 25-500 g/ml. Alternatively, antibody can be administered as a sustained release formulation, in which case less frequent administration is required. Dosage and frequency vary depending on the half-life of the antibody in the patient. In general, humanized antibodies show longer half life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

The following examples are provided to further illustrate the invention but not to limit its scope. Other variants of the invention will be readily apparent to one of ordinary skill in the art and are encompassed by the appended claims.

### Example 1: Preparation of Purified Recombinant Human ANGPTL4 for Use as Antigen and in Antibody Characterization Experiments

A nucleic acid sequence encoding full-length human ANGPTL4 polypeptide (amino acids 26-406, matching NCBI sequence NM_139314.2) with N-terminal signal peptide from human IgG-kappa (MKTFILLLWVLLLWVIFLLPGATA)(SEQ ID NO: 152), and C-terminal FLAG epitope (DYKDDDDKH)(SEQ ID NO: 153), hexahistidine purification tag (HHHHHH) (SEQ ID NO: 154), and Avi tag (i.e., BirA biotinylation sequence GGGLNDIFEAQKIEWHE) (SEQ ID NO: 155) was subcloned into the mammalian cell expression vector pRS5a to generate the plasmid pRS-Ikk-hANGPTL4 (26-406)-FLAG-6HIS-Avi containing a 20 amino acid IKK signal sequence followed by amino acids 26-406 of human ANGPTL4 with carboxyl-terminal Flag, 6HIS, and Avi tags (Table 2, SEQ ID NO: 156).

For some preparations, the following procedures were used to express, purify, and biotinylate human ANGPTL4 protein (Method 1): Suspension-adapted HEK293T cells were cultured in serum-free FreeStyle 293 expression medium (Life Technologies, catalog number 12338-018) and transfected with the plasmid pRS-Ikk-hANGPTL4 (26-406)-FLAG-His6-Avi using polyethyleneimine transfection reagent (Polysciences, catalog number 23966). Five hours after transfection, heparin (Alfa Aesar, catalog number A16198) was added to the culture medium to a final concentration of 0.5 mg/ml. The cells were then cultured for 72-96 hours and the cell culture supernatant was then harvested by centrifugation at 4° C. and sterile-filtered using a 0.22 μm filter (Thermo, catalog number 567-0010). The filtered cell culture supernatant was then concentrated to about 100 ml by tangential flow filtration. The concentrated supernatant was diluted to a volume of 1 Liter with TBS-glycerol buffer (50 mM Tris-HCl, 150 mM NaCl, and 15% glycerol, pH 7.4), and the sample was concentrated to about 200 ml by tangential flow filtration. Anti-Flag M2 agarose resin (Sigma, catalog number 220102-177) pre-equilibrated with TBS-glycerol buffer was then added to the sample, and the resulting solution was gently mixed for 1 hr at 4° C. The agarose resin was then washed 5 times with 25 ml TBS-glycerol, and the bound ANGPTL4 protein was eluted with 20 ml TBS-glycerol containing 0.2 mg/ml Flag peptide (Sigma, catalog number 220176-317). Peroxide-free Tween-20 (AppliChem, catalog number A1284,0025 was added to the eluted protein solution to a final concentration of 0.1%, and the resulting solution was loaded onto a 5 ml HiTrap heparin column (GE Lifesciences, catalog number 17-0407-01) that was pre-equilibrated in TBS-glycerol containing 0.1% Tween-20 (Buffer A). The column was washed with 50 ml Buffer A, followed by 50 ml Buffer A containing 300 mM NaCl. ANGPTL4 protein was then eluted with 20 ml Buffer A containing 600 mM NaCl. The eluted protein was concentrated using a centrifugal concentrator with a 30 kDa molecular weight cutoff (Amicon Ultra, catalog number UFC903024). The purity of the purified ANGPTL4 protein as assessed by SDS-PAGE was >90%.

For some applications, ANGPTL4 proteins were site-specifically biotinylated on the C-terminal Avi tag using 10 μg purified biotin-protein ligase (BirA)(Avidity) per mg of ANGPTL4. The buffer was supplemented with final concentrations of 10 mM ATP, 10 mM magnesium acetate, and 0.5 M d-biotin. The reaction mixture was incubated for 2 hr at 30° C. and then overnight at 4° C., then loaded onto a HiLoad Superdex 200 column (26 mm×600 mm)(GE Lifesciences, catalog number 28-9893-36) that was equilibrated in Buffer A. Fractions from the Superdex 200 column were analyzed using SDS-PAGE, and ANGPTL4 containing fractions were pooled and concentrated using a centrifugal concentrator.

For other preparations, the following procedures were used to express, purify, and biotinylate the human ANGPTL4 protein (Method 2). Plasmid pRS-Ikk-hANGPTL4 (26-406)C-Flag6HisAvi was transiently transfected into HEK293T cells using standard polyethylenimine (PEI) transfection methods. Cells were propagated in suspension culture in Freestyle 293 expression media and transfection was carried out at 1×10⁶ cells/ml final cell concentration in 4 liters media using 1 liter flasks. Five hours after transfection, heparin at a final concentration of 500 μg/ml was added. Cells were grown at 37° C. and 5% CO₂ for 72 hr. Cells were then pelleted by centrifugation, and the supernatant passed through a 0.22 μm sterile filter. The clarified supernatant was concentrated and buffer exchanged into Buffer B (50 mM Tris-HCl, 150 mM NaCl, 10% glycerol, 10 mM imidazole, pH 7.4) using tangential flow filtration (TFF). The concentrated sample was then passed over a 5 ml Ni-NTA affinity column equilibrated with Buffer C (50 mM Tris.HCl, 150 mM NaCl, 10% glycerol, 10 mM imidazole, 0.1% n-octyl-β-maltoside, pH 7.4). After loading the sample, the column was washed with the same buffer until baseline absorbance at 280 nm was reached. The bound ANGPTL4 protein was then eluted by using a gradient of imidazole (10 mM to 500 mM). Elution fractions that contained human ANGPTL4 were pooled, concentrated using an Amicon concentrator (molecular weight cut-off 10 kD), buffered-exchanged using PD-10 columns into storage buffer (50 mM Tris-HCl, 150 mM NaCl, 15% Glycerol, pH 7.4), aliquoted, flash frozen in liquid nitrogen, and stored at −80° C. The purity of the purified human ANGPTL4 protein as assessed by SDS-PAGE was >90%.

For some applications, purified ANGPTL4 protein prepared as described above was site-specifically biotinylated as follows: purified protein in 50 mM Bicine, pH 8.3 buffer at a final concentration of approximately 1 mg/mL was incubated in the presence of 10 mM ATP, 10 mM magnesium acetate, 0.1 mM biotin, and BirA biotin ligase (Avidity) at 30° C. for 1 hr and then at 4° C. overnight. The protein was then concentrated using an Amicon concentrator (molecular weight cut-off 10 kD), buffer-exchanged using PD-10 columns into storage buffer (50 mM Tris-HCl, 150 mM NaCl, 15% glycerol, pH 7.4), aliquoted, flash frozen in liquid nitrogen, and stored at −80° C.

### Example 2: Preparation of Purified Recombinant Human ANGPTL4 N-Terminal Coiled-Coil Domain Protein for Use in Antibody Characterization Experiments

Expression, purification, and biotinylation of the N-terminal coiled coil domain of human ANGPTL4 (amino acids 26-161) was carried out using essentially the same methods described for human full-length human ANGPTL4 in Example 1, Method 2. The sequence of the purified human ANGPTL4 N-terminal domain protein is shown in Table 2 (SEQ ID NO: 157).

TABLE 2
Amino acid sequences of human ANGPTL4(26-406)-FLAG-His6-Avi (the
signal peptide is highlighted by underlining, and the N-terminal QP
sequence after the signal peptide and the C-terminal FLAG-His6-Avi
sequences are highlighted with italics)

| | SEQ ID | |
|---|---|---|
| Construct | NO. | Amino Acid Sequence |
| Human | 156 | MKTFILLLWVLLLWVIFLLPGATAQPGPVQSKSPRFASWDEMNVLAH |
| ANGPTL4(26- | | GLLQLGQGLREHAERTRSQLSALERRLSACGSACQGTEGSTDLPLAP |
| 406)-FLAG- | | ESRVDPEVLHSLQTQLKAQNSRIQQLFHKVAQQQRHLEKQHLRIQHL |
| His6-Avi | | QSQFGLLDHKHLDHEVAKPARRKRLPEMAQPVDPAHNVSRLHRLPRD |
| | | CQELFQVGERQSGLFEIQPQGSPPFLVNCKMTSDGGWTVIQRRHDGS |
| | | VDFNRPWEAYKAGFGDPHGEFWLGLEKVHSITGDRNSRLAVQLRDWD |
| | | GNAELLQFSVHLGGEDTAYSLQLTAPVAGQLGATTVPPSGLSVPFST |
| | | WDQDHDLRRDKNCAKSLSGGWWFGTCSHSNLNGQYFRSIPQQRQKLK |
| | | KGIFWKTWRGRYYPLQATTMLIQPMAAEAASDYKDDDDEHHHHHHGG |
| | | GLNDIFEAQKIEWHE |
| Human | 157 | MKTFILLLWVLLLWVIFLLPGATAQPGPVQSKSPRFASWDEMNVLAH |
| ANGPTL4(26- | | GLLQLGQGLREHAERTRSQLSALERRLSACGSACQGTEGSTDLPLAP |
| 161)-FLAG- | | ESRVDPEVLHSLQTQLKAQNSRIQQLFHKVAQQQRHLEKQHLRIQHL |
| His6-Avi | | QSQFGLLDHKHLDHEVAKPARRKRLPEMAQPVDPAHNVSRLHRLPRD |
| | | YKDDDDEHHHHHHDYKDDDDEHHHHHHGGGLNDIFEAQKIEWHE |

### Example 3: Preparation and Screening of Monoclonal Antibodies

Recombinant human ANGPTL4 protein was prepared in-house as described in Example 1, and was used as immunogen for the generation of anti-ANGPTL4 hybridoma clones. Bcl-2 transgenic mice were immunized with recombinant human ANGPTL4 according to a standard rapid immunization protocol. Hybridomas were generated by using a standard electrofusion-based method.

CHO-K1PD cells stably expressing human ANGPTL4 fused to a transmembrane domain were generated using standard methods. Due to the presence of the transmembrane domain, these cells display ANGPTL4 on the cell surface. Therefore, binding of antibodies to ANGPTL4 on the surface of these cells can be detected using flow cytometry.

Hybridoma supernatants were screened by detecting binding of antibodies present in the supernatant to human ANGPTL4 expressed on the surface of CHO-K1PD cells. Binding of antibodies to the cells was detected using a fluorescently labeled anti-mouse secondary antibody and flow cytometry. Parental CHO-K1PD cells that do not express ANGPTL4 were used as a negative control. For hybridomas that bound to ANGPTL4, antibodies were purified from cell supernatants using standard methods, and the resulting enriched supernatant was tested in the flow cytometry assay with CHO-K1PD/ANGPTL4 and CHO-K1PD-Parental cells.

ANGPTL4 antibody titers in hybridoma supernatants were determined by using a standard direct ELISA assay, in which recombinant human ANGPTL4 protein was immobilized on the surface of the ELISA plate. Confirmed positive hybridomas were subcloned, and the sequences of the monoclonal antibodies produced by these hybridomas was determined using standard methods.

The monoclonal antibodies 14P18, 17B1, 19C16 and 37P1 were subsequently shown to inhibit human ANGPTL4 mediated inhibition of human lipoprotein lipase using methods described in Example 7 below. The nucleotide and amino acid sequences of the heavy and light chain variable regions of 14P18, 17B1, 19C16 and 37P1 were determined using standard methods.

### Example 4: Humanization of Monoclonal Antibodies

The process of humanization is well described in the art (Jones, et al 1986, Queen, et al 1989, Riechmann, et al 1988, Verhoeyen, Milstein and Winter 1988). The term humanization is described as the transfer of the antigen-binding site of a non-human antibody, e.g., a murine derived antibody, to a human acceptor framework, e.g., a human germline sequence (Retter, et al 2005). The main rationale for humanizing an antibody is to minimize the risk of developing an immunogenic response towards the antibody when the antibody is administered as a therapeutic in humans (Rebello, et al 1999).

The antigen-binding site comprises the complementary determining regions (CDRs) (Chothia and Lesk 1987, Kabat, et al 1991) and positions in the framework region of the variable domains (VL and VH) that directly or indirectly affect binding. Framework residues that may directly affect binding can, for example, be found in the so called “outer” loop region located between CDR2 and CDR3. Residues that indirectly affect binding are for example found at so called Vernier Zones (Foote and Winter 1992). They are thought to support CDR conformation. Those positions outside the CDRs are taken into account when choosing a suitable acceptor framework to minimize the number of deviations of the final humanized antibody to the human germline acceptor sequence in the framework regions.

### Example 5: Antibody Sequence Optimization and Affinity Maturation

Certain amino acid sequence motifs are known to undergo post-translational modification (PTM) such as glycosylation (e.g., NxS/T, where x is any amino acid except P), oxidation of free cysteines, deamidation (e.g., deamidation of N in NG sequences) or isomerization (e.g., at DG sequences). If present in the CDR regions, those motifs are ideally removed by site-directed mutagenesis in order to increase product homogeneity.

The process of affinity maturation is well described in the art. Among many display systems, phage display (Smith 1985) and display on eukaryotic cells such as yeast (Boder and Wittrup 1997) are the most commonly applied systems to select for antibody-antigen interaction. Advantages of these display systems are that they are suitable for a wide range of antigens and that the selection stringency can be easily adjusted. In phage display, scFv or Fab fragments can be displayed and in yeast display scFv, Fab or full-length IgG can be displayed. These commonly applied methods allow selection of desired antibody variants from larger libraries with diversities of more than 1×10⁷. Libraries with smaller diversity (e.g. 1,000) may be screen by micro-expression and ELISA.

Non-targeted or random antibody variant libraries can be generated for example by error-prone PCR (Cadwell and Joyce 1994) and provide a very simple, but sometimes limited approach. Another strategy is the CDR directed diversification of an antibody candidate. One or more positions in one or more CDRs can be targeted specifically using for example degenerate oligonucleotides (Thompson, et al 1996), trinucleotide mutagenesis (TRIM)(Kayushin, et al 1996), or any other approach known to the art.

### Example 6: Expression and Purification of Humanized Antibodies

DNA sequences coding for humanized VL and VH domains were ordered at GeneArt (Life Technologies, Inc., Regensburg, Germany) including codon optimization for Homo Sapiens. Sequences coding for VL and VH domains were subcloned by cutting and pasting from the GeneArt derived vectors into expression vectors suitable for expression and secretion by mammalian cells. The heavy and light chains were cloned into individual expression vectors to allow co-transfection. Elements of the expression vector include a promoter (Cytomegalovirus (CMV) enhancer-promoter), a signal sequence to facilitate secretion, a polyadenylation signal and transcription terminator (from the Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

Human Embryonic Kidney cells constitutively expressing the SV40 large T antigen (HEK293-T ATCC11268) are one of the preferred host cell lines for transient expression of humanized and/or optimized IgG proteins. Transfection is performed using PEI (Polyethylenimine, MW 25,000 linear, Polysciences, USA, catalog number 23966) as transfection reagent. The PEI stock solution is prepared by carefully dissolving 1 g of PEI in 900 ml cell culture grade water at room temperature (RT). To facilitate dissolution of PEI, the solution is acidified by addition of HCl to pH 3-5, followed by neutralization with NaOH to a final pH of 7.05. Finally, the volume is adjusted to 1 L and the solution is filtered through a 0.22 μm filter, aliquoted and frozen at −80° C. until further use. HEK 293T cells are cultivated using a Novartis proprietary serum-free culture medium for transfection and propagation of the cells, and ExCell VPRO serum-free culture medium (SAFC Biosciences, USA, Cat. No. 24561C) as production/feed medium. Cells prepared for transient transfections are cultivated in suspension culture. For small scale (<5 L) transfections, cells are grown in Corning shake flasks (Corning, Tewksbury, MA) on an orbital shaker (100-120 rpm) in a humidified incubator at 5% CO₂ (seed flasks). Cells in the seed cultures should be maintained in the exponential growth phase (cell densities between 5×10⁵/ml and 3×10⁶/ml) and display a viability of >90% for transfection. For small scale (<5 L) transfection an aliquot of cells is taken out of the seed cultures and adjusted to 1.4×10⁶ cells/ml in 36% of the final volume with Novartis serum-free culture medium. The DNA solution (Solution 1:0.5 mg of heavy chain and 0.5 mg of light chain expression plasmid for a 1 L transfection) is prepared by diluting the DNA to 1 mg/l (final volume) in 7% of the final culture volume followed by gentle mixing. To prevent bacterial contamination, this solution is filtered using a 0.22 μm filter (e.g., Millipore Stericup). Then 3 mg/L (final volume) of PEI solution is also diluted in 7% of final culture volume and mixed gently (Solution 2). Both solutions are incubated for 5-10 min at room temperature (RT). Thereafter solution 2 is added to solution 1 with gentle mixing and incubated for another 5-15 minutes at room temperature. The transfection mix is then added to the cells and the cultivation of cells is continued for 4 to 6 hours. Finally, the remaining 50% of total production volume is achieved by addition of ExCell® VPRO serum-free culture medium. The cell cultivation is continued for eleven days post-transfection.

The culture is harvested by centrifugation at 4500 rpm for 20 minutes at 4° C. (Heraeus®, Multifuge 3 S—R, Thermo Scientific, Rockford, IL). The cell supernatant recovered is sterile-filtered through a stericup filter (0.22 μm) and stored at 4° C. until further processing. Purification was performed on an “ÄKTA 100 Explorer Air” chromatography system at 4° C. in a cooling cabinet, using a freshly sanitized (0.25 M NaOH) HiTrap 5 ml Protein A MabSelect® SuRe column. The column was equilibrated with 5 column volumes of phosphate buffered saline (PBS, Gibco, Life Technologies, Carlsbad, CA), and then the sterile-filtered supernatant was loaded at 4.0 ml/min. The column was washed with 13 column volumes of PBS. Antibody was then eluted with 5 column volumes of 50 mM citrate, 70 mM NaCl, pH 3.2. The eluate was collected in 3 ml fractions and adjusted to pH 7 with 1 M Tris-HCl, pH 10. The antibody containing fractions were pooled and sterile-filtered (Millipore Steriflip, 0.22 μm), the OD 280 nm was measured using a spectrophotometer (NanoDrop ND-1000), and the protein concentration was calculated based on the OD 280 and the molar extinction coefficient which was calculated based on the protein sequence. The eluate was tested for aggregation by size exclusion chromatography with multi-angle light scattering detector (SEC-MALS) and purity was assessed by gel electrophoresis (SDS-PAGE), endotoxin assay (LAL) and mass spectrometry (MS). For the second purification step, if needed, antibody from the first purification was loaded onto a freshly sanitized (0.5 M NaOH) gel viltration column (Hi Load 16/60 Superdex 200, 120 mL, GE-Healthcare). The column was equilibrated with PBS and the run was done with PBS buffer at a flow rate of 1 ml/min. The eluate was collected in 1.2 ml fractions. Antibody containing proteins were pooled, and the resulting purified antibody analyzed as described for the first purification step.

Using the methods described above, the following humanized antibodies were prepared, expressed and purified: NEG276, NEG276-LALA, NEG278, NEG310, NEG318, NEG318-LALA, NEG319, NEG313, and NEG315. The framework and parental antibodies for these humanized antibodies is shown in Table 3, and the nucleotide and amino acid sequences are shown in Table 1. All humanized antibodies were prepared as human IgG1 antibodies, except for NEG276-LALA and NEG318-LALA, which were prepared using a modified Fc region (human IgG1-LALA) in which the Leu234-Leu235 sequence in the heavy chain is replaced by Ala234-Ala235. Human IgG1-LALA antibodies are known to have reduced antibody effector function compared to wild-type human IgG1 antibodies.

TABLE 3
Humanized ANGPTL4 antibodies of the invention.

| | VH | VL | | Parental |
|---|---|---|---|---|
| Antibody | SEQ ID NO. | SEQ ID NO. | Framework | Antibody |
| NEG276 | 13 | 23 | hIgG1/kappa | 19C16 |
| NEG276- | 13 | 23 | hIgG1-LALA/ | 19C16 |
| LALA | | | kappa | |
| NEG278 | 38 | 48 | hIgG1/kappa | 19C16 |
| NEG310 | 58 | 68 | hIgG1/kappa | 17B1 |
| NEG313 | 78 | 88 | hIgG1/kappa | 37P1 |
| NEG315 | 98 | 108 | hIgG1/kappa | 37P1 |
| NEG318 | 118 | 128 | hIgG1/kappa | 14P18 |
| NEG319 | 138 | 148 | hIgG1/kappa | 14P18 |

### Example 7: Human Lipoprotein Lipase Assay

HEK 293T cells cultured in FreeStyle expression medium (Invitrogen) were transfected with a mammalian expression plasmid encoding full-length human lipoprotein lipase (LPL) polypeptide (matching NCBI sequence NM_000237.2) using a standard polyethyleneimine (PEI) transfection method. At 24 hours after transfection, heparin was added to the culture medium to a final concentration of 3 U/ml, to enhance release of secreted hLPL from the cell surface. At 60 hours post-transfection, the culture medium was collected, filtered using a 0.2 μm filter, and glycerol was added to a final concentration of 10% v/v. The resulting solution was loaded onto a 5 ml Heparin Sepharose HiTrap column (GE) which had been pre-equilibrated with Buffer A (50 mM Tris-HCl, 200 mM NaCl, 10% v/v glycerol, pH7.2). The column was washed with Buffer A, and human LPL protein was then eluted with step gradients of 500 mM NaCl, 1 M NaCl, and 2 M NaCl in Buffer A. The purest and most catalytically active human LPL eluted at 2 M NaCl. Aliquots of purified human LPL were flash-frozen and stored at −80° C. until use.

The following protocol was used to assess the ability of antibodies of the invention to block ANGPTL4 inhibition of human lipoprotein lipase. The 384-well assay plate (Corning, catalog number 3573) and sample plate (Greiner Bio-one, catalog number 781201) were washed with 1% bovine serum albumin (BSA)(0.1 ml per well) for 30 min at room temperature. The plates were then washed twice with 0.05% Tween-20 solution.

ANGPTL4 antibody in 100 mM HEPES, pH 7.0 (20 μl per well, serial dilution with final assay concentrations ranging from 0.02 to 500 nM) was added to the sample plate, followed by 20 μl human ANGPTL4 protein (10 nM final assay concentration) in Assay Buffer (100 mM HEPES, 2 mM MgCl₂, pH 7.0), and the plate was incubated for 20 min at room temperature with gentle shaking. Lipoprotein lipase diluted in Assay Buffer (20 μl) was then added and the plate was incubated for 10 min at room temperature with gentle shaking.

A Coupling Enzyme Mix containing acyl-coenzyme A oxidase (Sekisui Diagnostics, catalog number T-17), acyl-Coenzyme A synthetase (Sekisui Diagnostics, catalog number T-16), and horseradish peroxidase (Sekisui Diagnostics), ATP (Sigma, catalog number A7699) and coenzyme A (MP Biomedicals, catalog number 100493) in Assay Buffer was prepared. Catalase agarose beads (Sigma, catalog number C9284) were added to the Coupling Enzyme Mix, and the mixture was incubated at 4° C. for 30 min with shaking, and the catalase agarose beads were then removed by centrifugation.

Human VLDL (Millipore, catalog number LP1) was diluted in Assay Buffer, treated with catalase agarose beads for 30 min, and the beads were removed from the solution by centrifugation. Amplex Red (Invitrogen, catalog number A12222) in Assay Buffer was added to a concentration of 33 μM.

To the solution in the sample plate containing LPL, ANGPTL4 and ANGPTL4 antibody, Coupling Enzyme Mix (20 μl) was added, and 54 μl of the resulting solution was transferred to the assay plate. To initiate the lipoprotein lipase reaction, VLDL/Amplex Red solution (18 μl) was added, and resorufin fluorescence was monitored continuously for 30 minutes using an EnVision multiwell plate reader (Perkin Elmer). Final assay concentrations were: 9.4 nM ANGPTL4, ˜4 nM human lipoprotein lipase, 2.3 μg/ml human VLDL, 0.75 mM ATP, 90 μM coenzyme A, 0.5 U/ml ACO, 1.25 U/ml ACS, 1.2 U/ml HRP, and 10 UM Amplex Red.

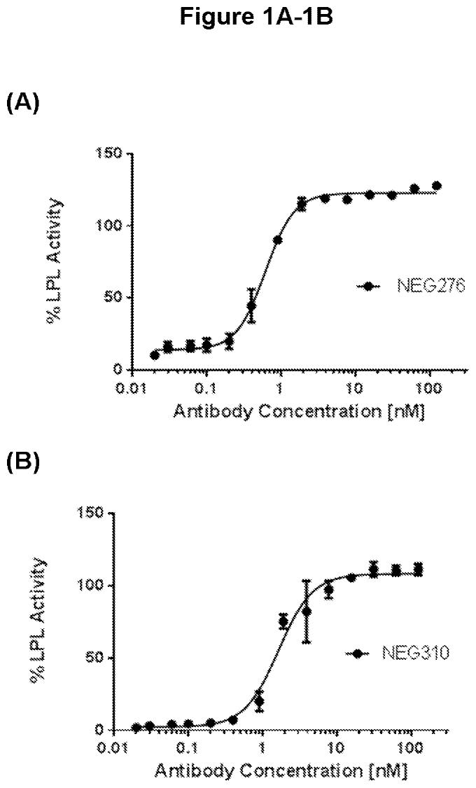

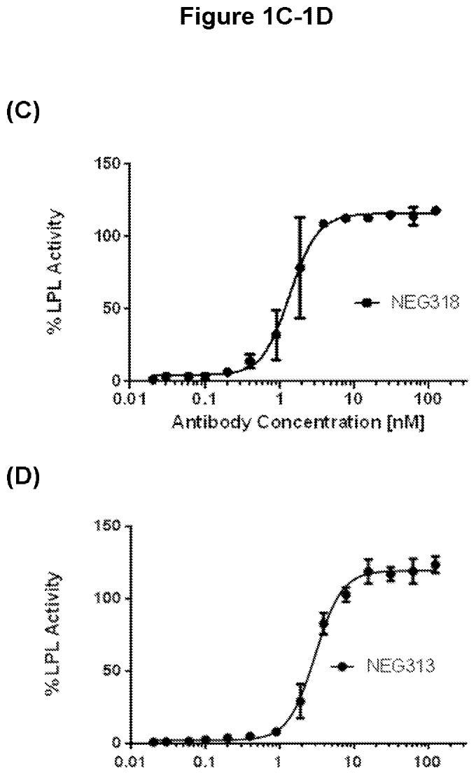

The resulting resorufin fluorescence vs. time data was used to determine lipoprotein lipase enzyme activity (initial rate) for each sample. Control samples without LPL (background control) or without ANGPTL4 or ANGPTL4 antibodies (LPL activity control) were used to normalize the enzyme activity, which was expressed as a percentage of the LPL control activity. Enzyme activity data for different ANGPTL4 antibody concentrations was plotted using GraphPad Prism software, and the data was fitted to generate an EC₅₀ value for the ANGPTL4 antibody-mediated increase in lipoprotein lipase enzyme activity. In this assay, human ANGPTL4 at 10 nM concentration typically inhibited LPL activity by 70-95%. ANGPTL4 antibodies of the invention dose-dependently reversed LPL inhibition by ANGPTL4. EC50 results from this assay are shown in Table 4. Representative data for selected antibodies of the invention is shown in FIG. 1.

TABLE 4
ANGPTL4 antibodies of the invention block human ANGPTL4
inhibition of lipoprotein lipase (LPL).

| | Human ANGPTL4 |
|---|---|
| Antibody | EC₅₀ (nM) |
| NEG276 | 0.6 |
| NEG276-LALA | 0.7 |
| NEG278 | 0.7 |
| NEG310 | 1.6 |
| NEG313 | 3 |
| NEG315 | 3 |
| NEG318 | 1.4 |
| NEG319 | 0.5 |

### Example 8: Preparation of Cynomolgus Monkey, Mouse and Rat ANGPTL4 and Human ANGPTL3 Proteins for Use in Antibody Characterization

The sequence of cynomolgus monkey ANGPTL4 was determined by amplifying the gene sequence from a cynomolgus monkey liver cDNA library (Biochain, catalog number C1534149-Cy, lot no B409051). The primers 5-UT-Cyno 5′-ATCCCCGCTCCCAGGCTAC-3′ (SEQ ID NO: 158) and 3-UT-cyno 5′-CAGCAAGGAGTGAAG-CTCCATGCC-3′ (SEQ ID NO: 159) were designed based on the 5′ and 3′ untranslated regions of human ANGPTL4 cDNA (NCBI sequence NM_139314.2). The gel purified PCR product was ligated into pCR4-Blunt-TOPO (Life Technologies, catalog number K2875-40) and sequenced. The cloned cynomolgus monkey ANGPTL4 cDNA encoded a 406 amino acid protein with 95% homology to human ANGPTL4. The nucleic acid sequence encoding amino acids 26-406 of cynomolgus monkey ANGPTL4 was subcloned into the mammalian expression vector pRS5, to produce the plasmid pRS-Ikk-cynoANGPTL4 (26-406)-FLAG-6HIS-Avi, which has a 20 amino acid Ikk signal sequence, amino acids 26-406 of cyno ANGPTL4, and carboxyl-terminal FLAG, 6HIS, and Avi tags (SEQ ID NO: 160 in Table 5).

The cynomolgus monkey ANGPTL4 (26-406)-FLAG-6HIS-Avi protein was expressed and purified using similar methods as described for human ANGPTL4 (26-406)-FLAG-6HIS-Avi protein in Example 1. For some applications, the purified cynomolgus monkey ANGPTL4 protein was site-specifically biotinylated using a similar as described for human ANGPTL4 protein in Example 1. The purity of the purified cynomolgus monkey ANGPTL4 protein as assessed by SDS-PAGE was >90%.

Cynomolgus monkey ANGPTL4 (26-161)-FLAG-6HIS-Avi protein was prepared using similar methods. The sequence of the cyno ANGPTL4 (26-161) protein encoded by its corresponding expression construct is shown in Table 5 (SEQ ID NO: 161).

Expression, purification, and biotinylation of mouse ANGPTL4 (amino acids 26-410) and rat ANGPTL4 (amino acids 24-405) was carried out using essentially the same methods as described for human ANGPTL4 in Example 1, Method 2. The sequences of the mouse and rat ANGPTL4 proteins encoded by the corresponding expression constructs is shown in Table 5 (SEQ ID NO: 162 and 163, respectively). The purity of the purified mouse ANGPTL4 and rat ANGPTL4 proteins as assessed by SDS-PAGE was >90%.

ANGPTL3 (SEQ ID NO: 5, Table 1) is a human protein that is closely related to ANGPTL4. To enable evaluation of possible binding of antibodies of the invention to ANGPTL3, a human ANGPTL3 (14-460)-FLAG-His-Avi protein (SEQ ID NO: 164, Table 5) was expressed, purified and biotinylated using similar methods as described for human ANGPTL4 in Example 1, Method 2.

TABLE 5
Amino acid sequences of cynomolgus monkey ANGPTL4(26-406)-FLAG-
His6-Avi, mouse ANGPTL4(26-410)-FLAG-His6-Avi, rat ANGPTL4(24-405)-FLAG-His6-
Avi, and human ANGPTL3(17-460)-FLAG-His-Avi (signal peptides are highlighted by
underlining, and the N-terminal QP sequence after the signal peptide and the C-
terminal FLAG-His6-Avi sequences are highlighted with italics)

| | SEQ ID | |
|---|---|---|
| Construct | NO. | Amino Acid Sequence |
| Cynomolgus monkey | 160 | MKTFILLLWVLLLWVIFLLPGATAQPGPVQSKSPRFASWDEMNVLA |
| ANGPTL4(26-406)- | | HGLLQLGQGLREHAERTRSQLNALERRLSACGSACQGTEGSTALPL |
| FLAG-His6-Avi | | APESRVDPEVLHSLQTQLKAQNSRIQQLFHKVAQQQRHLEKQHLRI |
| | | QRLQSQVGLLDPKHLDHEVAKPARRKRRPEMAQPVDSAHNASRLHR |
| | | LPRDCQELFEDGERQSGLFEIQPQGSPPFLVNCKMTSDGGWTVIQR |
| | | RHDGSVDFNRPWEAYKAGFGDPQGEFWLGLEKVHSITGDRNSRLAV |
| | | QLQDWDGNAESLQFSVHLGGEDTAYSLQLTEPVASQLGATTVPPSG |
| | | LSVPFSTWDQDHDLRRDKNCAKSLSGGWWFGTCSHSNLNGQYFRSI |
| | | PQQRQELKKGIFWKTWRGRYYPLQATTMLIQPTAAEAASDYKDDDD |
| | | KHHHHHHGGGLNDIFEAQKIEWHE |
| Cynomolgus monkey | 161 | MKTFILLLWVLLLWVIFLLPGATAQPGPVQSKSPRFASWDEMNVLA |
| ANGPTL4(26-161)- | | HGLLQLGQGLREHAERTRSQLNALERRLSACGSACQGTEGSTALPL |
| FLAG-His6-Avi | | APESRVDPEVLHSLQTQLKAQNSRIQQLFHKVAQQQRHLEKQHLRI |
| | | QRLQSQVGLLDPKHLDHEVAKPARRKRRPEMAQPVDSAHNASRLHR |
| | | LPRDYKDDDDEHHHHHHGGGLNDIFEAQKIEWHE |
| Mouse ANGPTL4(26- | 162 | MKTFILLLWVLLLWVIFLLPGATAQPRPAQPEPPRFASWDEMNLLA |
| 410)-FLAG-His6-Avi | | HGLLQLGHGLREHVERTRGQLGALERRMAACGNACQGPKGKDAPFK |
| | | DSEDRVPEGQTPETLQSLQTQLKAQNSKIQQLFQKVAQQQRYLSKQ |
| | | NLRIQNLQSQIDLLAPTHLDNGVDKTSRGKRLPKMTQLIGLTPNAT |
| | | HLHRPPRDCQELFQEGERHSGLFQIQPLGSPPFLVNCEMTSDGGWT |
| | | VIQRRLNGSVDFNQSWEAYKDGFGDPQGEFWLGLEKMHSITGNRGS |
| | | QLAVQLQDWDGNAKLLQFPIHLGGEDTAYSLQLTEPTANELGATNV |
| | | SPNGLSLPFSTWDQDHDLRGDLNCAKSLSGGWWFGTCSHSNLNGQY |
| | | FHSIPRQRQERKKGIFWKTWKGRYYPLQATTLLIQPMEATAASDYK |
| | | DDDDEHHHHHHGGGLNDIFEAQKIEWHE |
| Rat ANGPTL4(24- | 163 | MKTFILLLWVLLLWVIELLPGATAQPQGRPAQPEPPRFASWDEMNL |
| 405)-FLAG-His6-Avi | | LAHGLLQLGHGLREHVERTRGQLGALERRMAACGNACQGPKGTDPK |
| | | DRVPEGQAPETLQSLQTQLKAQNSKIQQLFQKVAQQQRYLSKQNLR |
| | | IQNLQSQIDLLTPTHLDNGVDKTSRGKRLPKMAQLIGLTPNATRLH |
| | | RPPRDCQELFQEGERHSGLFQIQPLGSPPFLVNCEMTSDGGWTVIQ |
| | | RRLNGSVDFNQSWEAYKDGFGDPQGEFWLGLEKMHSITGDRGSQLA |
| | | VQLQDWDGNAKLLQFPIHLGGEDTAYSLQLTEPTANELGATNVSPN |
| | | GLSLPFSTWDQDHDLRGDLNCAKSLSGGWWFGTCSHSNLNGQYFHS |
| | | IPRQRQQRKKGIFWKTWKGRYYPLQATTLLIQPMEATAASDYKDDD |
| | | DKHHHHHHGGGLNDIFEAQKIEWHET |
| Human ANGPTL3(17- | 164 | MKTFILLLWVLLLWVIFLLPGATAQPSRIDQDNSSFDSLSPEPKSR |
| 460) | | FAMLDDVKILANGLLQLGHGLKDEVHKTKGQINDIFQKLNIFDQSF |
| | | YDLSLQTSEIKEEEKELRRTTYKLQVKNEEVKNMSLELNSKLESLL |
| | | EEKILLQQKVKYLEEQLTNLIQNQPETPEHPEVTSLKTFVEKQDNS |
| | | IKDLLQTVEDQYKQLNQQHSQIKEIENQLRRTSIQEPTEISLSSKP |
| | | RAPRTTPFLQLNEIRNVKHDGIPAECTTIYNRGEHTSGMYAIRPSN |
| | | SQVFHVYCDVISGSPWTLIQHRIDGSQNFNETWENYKYGFGRLDGE |
| | | FWLGLEKIYSIVKQSNYVLRIELEDWKDNKHYIEYSFYLGNHETNY |
| | | TLHLVAITGNVPNAIPENKDLVESTWDHKAKGHENCPEGYSGGWWW |
| | | HDECGENNLNGKYNKPRAKSKPERRRGLSWKSQNGRLYSIKSTKML |
| | | IHPTDSESFEDYKDDDDKHHHHHHGGGLNDIFEAQKIEWHE |

### Example 9: Characterization of Antibody Binding Specificity by Direct ELISA Assays

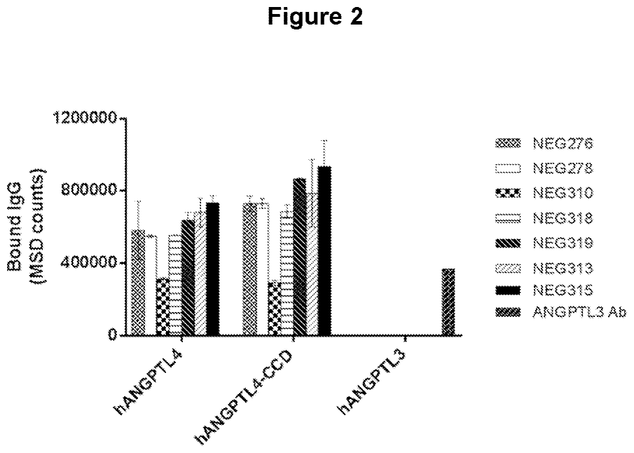

Direct ELISA assays were conducted to characterize antibody binding specificity of selected antibodies of the invention. The assay was performed as follows. A 384-well streptavidin-coated Meso Scale Discovery (MSD) plate was blocked by incubating the plate with 50 μL Blocking Buffer (PBS, pH 7.4, 5% w/v bovine serum albumin) per well at 22° C. for 1 hour with constant shaking (600 rpm). The blocked MSD plate was then washed 3 times with Wash Buffer (PBS, pH 7.4 and 0.05% v/v Tween-20) using a plate washer (BioTek). Following washing, biotinylated human ANGPTL4 proteins diluted in Assay Buffer (PBS, pH 7.4 without CaCl₂) or MgCl₂, 0.5% w/v fatty acid-free bovine serum albumin and 0.02% v/v Tween-20) were immobilized on the surface by incubation at 1 nM concentration (15 μL per well) at 22° C. for one hour: full length human ANGPTL4 (hANGPTL4), human ANGPTL4 coiled-coil domain (hANGPTL4-CCD) and full length human ANGPTL3 (hANGPTL3). The plate was then washed 3 times as described earlier. Antibody diluted to 1 nM concentration in Assay Buffer was then applied to the MSD plate (15 μL per well), and the plate was incubated for 1 hour at 22° C. with constant shaking (600 rpm). Bound antibodies were detected by adding 15 μL per well of a 1:500 dilution of Sulfo-tagged goat anti-human IgG. The plate was then incubated for one hour with constant shaking (600 rpm). The plate was washed 3 times, and then 15 μL/well of 1×MSD read buffer T was added and the plate was developed using a Sector Imager 6000 (Meso Scale Discovery). The data were transferred to Microsoft Excel for analysis and plotted using GraphPad Prism v6. These experiments showed that all of the antibodies of the invention tested in this assay bind to full-length human ANGPTL4 and to the N-terminal domain of human ANGPTL4, and do not bind to full-length human ANGPTL3. An ANGPTL3-specific reference antibody was used as a positive control for the ANGPTL3 binding assay (FIG. 2).

### Example 10: Antibody Dissociation Constants Determination by Solution Equilibrium Titration (SET) Assay

SET assays were performed as follows. In a 96-well polypropylene plate, a constant concentration of ANGPTL4 antibody (10 pM) was mixed with different concentrations of non-biotinylated human, cyno, mouse, or rat full-length ANPGLT4 protein, or human ANGPTL4 N-terminal domain protein (5-fold serial dilution ranging from 0.01 pM to 100 nM) in SET buffer (PBS, pH 7.4 without CaCl₂) or MgCl₂, 0.5% w/v bovine serum albumin (fatty acid free) and 0.02% v/v Tween-20). The final reaction volume was 80 μL. The plate was sealed using an adhesive film and incubated at 22° C. for 14 hours with constant shaking (300 rpm). During the same period of time, a 384-well streptavidin-coated Meso Scale Discovery (MSD) plate was blocked by incubating the plate with 50 μL blocking buffer (PBS, pH 7.4, 5% w/v bovine serum albumin) per well at 4° C. The blocked MSD plate was washed 3 times with wash buffer (PBS, pH 7.4 and 0.05% v/v Tween-20) using a plate washer (BioTek). Biotinylated ANGPTL4 (full-length human, cyno, mouse, or rat ANGPTL4, or human ANGPTL4 N-terminal domain) protein (1 nM, 15 μL per well) was immobilized on the surface of the streptavidin-coated MSD plate by incubation at 22° C. for one hour with constant shaking (600 rpm). The plate was then washed 3 times as described earlier.

The equilibrium binding reactions (15 μL per well) were applied to the MSD plate with immobilized ANGPTL4 and incubated for 20 min at 22° C. The unbound material was removed by washing the plate 3 times with wash buffer, and the captured antibody was detected by adding 15 μL per well of a 1:500 dilution of Sulfo-tagged goat anti-human IgG (Meso Scale Discovery). The plate was then incubated for one hour with constant shaking (600 rpm). The plate was washed 3 times, and then 15 μL/well of 1×MSD read buffer T was added and the plate was developed using a Sector Imager 6000 (Meso Scale Discovery). The data were transferred to Microsoft Excel for analysis and plotted using GraphPad Prism v6. The K_{D} values were determined by fitting the data to the following equation:

$$y = ( {B}_{max} / ( {C}_{A b} / 2 ) ) * ( ( {C}_{A b} / 2 ) - ( ( ( ( ( ( {C}_{A g} + {C}_{A b} ) + {K}_{D} ) / 2 ) - ( ( ( ( ( ( {C}_{A g} + {C}_{A b} ) + {K}_{D} ) \hat{} 2 ) / 4 ) - ( {C}_{Ag} * {C}_{A b} ) ) \hat{} 0.5 ) ) \hat{} 2 ) / ( 2 * {C}_{A b} ) ) ) ,$$

where Bₘₐₓ is the signal when no ANGPTL4 protein is present in solution, C_{Ab} is the constant concentration of ANGPTL4 antibody in solution, C_{Ag} is the concentration of ANGPTL4 in solution, and K_{D} is the equilibrium dissociation constant. Equilibrium dissociation constants determined using this method are shown in Table 6.

TABLE 6
Dissociation constants (K_{D}) for antibodies of the invention binding to
ANGPTL4 proteins determined by Solution Equilibrium Titration
(SET) assays

| | Human | Cyno | Rat | Mouse |
|---|---|---|---|---|
| | ANGPTL4 | ANGPTL4 | ANGPTL4 | ANGPTL4 |
| Antibody | (26-406) | (26-406) | (24-405) | (26-410) |
| (IgG) | K_{D} (pM) | K_{D} (pM) | K_{D} (pM) | K_{D} (pM) |
| NEG276 | 24 | 14 | >500* | >500* |
| NEG276-LALA | 8 | 7 | >500* | >500* |
| NEG278 | 15 | 20 | >500* | >500* |
| NEG310 | 21 | 22 | >500* | >500* |
| NEG313 | 9 | 15 | >500* | >500* |
| NEG315 | 12 | 21 | >500* | >500* |
| NEG318 | 9 | 17 | >500* | >500* |
| NEG319 | 16 | 8 | >500* | >500* |
| Ref Ab* | 17 | 4 | 517 | 194 |

*No binding signal was detected with the experimental conditions used, indicating a K_{D} value >500 pM. A K_{D} value of 517 pM was determined for binding of an ANGPTL4 reference antibody to rat ANGPTL4.

### Example 11: Antibody Binding Kinetics and Dissociation Constants Determined by Octet Kinetic Binding Assay

Dissociation constants (K_{D}) were determined for selected antibodies of the invention by using an Octet (ForteBio) kinetic binding assay. ForteBio 10× Kinetics Buffer (ForteBio, catalog number 18-5032) was diluted 10-fold with DPBS (Life Technologies, catalog number 14190-136), and the resulting solution was added (0.2 ml per well) to a 96-well plate (Greiner, catalog number 65520). Streptavidin sensors (ForteBio, catalog number 18-5020) were immersed in the solution and equilibrated for at least 10 min at room temperature. In a second 96-well plate (Greiner, catalog number 65520), sensors were washed in 1× Kinetics buffer, and then immersed in 200 μl of 25 nM biotinylated human ANGPTL4 or a biotinylated reference protein (for background subtraction) for 1000 sec at seconds at room temperature. The sensors were then washed in 1× Kinetics buffer for 120 sec, and immersed in 200 μl of ANGPTL4 antibody diluted in 1× Kinetics buffer at various concentrations (serial 2-fold dilutions; the highest concentrations were 12.5 nM or 25 nM; the lowest concentrations ranged from 0.8 to 3.1 nM; 4-6 different antibody concentrations were used for each K_{D} determination), and antibody association was monitored for 480 seconds. The sensors were then transferred to a well containing 200 μl 1× Kinetics buffer, and antibody dissociation was monitored for 1200 seconds. Background-corrected association and dissociation curves were globally fitted by Octet Software (ForteBio) to generate association (kₐ) and dissociation (k_{d}) rate constants, which in turn were used to calculate equilibrium dissociation constants (K_{D}). The resulting data for selected antibodies of the invention is shown in Table 7 and Table 8.

TABLE 7
Human ANGPTL4(26-406) antibody dissociation constants (K_{D})
determined by ForteBio kinetic binding assay

| | Human ANGPTL4(26-406) | | |
|---|---|---|---|
| Antibody | kₐ (M⁻¹s⁻¹) | k_{d} (s⁻¹) | K_{D} (pM) |
| NEG276 | 3.4 × 10⁵ | 8.0 × 10⁻⁶ | 23 |
| NEG278 | 3.0 × 10⁵ | 6.7 × 10⁻⁷ | 17* |
| NEG310 | 2.2 × 10⁵ | 1.1 × 10⁻⁵ | 25 |
| NEG313 | 2.8 × 10⁵ | 1.1 × 10⁻⁵ | 40 |
| NEG315 | 2.8 × 10⁵ | 7.9 × 10⁻⁶ | 29 |
| NEG318 | 2.7 × 10⁵ | 1.2 × 10⁻⁵ | 45 |
| NEG319 | 2.8 × 10⁵ | 1.0 × 10⁻⁵ | 36 |

*Upper limit reported because off-rate is slower than the limit of detection, which is approximately 5 × 10⁻⁶ s⁻¹.

TABLE 8
NEG276-LALA dissociation constants (K_{D}) determined by ForteBio
kinetic binding assays

| | | | | | Average |
|---|---|---|---|---|---|
| | ANGPTL4 | | | | K_{D} |
| Antibody | Protein | kₐ (M⁻¹s⁻¹) | k_{d} (s⁻¹) | K_{D} (pM) | (pM) |
| NEG276- | Human ANGPTL4 | 4.1 × 10⁵ | 5.9 × 10⁻⁶ | 14 | 13 |
| LALA | (26-406) | 3.6 × 10⁵ | 6.0 × 10⁻⁶ | 17 | |
| | | 4.3 × 10⁵ | 4.9 × 10⁻⁷ | ≤12* | |
| | | 6.8 × 10⁵ | 7.0 × 10⁻⁶ | 10 | |
| | Cyno ANGPTL4 | 3.6 × 10⁵ | 5.4 × 10⁻⁶ | 15 | 15 |
| | (26-406) | 3.7 × 10⁵ | 4.1 × 10⁻⁶ | ≤14* | |
| | Rat ANGPTL4 | 1.6 × 10⁵ | 1.2 × 10⁻³ | 7470 | 6343 |
| | (24-405) | 1.7 × 10⁵ | 1.1 × 10⁻³ | 6030 | |
| | | 5.2 × 10⁴ | 2.8 × 10⁻⁵ | 5530 | |
| | Mouse ANGPTL4 | 3.6 × 10⁵ | 2.9 × 10⁻³ | 8250 | 6200 |
| | (26-410) | 4.3 × 10⁵ | 1.8 × 10⁻³ | 4150 | |
| | Human ANGPTL4 | 2.2 × 10⁵ | 3.1 × 10⁻⁶ | ≤23* | 38 |
| | (26-161) | 1.8 × 10⁵ | 9.2 × 10⁻⁶ | 52 | |
| | Cyno ANGPTL4 | 2.7 × 10⁵ | 6.4 × 10⁻⁶ | 24 | 56 |
| | (26-161) | 1.9 × 10⁵ | 1.7 × 10⁻⁵ | 87 | |
| | Human ANGPTL3 | —* | —* | >6000** | >6000** |
| | (17-460) | —* | —* | >6000** | |

*Upper limit reported because the dissociation rate is slower that the limit of detection, which is approximately 5 × 10⁻⁶ s⁻¹.
**No binding was detected at the highest concentration of antibody tested, 25 nM.

### Example 12: Epitope Mapping by Hydrogen-Deuterium Exchange/Mass Spectrometry

Hydrogen-deuterium exchange (HDx) in combination with mass spectrometry (MS) (Woods, 2001) was used to map the binding site of antibodies NEG276 and NEG318 on the ANGPTL4 N-terminal domain. In HDx, exchangeable amide hydrogens of proteins are replaced by deuterium. This process is sensitive to protein structure/dynamics and solvent accessibility and, therefore, able to report on ligand binding. The goal of these experiments was the identification of the epitopes of NEG276 and NEG318 on ANGPTL4.

Automated HDx/MS experiments were performed using methods similar to those described in the literature (Chalmers, 2006). The experiments were performed on a Waters HDx-MS platform, which includes a LEAP autosampler, nanoACQUITY UPLC System, and Synapt G2 mass spectrometer. The deuterium buffer used to label the protein backbone with deuterium was 50 mM D-Tris-HCl (pH 7.4), 500 mM NaCl, 15% glycerol, and 0.1% n-octyl β-D-maltoside; the overall percentage of deuterium in the solution was 82.5%. For human ANGPTL4 (26-161) deuterium labeling experiments in the absence of ANGPTL4 antibody, 300 pmol of human ANGPLT4 (26-161)(1.3 μl) was diluted using 100 μl of the deuterium buffer in a chilled tube and incubated for 25 minutes on a rotator at 4° C. The labeling reaction was then quenched with 100 μl of chilled quench buffer on ice for three minutes. After three minutes, the quenched solution was injected onto the LC-MS system for automated pepsin digestion and peptide analysis. For human ANGPTL4 (26-161) deuterium labeling experiments in the presence of bound ANGPTL4 antibody, 300 pmol of the ANGPTL4 antibody was first immobilized on Thermo Protein G Plus beads and cross-linked using disuccinimidyl suberate (DSS). To perform the labeling experiments, the antibody beads (containing 300 pmol antibody) were incubated with 300 pmol human ANGPTL4 (26-161) for 30 minutes at 4° C. After 30 minutes the beads were washed with 200 μl of Tris buffer. Then 200 μl of chilled deuterium buffer was added and the complex was incubated for 25 minutes at 4° C. After 25 minutes, the labeling reaction was quenched with 125 μl of chilled quench buffer on ice for 2.5 minutes. After spinning the sample for 30 seconds in a centrifuge, the quenched solution was injected onto the LC-MS system for automated pepsin digestion and peptide analysis.

All measurements were carried out using a minimum of three analytical triplicates. All deuterium exchange experiments were quenched using 0.5 M TCEP and 3 M urea (pH 2.5). After quenching, the exchanged antigen was subjected to on-line pepsin digestion using a Poroszyme Immobilized Pepsin column (2.1×30 mm) at 12° C. followed by trapping on a Waters Vanguard HSS T3 trapping column. Peptides were eluted from the trapping column and separated on a Waters CSH C18 1×100 mm column (maintained at 1° C.) at a flow rate of 40 μl/min using a binary eight minute gradient of 2 to 35% B (mobile phase A was 99.9% water and 0.1% formic acid; mobile phase B was 99.9% acetonitrile and 0.1% formic acid).

In these deuterium exchange experiments, peptides covering 87% of the ANGPTL4 N-terminal domain sequence were detected. The detected peptides, and the reduction in deuterium incorporation for each peptide, are indicated in Table 9. The HD×MS mapping experiment identified three regions of the ANGPTL4 N-terminal domain that were significantly protected by both NEG276 and NEG318: amino acids 26-35 (G₂₆PVQSKSPRF₃₅)(SEQ ID NO: 165), amino acids 42-68 (N₄₂VLAHGLLQLGQGLREHAERTRSQLSA₆₈)(SEQ ID NO: 174) and amino acids 69-95 (L₆₉ERRLSACGSACQTEGSTDLPLAPES₉₅)(SEQ ID NO: 190). The observation that NEG276 and NEG318 protect multiple regions of the ANGPTL4 N-terminal domain from deuterium incorporation is consistent with results from linear peptide epitope mapping which suggest that NEG276 and NEG318 have conformational rather than linear epitopes (see Example 13).

TABLE 9
Effect of NEG276 and NEG318 binding on deuterium incorporation into
human ANGPLT4(26-161). For each peptide detected by mass spectrometry, the
reduction in deuterium incorporation (in Daltons) for the antibody/ANGPTL4
complex relative to ANGPTL4 alone is shown.

| | | | Reduction in | |
|---|---|---|---|---|
| | | | deuterium | |
| | | | incorporation | |
| Peptide | | | (Daltons) | |
| Name | Sequence | SEQ ID NO | NEG276 | NEG318 |
| 26-35 | GPVQSKSPRF | 165 | 1.1 | 1.2 |
| 28-38 | VQSKSPRFASW | 166 | 1.0 | 0.9 |
| 42-49 | NVLAHGLL | 167 | <0.5 | <0.5 |
| 42-51 | NVLAHGLLQL | 168 | <0.5 | <0.5 |
| 44-51 | LAHGLLQL | 169 | <0.5 | <0.5 |
| 42-54 | NVLAHGLLQLGQG | 170 | 0.8 | 0.8 |
| 42-55 | NVLAHGLLQLGQGL | 171 | 0.9 | 0.8 |
| 42-57 | NVLAHGLLQLGQGLRE | 172 | 0.9 | 0.9 |
| 42-66 | NVLAHGLLQLGQGLREHAERTRSQL | 173 | 1.2 | 1.6 |
| 42-68 | NVLAHGLLQLGQGLREHAERTRSQLSA | 174 | 1.3 | 1.8 |
| 44-66 | LAHGLLQLGQGLREHAERTRSQL | 175 | 1.1 | 1.6 |
| 45-57 | AHGLLQLGQGLRE | 176 | 0.7 | 0.9 |
| 45-66 | AHGLLQLGQGLREHAERTRSQL | 177 | 1.0 | 1.5 |
| 49-66 | LQLGQGLREHAERTRSQL | 178 | 0.5 | 1.0 |
| 52-66 | GQGLREHAERTRSQL | 179 | <0.5 | 0.9 |
| 69-102 | LERRLSACGSACQTEGSTDLPAPESRV | 180 | 1.4 | 1.9 |
| | DPEVL | | | |
| 76-102 | CGSACQTEGSTDLPAPESRVDPEVL | 181 | 1.0 | 1.2 |
| 86-102 | STDLPAPESRVDPEVL | 182 | 1.1 | 1.1 |
| 96-102 | RVDPEVL | 183 | <0.5 | <0.5 |
| 103-109 | HSLQTQL | 184 | 0.5 | 0.5 |
| 110-119 | KAQNSRIQQL | 185 | <0.5 | <0.5 |
| 110-135 | KAQNSRIQQLFHKVAQQQRHLEKQHL | 186 | <0.5 | <0.5 |
| 120-135 | FHKVAQQQRHLEKQHL | 187 | <0.5 | <0.5 |
| 141-147 | QSQFGLL | 188 | 0.6 | 0.6 |
| 144-155 | FGLLDHKHLDHE | 189 | <0.5 | <0.5 |

### Example 13: Epitope Mapping by Linear Peptide Binding

The ability of selected antibodies of invention, namely NEG276 and NEG318, to bind to linear 15-amino-acid peptides derived from the N-terminal coiled coil domain of human ANGPTL4 was tested. A total of 43 peptides were synthesized and purified using standard methods; the sequence of the peptides are shown in Table 10. The peptides were immobilized on a glass surface, and the ability of NEG276 and NEG318 to bind to the immobilized peptides was evaluated using experimental methods optimized for linear peptide epitope mapping at JPT Peptide Technologies (Berlin, Germany). An antibody that does not bind to ANGPTL4 was used as a control for non-specific binding. No specific binding of NEG276 or NEG318 to any of the 43 15-mer peptides was observed in these experiments. These results strongly suggest that the epitopes of NEG276 and NEG318 are not linear ANGPTL4 peptides, but instead are conformational epitopes.

TABLE 10
Sequences of linear ANGPTL4-derived peptides
used for peptide binding experiments.

| | Name |
|---|---|
| Peptide | (SEQ ID NO.) |
| GPVQSKSPRFASWDE | P1 (191) |
| QSKSPRFASWDEMNV | P2 (192) |
| SPRFASWDEMNVLAH | P3 (193) |
| FASWDEMNVLAHGLL | P4 (194) |
| WDEMNVLAHGLLQLG | P5 (195) |
| MNVLAHGLLQLGQGL | P6 (196) |
| LAHGLLQLGQGLREH | P7 (197) |
| GLLQLGQGLREHAER | P8 (198) |
| QLGQGLREHAERTRS | P9 (199) |
| QGLREHAERTRSQLS | P10 (200) |
| REHAERTRSQLSALE | P11 (201) |
| AERTRSQLSALERRL | P12 (202) |
| TRSQLSALERRLSAS | P13 (203) |
| QLSALERRLSASGSA | P14 (204) |
| ALERRLSASGSASQG | P15 (205) |
| RRLSASGSASQGTEG | P16 (206) |
| SASGSASQGTEGSTD | P17 (207) |
| GSASQGTEGSTDLPL | P18 (208) |
| SQGTEGSTDLPLAPE | P19 (209) |
| TEGSTDLPLAPESRV | P20 (210) |
| STDLPLAPESRVDPE | P21 (211) |
| LPLAPESRVDPEVLH | P22 (212) |
| APESRVDPEVLHSLQ | P23 (213) |
| SRVDPEVLHSLQTQL | P24 (214) |
| DPEVLHSLQTQLKAQ | P25 (215) |
| VLHSLQTQLKAQNSR | P26 (216) |
| SLQTQLKAQNSRIQQ | P27 (217) |
| TQLKAQNSRIQQLFH | P28 (218) |
| KAQNSRIQQLFHKVA | P29 (219) |
| NSRIQQLFHKVAQQQ | P30 (220) |
| IQQLFHKVAQQQRHL | P31 (221) |
| LFHKVAQQQRHLEKQ | P32 (222) |
| KVAQQQRHLEKQHLR | P33 (223) |
| QQQRHLEKQHLRIQH | P34 (224) |
| RHLEKQHLRIQHLQS | P35 (225) |
| EKQHLRIQHLQSQFG | P36 (226) |
| HLRIQHLQSQFGLLD | P37 (227) |
| IQHLQSQFGLLDHKH | P38 (228) |
| LQSQFGLLDHKHLDH | P39 (229) |
| QFGLLDHKHLDHEVA | P40 (230) |
| LLDHKHLDHEVAKPA | P41 (231) |
| HKHLDHEVAKPARRK | P42 (232) |
| KHLDHEVAKPARRKR | P43 (233) |

### Example 14: Effect of ANGPTL4 Antibodies of the Invention on Plasma Triglyceride Concentrations in Human ANGPTL4 Transgenic Mice

A construct to express transgenic human ANGPTL4 in mice was made by inserting the full-length human ANGPTL4 cDNA sequence into the polylinker region of the pLIVLE6 vector, which contains the human apolipoprotein E gene promoter and its hepatic control region. ANGPTL4 transgenic mice were generated on a C57BL/6 J background and bred at Novartis (East Hanover, NJ). Transgenic mice were tail-clipped at 7 days of age and DNA was extracted from the tails using a REDExtract-N-Amp Tissue PCR Kit (Sigma-Aldrich; St. Louis, MO; cat #XNATR). The human ANGPTL4 transgene was detected by using primer pairs targeting the pLIVLE6 vector and targeting ANGPTL4 cDNA. Mice were housed in solid-bottom cages on a rack equipped to automatically provide water ad libitum, maintained on a 12 hr light/dark cycle (6 am to 6 pm), and given standard rodent chow (Harlan-Teklad; Frederick, MD; cat #8604). The vivarium was maintained between 68-76° F. with 30-70% humidity. Mice were housed with littermates and received food and water ad libitum during the study, except for 4 hr fasts prior to sample collection.

Animals were fasted for 4 hr and briefly anesthetized for submandibular blood collection to measure baseline plasma triglyceride concentrations. Mice were then injected intraperitoneally (i.p.) with 30 mg/kg antibody diluted in PBS (10 mL/kg injection volume). Blood was collected after 4 hr fasts on days 1, 2, and 5 post-dose to measure plasma triglyceride and total human IgG concentrations. Blood was collected into BD Microtainer collection/separator tubes with EDTA (Becton, Dickinson, and Company; Franklin Lakes, NJ, catalog number 365973). Samples were centrifuged for 10 min at 20,817×g, and plasma was transferred to a 0.2 mL Thermo-strip tube (Thermo-Scientific; Pittsburgh, PA; cat #AB 0451) and frozen and stored at −80° C.

Plasma triglyceride concentrations were measured using the Triglyceride (GPO) Liquid Reagent set (Pointe Scientific, Canton, MI, catalog number T7532-500). Briefly, 300 μL of assay reagent, pre-warmed to 37° C., was added to 5 μL of plasma in a clear, flat-bottom 96-well plate (Thermo Scientific, catalog number 269620). The plate was mixed on a plate shaker for 30 sec and then placed in a 37° C. incubator for 5 min. Following a 20 sec mix, absorbance at 500 nm was measured using a Molecular Devices SPECTRAmax PLUS plate reader. Triglyceride concentrations were calculated by using a calibration curve generated using known quantities of a triglyceride standard (Pointe Scientific, catalog number T7531-STD).

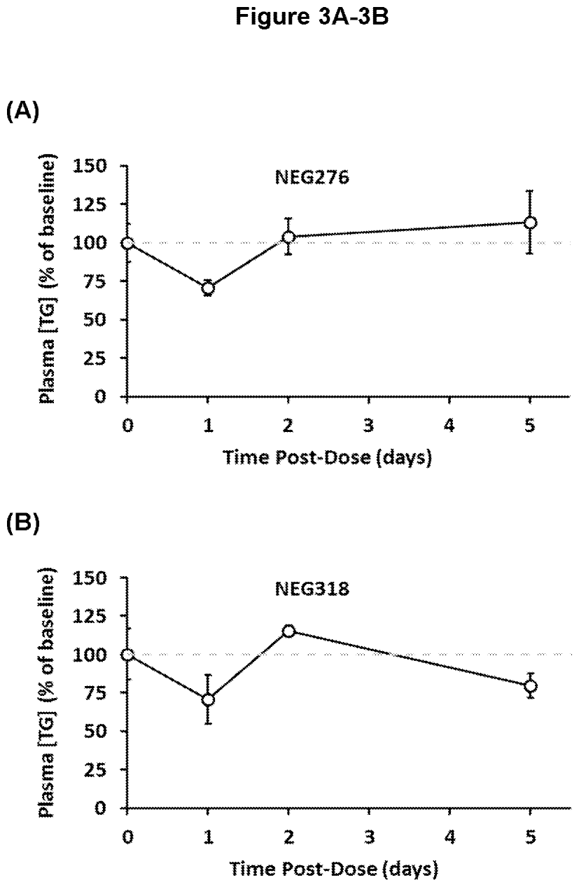

The antibodies NEG276 and NEG318 both reduced plasma triglyceride levels when administered to the human ANGPTL4 transgenic mice (FIG. 3).

### Example 15: Effects of Administering One of the ANGPTL4 Antibodies of the Invention to Obese, Diabetic, Hypertriglyceridemic Cynomolgus Monkeys

To evaluate the pharmacokinetic profile and pharmacological effects of NEG276-LALA, we administered a single, subcutaneous, 3 mg/kg dose to four hypertriglyceridemic cynomolgus monkeys. The monkeys used in this study had baseline plasma triglyceride levels ranging from 207 mg/dl to 2438 mg/dL. At various timepoints over 5 weeks after NEG276-LALA dosing, plasma samples were collected (blood samples were drawn from animals prior to morning feeding, but the animals were not fasted overnight).

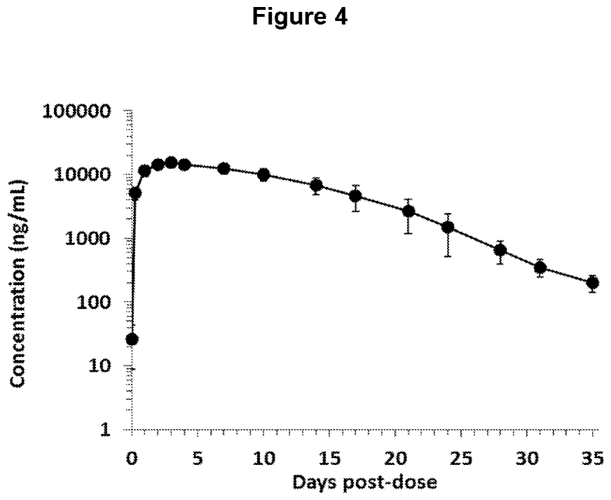

Total NEG276-LALA plasma concentrations were determined by standard methods. NEG276-LALA reached an average maximum plasma concentration (Cₘₐₓ) of 15,536±2281 ng/ml at 3 days post-dose. At day 21 post-dose, the average NEG276-LALA plasma concentration was 2663 ng/ml (FIG. 4).

Plasma TG, total cholesterol, high-density lipoprotein (HDL) cholesterol, total apolipoprotein B (apoB), and apolipoprotein CIII (apoCIII) concentrations were determined using commercially available assay kits (TG: Triglyceride (GPO) Liquid Reagent set, Pointe Scientific, catalog number T7532-500; total cholesterol: Cholesterol Reagent Set, Pointe Scientific, catalog number C7510-500; HDL: Cholesterol Precipitating Reagent from manual HDL reagent kit, Wako, catalog number 431-52501; total ApoB: K-Assay Apo B, Kamiya Biomedical Company, catalog number KAI-004; ApoC-III: ApoC-III Assay Reagent, Randox, catalog number LP-3865).

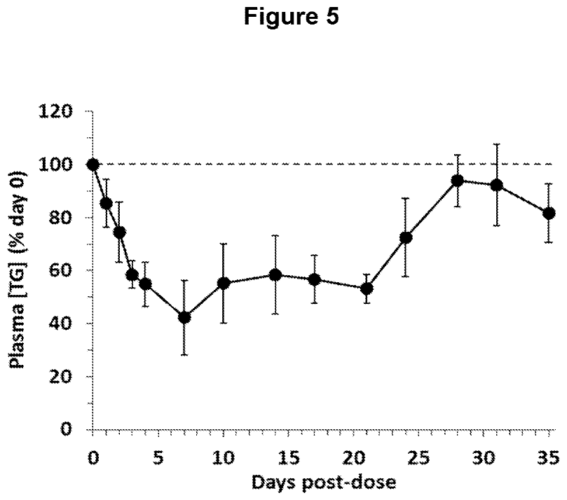

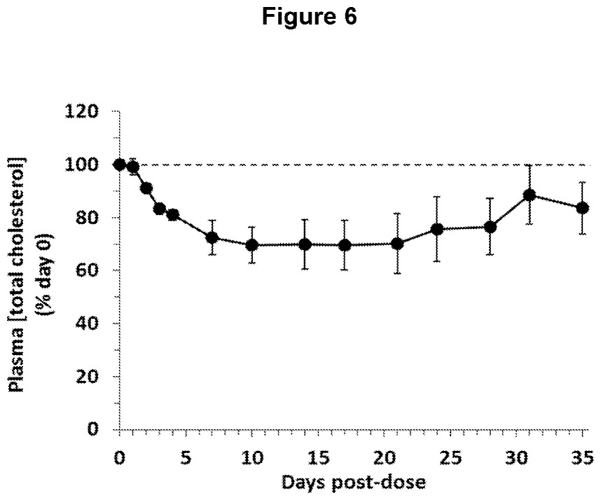

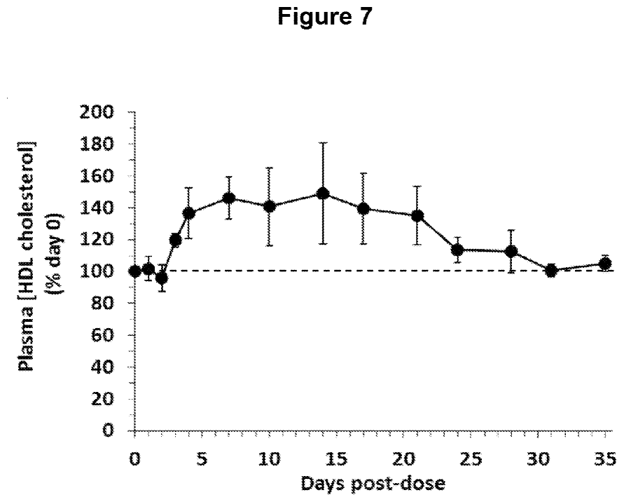

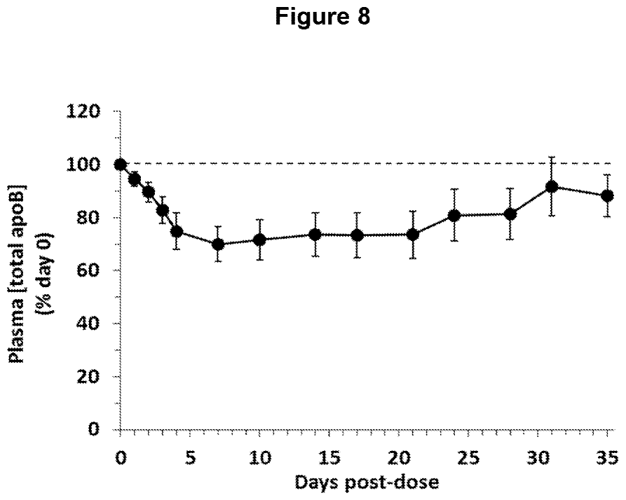

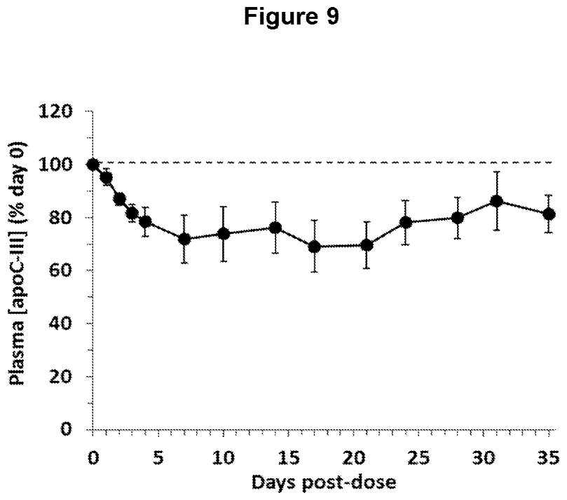

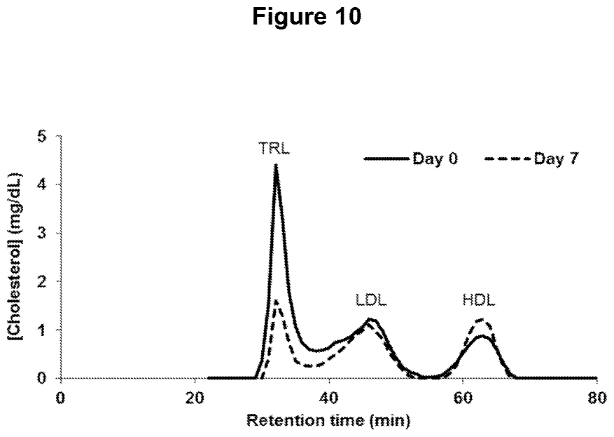

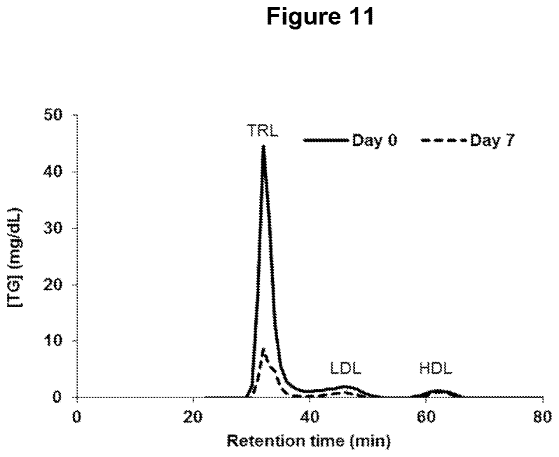

NEG276-LALA administration resulted in a marked decrease in plasma triglyceride (TG) levels. Peak plasma TG lowering was observed on day 7 post-dosing; at this time point plasma TG concentrations were 58% lower than baseline plasma TG levels. After peak TG lowering occurred on day 7 post-dose, plasma TG concentrations remained suppressed by greater than 40% relative to baseline concentrations through day 21 post-dose, then returned to baseline (FIG. 5). In addition to its effect on plasma TG, NEG276-LALA administration reduced plasma total cholesterol concentrations by approximately 30% relative to baseline (FIG. 6) and increased HDL cholesterol concentrations by more than 20% from baseline on days 7 through 21 post-dosing (FIG. 7). In addition, an approximately 30% decrease in plasma total apoB concentrations was observed on days 7 through 21 post-dose (FIG. 8), and an approximately 25% decrease in plasma apoC-III concentrations relative to baseline was observed on days 7 through 21 (FIG. 9). We also evaluated the effect of NEG276-LALA administration on lipoprotein-associated triglyceride and cholesterol levels by separating lipoprotein components using standard size-exclusion chromatography methods. Comparison of lipoprotein profiling data for pre-dose (day 0) and day 7 post-dose samples showed that NEG276-LALA administration resulted in marked decreases in triglyceride-rich lipoprotein (TRL) associated cholesterol and triglyceride concentrations (results from one monkey are shown in FIG. 10 and FIG. 11).

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1.-33. (canceled)

34. A polynucleotide encoding an anti-ANGPTL4 antibody or antigen-binding fragment thereof comprising:
(a) a heavy chain variable region comprising a HCDR1 of SEQ ID NO: 7, a HCDR2 of SEQ ID NO: 8, and a HCDR3 of SEQ ID NO: 9, and a light chain variable region comprising a LCDR1 of SEQ ID NO: 17, a LCDR2 of SEQ ID NO: 18, and a LCDR3 of SEQ ID NO: 19; or
(b) a heavy chain variable region comprising a HCDR1 of SEQ ID NO: 10, a HCDR2 of SEQ ID NO: 11, and a HCDR3 of SEQ ID NO: 12, and a light chain variable region comprising a LCDR1 of SEQ ID NO: 20, a LCDR2 of SEQ ID NO: 21, and a LCDR3 of SEQ ID NO: 22.

35. The polynucleotide of claim 34, wherein the antibody or antigen-binding fragment thereof is a monoclonal antibody, humanized antibody, single chain antibody, Fab fragment, Fv fragment, F(ab′)₂ fragment, or scFv fragment.

36. The polynucleotide of claim 34, wherein the antibody or antigen-binding fragment thereof is an IgG1 or IgG4 isotype.

37. The polynucleotide of claim 34, wherein the antibody or antigen-binding fragment thereof is a bispecific antibody or antigen-binding fragment thereof.

38. The polynucleotide of claim 34, wherein the antibody or antigen-binding fragment thereof comprises:
(i) a light chain variable region comprising an amino acid sequence that is at least 90% identical to SEQ ID NO: 23, and
(ii) a heavy chain variable region comprising an amino acid sequence that is at least 90% identical to SEQ ID NO: 13.

39. The polynucleotide of claim 34, wherein the antibody comprises:
(i) a heavy chain comprising an amino acid sequence that is at least 90% identical to SEQ ID NO: 15, and
(ii) a light chain comprising an amino acid sequence that is at least 90% identical to SEQ ID NO: 25.

40. The polynucleotide of claim 34, wherein the antibody comprises a heavy chain comprising an amino acid sequence of SEQ ID NO: 15, and a light chain comprising an amino acid sequence of SEQ ID NO: 25.

41. The polynucleotide of claim 34, wherein the antibody comprises:
(i) a heavy chain comprising an amino acid sequence that is at least 90% identical to SEQ ID NO: 28, and
(ii) a light chain comprising an amino acid sequence that is at least 90% identical to SEQ ID NO: 25.

42. The polynucleotide of claim 34, wherein the antibody comprises a heavy chain comprising an amino acid sequence of SEQ ID NO: 28, and a light chain comprising an amino acid sequence of SEQ ID NO: 25.

43. The polynucleotide of claim 34, wherein the polynucleotide comprises:
(i) a nucleic acid sequence having at least 95% sequence identity to SEQ ID NO: 24, and
(ii) a nucleic acid sequence having at least 95% sequence identity to SEQ ID NO: 14.

44. The polynucleotide of claim 34, wherein the polynucleotide comprises:
(i) a nucleic acid sequence having at least 95% sequence identity to SEQ ID NO: 27, and
(ii) a nucleic acid sequence having at least 95% sequence identity to SEQ ID NO: 30.

45. The polynucleotide of claim 34, wherein the polynucleotide comprises:
(i) a nucleic acid sequence having at least 95% sequence identity to SEQ ID NO: 16, and
(ii) a nucleic acid sequence having at least 95% sequence identity to SEQ ID NO: 26.

46. The polynucleotide of claim 34, wherein the polynucleotide comprises:
(i) a nucleic acid sequence having at least 95% sequence identity to SEQ ID NO: 29, and
(ii) a nucleic acid sequence having at least 95% sequence identity to SEQ ID NO: 31.

47. A host cell comprising the polynucleotide of claim 34.

48. An expression vector comprising the polynucleotide of claim 34.

49. The expression vector of claim 48, wherein the expression vector is a viral-based vector.

50. The expression vector of claim 48, wherein the expression vector is a nonviral expression vector.

51. The expression vector of claim 48, wherein the expression vector further comprises a nucleic acid sequence encoding a secretory signal.

52. A method of producing an anti-ANGPTL4 antibody or antigen-binding fragment thereof, comprising culturing a host cell comprising the polynucleotide of claim 34 under conditions suitable for production of the anti-ANGPTL4 antibody or antigen-binding fragment thereof.
